(19) United States
(12) Patent Application Publication
(10) Pub. No.: US 2023/0054569 A1
(43) Pub. Date: Feb. 23, 2023

(54) COMPOSITIONS AND METHODS FOR TREATING RETINITIS PIGMENTOSA

(71) Applicant: Alia Therapeutics Srl, Milano (IT)

(72) Inventors: Antonio Casini, Trento (IT); Laura Pezzè, Trento (IT); Kalina Badowska, Jasieniec (PL)

(73) Assignee: Alia Therapeutics Srl, Milano (IT)

(21) Appl. No.: 17/786,849

(22) PCT Filed: Dec. 17, 2020
(86) PCT No.: PCT/EP2020/086702

§ 371 Date: Jun. 17, 2022

Related U.S. Application Data
(60) Provisional application No. 62/949,888, filed on Dec. 18, 2019.

(51) Int. Cl. A61K 31/7088; C12N 15/10; C12N 15/864

(52) U.S. Cl. CPC A61K 31/7088; C12N 15/102; C12N 15/8645; C12N 2310/20

(57) ABSTRACT

Compositions and methods useful for altering a P347 mutation in a RHO gene.

### 1. CROSS-REFERENCE TO RELATED APPLICATIONS

This application claims the priority benefit of U.S. provisional application No. 62/949,888, filed Dec. 19, 2019, the contents of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Dec. 9, 2020, is named ALA-003WO_SL.txt and is 106,933 bytes in size

### 3. BACKGROUND

Retinitis pigmentosa (RP) refers to a group of inherited diseases causing retinal degeneration. More than 60 genes have been identified that are associated with RP, some of which are autosomal recessive, autosomal dominant, or X-linked. In many cases, RP is caused by mutations in the RHO gene, which encodes the rhodopsin protein. Rhodopsin is an essential photopigment expressed in retinal rod photoreceptor cells that is responsible for the conversion of light stimuli into electrical signals in the first step of phototransduction. Rhodopsin is expressed as a light-sensitive G-protein-coupled receptor that consists of an opsin protein moiety bound to a retinal chromophore, and represents the main component of the disk membranes of rod photoreceptor cell outer segments. Misfolded rhodopsin can contribute to rod photoreceptor cell degeneration and death, and can ultimately lead to blindness.

RP caused by mutations in the RHO gene is typically caused by heterozygous, and rarely by homozygous, mutation in the RHO gene on chromosome 3q22. There are over one hundred naturally occurring mutations in the RHO gene that lead to autosomal dominant RP, some of which are found in the carboxyl-terminus of the rhodopsin protein, for example P347L, P347S, P347R, P347Q, P347T, and P347A mutations. See, Dryja, et al., 1990, N. Engl. J. Med. 323:1302-1307; Concepcion et al., 2002, Vision Research, 42(4):417-426. The use of CRISPR genome-editing for treating autosomal dominant RP caused by mutations at positions such as P23 and R135 has been suggested. See, WO 2019/102381 and WO 2018/009562. WO 2018/009562 notes that R135 mutations are the second most common rhodopsin mutations worldwide after P347 mutations and suggests that R135 mutations are amenable to correction through non-homologous end joining (NHEJ), as introduced premature stop codons will likely result in degraded transcripts through non-sense mediated decay, thereby relieving the dominant negative effect of the R135 mutations. On the other hand, WO 2018/009562 indicates that the position of P347 mutations (in exon 5 toward the carboxyl-terminus of rhodopsin) presents additional challenges for correction by CRISPR genome-editing because, according to WO 2018/009562, premature stope codons in the last exon of a gene are not susceptible to non-sense mediated decay.

Thus, there remains an unmet medical need for compositions and methods for treating RP, and in particular RP caused by P347 mutations in the RHO gene.

### 4. SUMMARY

The disclosure herein addresses needs in the art by providing compositions and methods useful for altering a P347 mutation in a RHO gene. Such compositions and methods are particularly needed, as mutations at codon 347 of the human RHO gene can result in severe diseases including Retinitis Pigmentosa (RP), which can lead to blindness. The disclosed compositions and methods are useful, for example, for cellular manipulations and subject treatments, particularly of RP subjects.

The inventors have discovered that mutant allele specific editing of a human RHO gene having a P347 mutation can be achieved using guide RNA (gRNA) molecules targeting the P347 mutation, and that such allele specific gene editing can promote mutant rhodopsin mRNA destabilization and degradation. Without being bound by theory, it is believed elimination of mutant RHO may arrest photoreceptor death, thus blocking the disease phenotype when the allele-specific targeting strategy is deployed in the retina of a subject with autosomal dominant RP. The inventors have further discovered downregulation of rhodopsin with a P347 mutation, particularly a P347L mutation, can be achieved by a single cut at the level of the P347 mutation, e.g., by using a single gRNA. Yet still further, the inventors have discovered that that rhodopsin having a P347 mutation can also be downregulated by a dual targeting strategy, whereby an allele specific gRNA is used in combination with a gRNA targeting intron 4, thereby promoting deletion of part of RHO exon 5. The inventors have further discovered that a specific gRNA for editing RHO P347L, gRNA Guide 1 described herein having a spacer sequence of SEQ ID NO:5 is particularly good at generating mutant allele specific indels.

In one aspect, the disclosure provides guide RNA (gRNA) molecules, for example Cas9 gRNA molecules, useful, for example, for editing a human RHO gene having a P347 mutation, e.g., a P347L mutation or other P347 mutation such as P347S, P347R, P347Q, P347T, or P347A. gRNAs of the disclosure can be used with a Cas protein, e.g., a Cas9 protein, such as SpCas9 or Nme2Cas9, to cleave a RHO gene having a P347 mutation. The Cas protein can be a wild-type Cas protein or, alternatively, can be a Cas protein variant having one or more mutations relative to a wild-type Cas protein. The inventors have discovered that certain Cas9 variants used in combination with gRNAs of the disclosure are particularly effective at preferentially cleaving a human RHO gene having a P347 mutation over a human RHO gene not having a P347 mutation. Exemplary features of the gRNAs of the disclosure are described in Section 6.2 and numbered embodiments 1 to 144, infra. Exemplary features of Cas proteins (e.g., Cas9 proteins) and Cas9 protein variants that can be used in combination with the gRNAs of the disclosure are described in Section 6.3 and numbered embodiments 162 to 187, infra.

The disclosure further provides nucleic acids encoding gRNAs of the disclosure, nucleic acids encoding Cas9 proteins, pluralities of nucleic acids and host cells comprising the nucleic acids (including pluralities of nucleic acids) of the disclosure. Exemplary features of the nucleic acids and host cells are described in Section 6.4 and numbered embodiments 145 to 199 and 228 to 244, infra.

The disclosure further provides systems, particles, and pluralities of particles containing gRNAs and nucleic acids of the disclosure. Exemplary systems, particles, and pluralities of particles are described in Section 6.5 and numbered embodiments 200 to 226, infra.

The disclosure further provides pharmaceutical compositions comprising the gRNAs, nucleic acids (including pluralities of nucleic acids), particles (including pluralities of particles), and systems the disclosure. Exemplary pharmaceutical compositions are described in Section 6.6 and numbered embodiment 227, infra.

The disclosure further provides cells (e.g., from a subject having a RHO gene with a P347 mutation) and populations of cells comprising the gRNAs, nucleic acids (including pluralities of nucleic acids), particles (including pluralities of particles), and systems of the disclosure. Exemplary cells are described in Section 6.5 and numbered embodiments 228 to 244, infra.

The disclosure further provides methods of using the gRNAs, nucleic acids (including pluralities of nucleic acids), systems, and particles (including pluralities of particles) of the disclosure for altering cells, for example a human cell having a RHO gene having a P347 mutation, e.g., a P347L mutation. Methods of the disclosure can be used, for example, to treat subjects having a RP caused by a P347 mutation in their RHO gene, for example a P347L mutation. Exemplary methods of altering cells are described in Section 6.7 and numbered embodiments 245 to 311, infra.

### 5. BRIEF DESCRIPTION OF THE FIGURES

FIG. 1A-1B: guide RNAs for targeting the RHO P347 locus. Schematic representation of the RHO P347 locus genomic DNA sequence annotated with the positions of guide RNAs for SpCas9 (FIG. 1A) and Nme2Cas9 (FIG. 1B) targeting the P347L mutation (indicated in bold). The arrow at the end of each guide indicates the localization of the downstream PAM on their respective strands. FIGS. 1A and 1B both disclose SEQ ID NO:302.

FIGS. 2A-2D: evaluation of gene editing on the RHO P347 locus. FIG. 2A Indel formation at the RHO P347 wt endogenous locus 3 days after transient transfection of HEK293T/17 cells with wt SpCas9 and three different sgRNAs, as indicated. FIG. 2B Generation of a stable cell pool expressing the RHO P347L mutant. Representative western blot on lysates of HEK293T/17 cells stably transduced with a lentiviral vector expressing RHO P347L (293T-RHO-P347L cells). GAPDH detection was used as a loading control. Untransduced HEK293T/17 cells were blotted as a negative control to demonstrate antibody specificity. FIG. 2C Indel formation at the integrated RHO P347L locus 3 days after transient transfection of 293T-RHO-P347L cells with wt SpCas9 together with three different guide RNAs, as indicated. FIG. 2D Evaluation of indel formation at the integrated RHO P347L locus in HEK293T/17 (293T-RHO-P347L) and RPE1 (RPE-RHO-P347L) cell pools stably expressing the RHO mutant 9 days after transduction with a lentiviral vector expressing wt SpCas9 and the Guide 1 sgRNA followed by puromycin selection. In FIG. 2A and FIG. 2C data are presented as mean±SEM for n=3 biologically independent studies.

FIG. 3: evaluation of gene editing at the integrated RHO P347L locus using a panel of high-fidelity SpCas9 variants (SpCas9 E containing the mutation K526E; ES containing the mutation K526E+R661S; ESN containing the mutation K526E+R661S+Y515N; evoCas9 containing the mutations M495V, Y515N, K526E, and R661Q). Indel formation measured 3 days after transient transfection of 293T-RHO-P347L cells with wt SpCas9 or high-fidelity mutants together with sgRNAs Guide 1 or Guide 2, as indicated. Data are presented as mean±SEM for n=3 biologically independent studies.

FIGS. 4A-4E: Allele-specific knock-out of the RHO P347L mutation. FIG. 4A Indel formation at the integrated RHO P347L locus (on-target, black bars) and at the wt endogenous RHO P347 locus (off-target, grey bars) measured at 3 days after transient transfection of wt SpCas9 or high-fidelity SpCas9 variants as described in FIG. 3, as indicated, together with sgRNA Guide 1 using the TransIT-LT1 reagent. The on-/off-target ratio for each variant is reported above the respective bars on the graph. FIG. 4B Indel formation at the integrated RHO P347L locus (on-target, black bars) and at the wt endogenous RHO P347 locus (off-target, grey bars) measured at 3 days after transient transfection of wt SpCas9 or high-fidelity SpCas9 variants, as indicated, together with sgRNA Guide 1 using the Lipofectamine 3000 reagent. The on-/off-target ratio for each variant is reported above the respective bars on the graph FIG. 4C Representative western blot showing RHO P347L intracellular levels in 293T-RHO-P347L cells 6 days after transfection with either wt SpCas9 or one of the SpCas9 high-fidelity variants, as indicated, together with Guide 1 sgRNA or a non-targeting sgRNA (empty). GAPDH detection was used as a loading control. FIG. 4D Representative western blot on lysates of 293T-RHO-wt cells transfected with wt SpCas9 together with the NGG2 sgRNA targeting RHO. Cells transfected with wt SpCas9 and a mock targeting sgRNA are reported as controls. Untreated HEK293T/17 cells were blotted as a negative control to demonstrate antibody specificity. In FIG. 4A data are presented as mean±SEM for n=3 (RHO P347L, black bars) or n=2 (RHO wt, grey bars) biologically independent studies. FIG. 4E: Indel formation at RHO WT locus after transient transfection with wt SpCas9 and a sgRNA (NGG2, Table 5) targeting the RHO coding sequence located at the N-terminus of the protein.

FIG. 5: indel formation after 3 days at the endogenous RHO P347 wt locus in HEK293T/17 cells transiently transfected with Nme2Cas9 and two different sgRNAs, as indicated.

FIGS. 6A-6E: Editing of the RHO P347L allele in reporter cell lines. FIG. 6A: RHO P347L transgene copy number measured in different clones (clone 1 to 7) of 293T-RHO-P347L cells by qPCR. The GAPDH gene, present in 2 copies in the genome of HEK293T cells, has been used to calculate the number of input genomes analyzed in the assay. FIG. 6B and FIG. 6C: Standard curves for the RHO P347L transgene and for GAPDH used to calculate the absolute number of copies of each target for RHO P347L copy number evaluation as reported in FIG. 6A. FIG. 6D: Indel formation at the RHO P347L locus and allele-specificity against the endogenous RHO wt locus were evaluated 3 days after transient transfection of 293T-RHO-P347L clone 4 with wt SpCas9 or the different high-fidelity SpCas9 variants together with Guide 1. FIG. 6E Indel formation at the RHO P347L locus was evaluated 3 days after transient transfection of the 293TetO-RHO-P347L cell pool with wt SpCas9 or the different high-fidelity SpCas9 variants together with Guide 1. In FIG. 6D data are presented as mean±SEM for n=3 biologically independent studies, while in FIG. 6E data are presented as mean±SEM for n=2 biologically independent studies.

FIGS. 7A-7B: Evaluation of the functional consequences of RHO P347L editing. FIG. 7A: RHO P347L mRNA levels were measured using qPCR in 293T-RHO-P347L clone 4 at 7 days post transfection with Guide 1 together with wt SpCas9 or different high-fidelity SpCas9 variants. Fold change is reported over mock-treated sample (SpCas9 empty). FIG. 7B: Evaluation of RHO P347L protein intracellular levels in 293TetO-RHO-P347L cell pool after editing with wt SpCas9 and different high-fidelity SpCas9 variants in combination with Guide 1. Cells were collected at 7 days after transfection for protein extraction. In FIG. 7A data are presented as mean±SEM for n=3 biologically independent studies.

FIGS. 8A-8B. Evaluation of the specificity of the RHO P347L targeting strategy. FIG. 8A: Identification of genome-wide off-targets for Guide 1 in combination with wt SpCas9 (left panel) and the ES, EQ, ESN, EQN high-fidelity SpCas9 variants (right panel) using the GUIDE-seq protocol in 293T-RHO-P347L clone 4. FIG. 8A discloses the sequences in the left panel as SEQ ID NOS 308 and 304-325 and the sequences in the right panel as SEQ ID NOS 303, 303, 303, and 303, respectively, in order of appearance. The reads indicated for each detected site are a measure on how often the site is cleaved. On-target site is indicated with an arrow on the left diagram. Mismatched positions are indicated as shaded boxes. FIG. 8B: Validation of the top 3 off-target sites detected by GUIDE-seq. Indel formation at the 3 most cleaved off-target sites according to GUIDE-seq was measured after transient transfection of 293T-RHO-P347L clone 4 with Guide 1 together with either wt SpCas9 or the ES, EQ, ESN, EQN high-fidelity SpCas9 variants using Sanger sequencing chromatogram deconvolution (see Methods). In FIG. 8B data are presented as mean±SEM for n=3 biologically independent studies.

FIGS. 9A-9B: Design of a targeting strategy for RHO P347L based on genomic deletions. FIG. 9A: Schematic representation of the dual sgRNA approach used to target the RHO P347L mutation. An allele-specific sgRNA targets the RHO P347L mutant locus while a bi-allelic cut in RHO intron 4 allows the generation of a deletion only in the mutated allele. FIG. 9B: Schematics depicting the position of the three sgRNAs targeting RHO intron 4 used to generate the deletion in Example 3. FIG. 9B discloses SEQ ID NO:326.

FIGS. 10A-10D: Validation of sgRNA activity in cultured cells. FIG. 10A: Evaluation of indel formation in RHO intron after transient transfection of 293TetO-RHO-P347L cells with wt SpCas9 together with each intron-targeting sgRNA (indicated as sg-Int39, sg-Int103, sg-Int153). FIG. 10B: Evaluation of deletion formation at the RHO P347L locus after double-sgRNA targeting. 293TetO-RHO-P347L cells were transiently transfected with wt SpCas9 and Guide 1 (targeting the RHO P347L mutation) in combination with each of the three intron-targeting guides. As a control, each guide was tested also singularly. PCR products obtained with primers spanning the deletions were run on an agarose gel to verify the presence of lower molecular weight bands indicating successful deletion formation. Expected deletion products: sg-Int39, 608 bp; sg-Int103, 544 bp; sg-Int153, 494 bp. FIG. 10C: Evaluation of RHO P347L mRNA intracellular levels by qPCR after transient transfection of 293TetO-RHO-P347L cells with wt SpCas9 and Guide 1 together with either sg-Int39, sg-Int103 or sg-Int153, as indicated. FIG. 10D: Evaluation of the intracellular levels of RHO P347L protein after transient transfection of 293TetO-RHO-P347L cells with wt SpCas9 and Guide 1 together with either sg-Int39, sg-Intl 03 or sg-Int153. In A and C data are presented as mean±SEM for n=2 biologically independent studies.

FIG. 11: Evaluation of Nme2Cas9 activity on the RHO P347L mutation. Indel formation at the mutant locus was evaluated after transient transfection of 293T-RHO-P347L clone 4 with Nme2Cas9 and different guide RNAs (Nme2Cas9 Guide 1- Guide 4), as indicated. For Guide 4 an alternative shorter sgRNA scaffold was evaluated in parallel to verify its editing efficiency.

### 6. DETAILED DESCRIPTION

The disclosure provides guide RNA (gRNA) molecules, which in combination with DNA endonucleases, e.g., Cas9 proteins, can be used, for example, to edit a human RHO gene having a P347 mutation, for example in a cell of a subject having a RHO gene with a P347 mutation.

In one aspect, a gRNA of the disclosure is engineered to comprise a spacer corresponding to a target domain in the genomic DNA sequence of a RHO gene having a P347 mutation. Typically, the target domain is adjacent to or near a protospacer-adjacent motif (PAM) of a Cas9 protein.

Exemplary features of gRNAs of the disclosure are described in Section 6.2. Exemplary Cas9 proteins which can be used in conjunction with gRNAs of the disclosure are described in Section 6.3.

The disclosure further provides nucleic acids encoding gRNAs of the disclosure, nucleic acids encoding Cas9 proteins, pluralities of nucleic acids and host cells containing the nucleic acids. Features of exemplary nucleic acids encoding gRNAs and Cas9 proteins and exemplary host cells are described in Section 6.4.

The disclosure further provides systems, particles, and cells containing gRNAs and nucleic acids of the disclosure. Exemplary systems, particles, and cells are described in Section 6.5.

The disclosure further provides pharmaceutical compositions comprising the gRNAs, nucleic acids, particles, and systems the disclosure. Exemplary pharmaceutical compositions are described in Section 6.6.

The disclosure further provides methods of using the gRNAs, nucleic acids, systems, particles, and pharmaceutical compositions of the disclosure for altering cells. Methods of the disclosure can be useful, for example, for treating a subject having RP caused a P347 mutation in the subject's RHO gene. Exemplary methods of altering cells are described in Section 6.7.

Those skilled in the relevant art will recognize and appreciate that many changes can be made to the various embodiments described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### 6.1. Definitions

Unless defined otherwise, all technical and scientific terms used herein have the same meaning as commonly understood to one of ordinary skill in the art to which this invention belongs. The following definitions are provided for the full understanding of terms used in this specification.

As used in the specification and claims, the singular form “a,” “an,” and “the” include plural references unless the context clearly dictates otherwise. For example, the term “an agent” includes a plurality of agents, including mixtures thereof.

A Cas9 protein refers to a wild-type or engineered Cas9 protein. Engineered Cas9 proteins can also be referred to as Cas9 variants. For the avoidance of doubt, any disclosure pertaining to a “Cas9” or “Cas9 protein” pertains to wild-type Cas9 proteins and Cas9 variants, unless the context dictates otherwise.

Identical or percent identity, in the context of two or more nucleic acids or polypeptide sequences, refer to two or more sequences or subsequences that are the same or have a specified percentage of amino acid residues or nucleotides that are the same as measured using a BLAST or BLAST 2.0 sequence comparison algorithms with default parameters described below, or by manual alignment and visual inspection (see, e.g., NCBI web site or the like). This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 15 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al., 1977, Nuc. Acids Res. 25:3389-3402, and Altschul et al., 1990, J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, 1993, Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

P347 mutation, in the context of this disclosure, refers to an alteration of a wild-type human RHO gene at the codon encoding amino acid P347. A P347 mutation can be, for example, a P347L mutation. As another example, a P347 mutation can be a P347S mutation. As another example, a P347 mutation can be a P347R mutation. As another example, a P347 mutation can be a P347Q mutation. As another example, a P347 mutation can be a P347T mutation. As another example, a P347 mutation can be a P347A mutation.

Peptide, protein, and polypeptide are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Polynucleotide and oligonucleotide are used interchangeably and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, primers and gRNAs. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (T) when the polynucleotide is RNA. Thus, the term “nucleotide sequence” is the alphabetical representation of a polynucleotide molecule.

Rhodopsin as used herein, refers to either Rhodopsin polypeptide, also known as opsin 2, OPN2, retinitis pigmentosa 4, CSNBAD1, and RP4, or a polynucleotide encoding Rhodopsin polypeptide. In humans, rhodopsin polypeptide is encoded by the RHO gene. In some embodiments, the Rhodopsin is a polypeptide or polynucleotide identified in one or more publicly available databases as follows: HGNC: 10012 Entrez Gene: 6010 Ensembl: ENSG00000163914 OMIM: 180380 UniProtKB: P08100. Table 1 shows exemplary rhodopsin sequences.

TABLE 1
Rhodopsin sequences

| | |
|---|---|
| Polypeptide | MNGTEGPNFYVPFSNATGVVRSPFEYPQYYLAEPWQFSMLAAYMFLLIVLGFPI |
| sequence | NFLTLYVTVQHKKLRTPLNYILLNLAVADLFMVLGGFTSTLYTSLHGYFVFGPTG |
| (amino acid | CNLEGFFATLGGEIALWSLVVLAIERYVVVCKPMSNFRFGENHAIMGVAFTWVM |
| P347 in bold | ALACAAPPLAGWSRYIPEGLQCSCGIDYYTLKPEVNNESFVIYMFVVHFTIPMIIIF |
| and boxed in) | FCYGQLVFTVKEAAAQQQESATTQKAEKEVTRMVIIMVIAFLICWVPYASVAFYIF |
| (SEQ ID | THQGSNFGPIFMTIPAFFAKSAAIYNPVIYIMMNKQFRNCMLTTICCGKNPLGDD |
| NO: 3) | 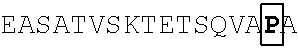 |
| Nucleotide | GGGGATTAATATGATTATGAACACCCCCAATCTCCCAGATGCTGATTC |
| sequence (the | AGCCAGGAGCTTAGGAGGGGGAGGTCACTTTATAAGGGTCTGGGGG |
| codon at | GGTCAGAACCCAGAGTCATCCAGCTGGAGCCCTGAGTGGCTGAGCT |
| position 6079- | CAGGCCTTCGCAGCATTCTTGGGTGGGAGCAGCCACGGGTCAGCCA |
| 6081 | CAAGGGCCACAGCCATGAATGGCACAGAAGGCCCTAACTTCTACGT |
| encoding | GCCCTTCTCCAATGCGACGGGTGTGGTACGCAGCCCCTTCGAGTAC |
| amino acid | CCACAGTACTACCTGGCTGAGCCATGGCAGTTCTCCATGCTGGCCG |
| P347 is in | CCTACATGTTTCTGCTGATCGTGCTGGGCTTCCCCATCAACTTCCTCA |
| bold and | CGCTCTACGTCACCGTCCAGCACAAGAAGCTGCGCACGCCTCTCAA |
| boxed in) | CTACATCCTGCTCAACCTAGCCGTGGCTGACCTCTTCATGGTCCTAG |
| (SEQ ID | GTGGCTTCACCAGCACCCTCTACACCTCTCTGCATGGATACTTCGTC |
| NO: 4) | TTCGGGCCCACAGGATGCAATTTGGAGGGCTTCTTTGCCACCCTGG |
| | GCGGTATGAGCCGGGTGTGGGTGGGGTGTGCAGGAGCCCGGGAGC |
| | ATGGAGGGGTCTGGGAGAGTCCCGGGCTTGGCGGTGGTGGCTGAG |
| | AGGCCTTCTCCCTTCTCCTGTCCTGTCAATGTTATCCAAAGCCCTCAT |
| | ATATTCAGTCAACAAACACCATTCATGGTGATAGCCGGGCTGCTGTTT |
| | GTGCAGGGCTGGCACTGAACACTGCCTTGATCTTATTTGGAGCAATA |
| | TGCGCTTGTCTAATTTCACAGCAAGAAAACTGAGCTGAGGCTCAAAG |
| | AAGTCAAGCGCCCTGCTGGGGCGTCACACAGGGACGGGTGCAGAG |
| | TTGAGTTGGAAGCCCGCATCTATCTCGGGCCATGTTTGCAGCACCAA |
| | GCCTCTGTTTCCCTTGGAGCAGCTGTGCTGAGTCAGACCCAGGCTG |
| | GGCACTGAGGGAGAGCTGGGCAAGCCAGACCCCTCCTCTCTGGGG |
| | GCCCAAGCTCAGGGTGGGAAGTGGATTTTCCATTCTCCAGTCATTGG |
| | GTCTTCCCTGTGCTGGGCAATGGGCTCGGTCCCCTCTGGCATCCTCT |
| | GCCTCCCCTCTCAGCCCCTGTCCTCAGGTGCCCCTCCAGCCTCCCT |
| | GCCGCGTTCCAAGTCTCCTGGTGTTGAGAACCGCAAGCAGCCGCTC |
| | TGAAGCAGTTCCTTTTTGCTTTAGAATAATGTCTTGCATTTAACAGGA |
| | AAACAGATGGGGTGCTGCAGGGATAACAGATCCCACTTAACAGAGA |
| | GGAAAACTGAGGCAGGGAGAGGGGAAGAGACTCATTTAGGGATGTG |
| | GCCAGGCAGCAACAAGAGCCTAGGTCTCCTGGCTGTGATCCAGGAA |
| | TATCTCTGCTGAGATGCAGGAGGAGACGCTAGAAGCAGCCATTGCAA |
| | AGCTGGGTGACGGGGAGAGCTTACCGCCAGCCACAAGCGTCTCTCT |
| | GCCAGCCTTGCCCTGTCTCCCCCATGTCCAGGCTGCTGCCTCGGTC |
| | CCATTCTCAGGGAATCTCTGGCCATTGTTGGGTGTTTGTTGCATTCAA |
| | TAATCACAGATCACTCAGTTCTGGCCAGAAGGTGGGTGTGCCACTTA |
| | CGGGTGGTTGTTCTCTGCAGGGTCAGTCCCAGTTTACAAATATTGTC |
| | CCTTTCACTGTTAGGAATGTCCCAGTTTGGTTGATTAACTATATGGCC |
| | ACTCTCCCTATGGAACTTCATGGGGTGGTGAGCAGGACAGATGTCTG |
| | AATTCCATCATTTCCTTCTTCTTCCTCTGGGCAAAACATTGCACATTG |
| | CTTCATGGCTCCTAGGAGAGGCCCCCACATGTCCGGGTTATTTCATT |
| | TCCCGAGAAGGGAGAGGGAGGAAGGACTGCCAATTCTGGGTTTCCA |
| | CCACCTCTGCATTCCTTCCCAACAAGGAACTCTGCCCCACATTAGGA |
| | TGCATTCTTCTGCTAAACACACACACACACACACACACACACAACACA |
| | CACACACACACACACACACACACACACACAAAACTCCCTACCGGGTT |
| | CCCAGTTCAATCCTGACCCCCTGATCTGATTCGTGTCCCTTATGGGC |
| | CCAGAGCGCTAAGCAAATAACTTCCCCCATTCCCTGGAATTTCTTTGC |
| | CCAGCTCTCCTCAGCGTGTGGTCCCTCTGCCCCTTCCCCCTCCTCCC |
| | AGCACCAAGCTCTCTCCTTCCCCAAGGCCTCCTCAAATCCCTCTCCC |
| | ACTCCTGGTTGCCTTCCTAGCTACCCTCTCCCTGTCTAGGGGGGAGT |
| | GCACCCTCCTTAGGCAGTGGGGTCTGTGCTGACCGCCTGCTGACTG |
| | CCTTGCAGGTGAAATTGCCCTGTGGTCCTTGGTGGTCCTGGCCATCG |
| | AGCGGTACGTGGTGGTGTGTAAGCCCATGAGCAACTTCCGCTTCGG |
| | GGAGAACCATGCCATCATGGGCGTTGCCTTCACCTGGGTCATGGCG |
| | CTGGCCTGCGCCGCACCCCCACTCGCCGGCTGGTCCAGGTAATGGC |
| | ACTGAGCAGAAGGGAAGAAGCTCCGGGGGCTCTTTGTAGGGTCCTC |
| | CAGTCAGGACTCAAACCCAGTAGTGTCTGGTTCCAGGCACTGACCTT |
| | GTATGTCTCCTGGCCCAAATGCCCACTCAGGGTAGGGGTGTAGGGC |
| | AGAAGAAGAAACAGACTCTAATGTTGCTACAAGGGCTGGTCCCATCT |
| | CCTGAGCCCCATGTCAAACAGAATCCAAGACATCCCAACCCTTCACC |
| | TTGGCTGTGCCCCTAATCCTCAACTAAGCTAGGCGCAAATTCCAATC |
| | CTCTTTGGTCTAGTACCCCGGGGGCAGCCCCCTCTAACCTTGGGCC |
| | TCAGCAGCAGGGGAGGCCACACCTTCCTAGTGCAGGTGGCCATATT |
| | GTGGCCCCTTGGAACTGGGTCCCACTCAGCCTCTAGGCGATTGTCT |
| | CCTAATGGGGCTGAGATGAGACACAGTGGGGACAGTGGTTTGGACA |
| | ATAGGACTGGTGACTCTGGTCCCCAGAGGCCTCATGTCCCTCTGTCT |
| | CCAGAAAATTCCCACTCTCACTTCCCTTTCCTCCTCAGTCTTGCTAGG |
| | GTCCATTTCTTACCCCTTGCTGAATTTGAGCCCACCCCCTGGACTTTT |
| | TCCCCATCTTCTCCAATCTGGCCTAGTTCTATCCTCTGGAAGCAGAG |
| | CCGCTGGACGCTCTGGGTTTCCTGAGGCCCGTCCACTGTCACCAAT |
| | ATCAGGAACCATTGCCACGTCCTAATGACGTGCGCTGGAAGCCTCTA |
| | GTTTCCAGAAGCTGCACAAAGATCCCTTAGATACTCTGTGTGTCCATC |
| | TTTGGCCTGGAAAATACTCTCACCCTGGGGCTAGGAAGACCTCGGTT |
| | TGTACAAACTTCCTCAAATGCAGAGCCTGAGGGCTCTCCCCACCTCC |
| | TCACCAACCCTCTGCGTGGCATAGCCCTAGCCTCAGCGGGCAGTGG |
| | ATGCTGGGGCTGGGCATGCAGGGAGAGGCTGGGTGGTGTCATCTG |
| | GTAACGCAGCCACCAAACAATGAAGCGACACTGATTCCACAAGGTGC |
| | ATCTGCATCCCCATCTGATCCATTCCATCCTGTCACCCAGCCATGCA |
| | GACGTTTATGATCCCCTTTTCCAGGGAGGGAATGTGAAGCCCCAGAA |
| | AGGGCCAGCGCTCGGCAGCCACCTTGGCTGTTCCCAAGTCCCTCAC |
| | AGGCAGGGTCTCCCTACCTGCCTGTCCTCAGGTACATCCCCGAGGG |
| | CCTGCAGTGCTCGTGTGGAATCGACTACTACACGCTCAAGCCGGAG |
| | GTCAACAACGAGTCTTTTGTCATCTACATGTTCGTGGTCCACTTCACC |
| | ATCCCCATGATTATCATCTTTTTCTGCTATGGGCAGCTCGTCTTCACC |
| | GTCAAGGAGGTACGGGCCGGGGGGTGGGCGGCCTCACGGCTCTGA |
| | GGGTCCAGCCCCCAGCATGCATCTGCGGCTCCTGCTCCCTGGAGGA |
| | GCCATGGTCTGGACCCGGGTCCCGTGTCCTGCAGGCCGCTGCCCA |
| | GCAGCAGGAGTCAGCCACCACACAGAAGGCAGAGAAGGAGGTCACC |
| | CGCATGGTCATCATCATGGTCATCGCTTTCCTGATCTGCTGGGTGCC |
| | CTACGCCAGCGTGGCATTCTACATCTTCACCCACCAGGGCTCCAACT |
| | TCGGTCCCATCTTCATGACCATCCCAGCGTTCTTTGCCAAGAGCGCC |
| | GCCATCTACAACCCTGTCATCTATATCATGATGAACAAGCAGGTGCC |
| | TACTGCGGGTGGGAGGGCCCCAGTGCCCCAGGCCACAGGCGCTGC |
| | CTGCCAAGGACAAGCTACTTCCCAGGGCAGGGGAGGGGGCTCCATC |
| | AGGGTTACTGGCAGCAGTCTTGGGTCAGCAGTCCCAATGGGGAGTG |
| | TGTGAGAAATGCAGATTCCTGGCCCCACTCAGAACTGCTGAATCTCA |
| | GGGTGGGCCCAGGAACCTGCATTTCCAGCAAGCCCTCCACAGGTGG |
| | CTCAGATGCTCACTCAGGTGGGAGAAGCTCCAGTCAGCTAGTTCTGG |
| | AAGCCCAATGTCAAAGTCAGAAGGACCCAAGTCGGGAATGGGATGG |
| | GCCAGTCTCCATAAAGCTGAATAAGGAGCTAAAAAGTCTTATTCTGAG |
| | GGGTAAAGGGGTAAAGGGTTCCTCGGAGAGGTACCTCCGAGGGGTA |
| | AACAGTTGGGTAAACAGTCTCTGAAGTCAGCTCTGCCATTTTCTAGCT |
| | GTATGGCCCTGGGCAAGTCAATTTCCTTCTCTGTGCTTTGGTTTCCTC |
| | ATCCATAGAAAGGTAGAAAGGGCAAAACACCAAACTCTTGGATTACA |
| | AGAGATAATTTACAGAACACCCTTGGCACACAGAGGGCACCATGAAA |
| | TGTCACGGGTGACACAGCCCCCTTGTGCTCAGTCCCTGGCATCTCTA |
| | GGGGTGAGGAGCGTCTGCCTAGCAGGTTCCCTCCAGGAAGCTGGAT |
| | TTGAGTGGATGGGGCGCTGGAATCGTGAGGGGCAGAAGCAGGCAAA |
| | GGGTCGGGGCGAACCTCACTAACGTGCCAGTTCCAAGCACACTGTG |
| | GGCAGCCCTGGCCCTGACTCAAGCCTCTTGCCTTCCAGTTCCGGAA |
| | CTGCATGCTCACCACCATCTGCTGCGGCAAGAACCCACTGGGTGAC |
| | GATGAGGCCTCTGCTACCGTGTCCAAGACGGAGACGAGCCAGGTGG |
| | 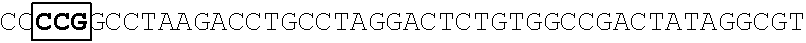 |
| | CTCCCATCCCCTACACCTTCCCCCAGCCACAGCCATCCCACCAGGA |
| | GCAGCGCCTGTGCAGAATGAACGAAGTCACATAGGCTCCTTAATTT |
| | TTTTTTTTTTTTIAAGAAATAATTAATGAGGCTCCTCACTCACCTGGGAC |
| | AGCCTGAGAAGGGACATCCACCA |

Spacer refers to a region of a gRNA molecule which is partially or fully complementary to a target sequence found in the + or − strand of a RHO genomic DNA. When complexed with a DNA endonuclease such as a Cas9 protein, the gRNA directs the DNA endonuclease to the target sequence in the genomic DNA. A spacer of a Cas9 gRNA is typically 15 to 30 nucleotides in length (e.g., 20-25 nucleotides). The nucleotide sequence of a spacer can be, but is not necessarily, fully complementary to the target sequence. For example, a spacer can contain one or more mismatches with a target sequence, e.g., the spacer can comprise one, two, or three mismatches with the target sequence.

The terms treat, treating, treatment, and grammatical variations thereof as used herein, include partially or completely delaying, curing, healing, alleviating, relieving, altering, remedying, ameliorating, improving, stabilizing, mitigating, and/or reducing the intensity or frequency of one or more diseases or conditions, symptoms of a disease or condition, or underlying causes of a disease or condition. Treatments according to the disclosure may be applied prophylactically, palliatively or remedially. Prophylactic treatments can be administered to a subject prior to onset, during early onset (e.g., upon initial signs and symptoms of RP), or after an established development of RP. Prophylactic administration can occur for several days to years prior to the manifestation of symptoms.

In some instances, the terms treat, treating, treatment and grammatical variations thereof, include reducing expression of a P347 mutant rhodopsin (RHO) gene. The terms treat, treating, treatment and grammatical variations thereof, can also include reducing RHO protein misfolding and/or mislocalization in retinal cells, e.g., epithelial cells. The terms treat, treating, treatment and grammatical variations thereof, can also include decreasing retinal epithelial cell death and/or retinal degeneration. The terms treat, treating, treatment and grammatical variations thereof, can also include increasing a ratio of expression of a wild-type rhodopsin allele to a rhodopsin P347 mutant allele. Measurements of treatment can be compared with prior treatment(s) of the subject, inclusive of no treatment, or compared with the incidence of such symptom(s) in a general or study population.

Wild-type, in reference to a genomic DNA sequence, refers to a genomic DNA sequence that predominates in a species, e.g., Homo sapiens.

### 6.2. Guide RNA Molecules

The disclosure provides gRNA molecules that can be used with CRISPR (Clustered Regularly Interspaced Short Palindromic Repeats) endonucleases to edit a human RHO gene having a P347 mutation. A review of CRISPR endonuclease systems, including Type II and Type V CRISPR systems is described in WO 2019/102381, the contents of which are incorporated herein by reference in their entireties.

gRNAs of the disclosure are typically Cas9 gRNAs and comprise a spacer of 15 to 30 nucleotides in length in length. gRNAs of the disclosure are in some embodiments single guide RNAs (sgRNAs), which typically comprise the spacer at the 5′ end of the molecule and a 3′ sgRNA segment. Further features of exemplary gRNA spacer sequences are described in Section 6.2.1 and further features of exemplary 3′ sgRNA segments are described in Section 6.2.2.

6.2.1. Spacers

The spacer sequence is partially or fully complementary to a target sequence found in a human RHO gene having a P347 mutation. For example, a 20 nucleotide spacer sequence can be partially or fully complementary to a 20 nucleotide sequence in the RHO gene. A spacer that is partially complementary to a target sequence can have, for example, one, two, or three mismatches with the target sequence.

DNA endonucleases such as Cas9 require a specific sequence, called a protospacer adjacent motif (PAM) that is downstream (e.g., directly downstream) of the target sequence on the non-target strand. Wild-type S. pyogenes Cas9 recognizes a PAM sequence of NGG that is found downstream of the target sequence in the genomic DNA on the non-target strand, wherein “N” refers to any nucleotide. Nme2Cas9 recognizes a NNNNCC PAM sequence that is found downstream of the target sequence in the genomic DNA on the non-target strand. Figure. 5 of Fonfara et al., 2014, Nucleic Acids Research, 42:2577-2590 provides PAM sequences for the Cas9 proteins from various species. In some embodiments, the spacer sequences of the gRNAs of the disclosure are complementary to a target sequence that is adjacent to a PAM, for example NGG on the non-target strand.

gRNAs of the disclosure can comprise a spacer that is 15 to 30 nucleotides in length (e.g., 15 to 25, 16 to 24, 17 to 23, 18 to 22, 19 to 21, 18 to 30, 20 to 28, 22 to 26, or 23 to 25 nucleotides in length). In some embodiments, a spacer is 15 nucleotides in length. In other embodiments, a spacer is 16 nucleotides in length. In other embodiments, a spacer is 17 nucleotides in length. In other embodiments, a spacer is 18 nucleotides in length. In other embodiments, a spacer is 19 nucleotides in length. In other embodiments, a spacer is 20 nucleotides in length. In other embodiments, a spacer is 21 nucleotides in length. In other embodiments, a spacer is 22 nucleotides in length. In other embodiments, a spacer is 23 nucleotides in length. In other embodiments, a spacer is 24 nucleotides in length. In other embodiments, a spacer is 25 nucleotides in length. In other embodiments, a spacer is 26 nucleotides in length. In other embodiments, a spacer is 27 nucleotides in length. In other embodiments, a spacer is 28 nucleotides in length. In other embodiments, a spacer is 29 nucleotides in length. In other embodiments, a spacer is 30 nucleotides in length.

The target sequence can, but need not necessarily, include the nucleotide(s) causing the P347 mutation. By selecting a spacer having a nucleotide sequence that does not have a mismatch with the target sequence at the position(s) corresponding to the P347 mutation, a gRNA complexed with a DNA endonuclease such as Cas9 can have selectivity for the mutant allele, resulting in preferential editing of the mutant allele. Without being bound by theory, it is believed that by this mechanism, the DNA endonuclease can preferentially cleave the mutant RHO allele comprising the P347 mutation (resulting in editing of the mutant allele during repair of the cleaved DNA), while the wild-type RHO allele remains intact to express wild-type rhodopsin. Table 2A lists exemplary spacer sequences that can be used to preferentially target a RHO gene having a P347L mutation. Spacers 1-3 in Table 2A can be used, for example, in SpCas9 gRNAs, and spacers 4-7 in Table 2A can be used, for example, in Nme2Cas9 gRNAs. In Table 2A, the nucleotide corresponding to a mutation causing the P347L mutation is shown in bold text. Lowercase nucleotides in Table 2A indicate a mismatch with the wild-type genomic RHO sequence.

TABLE 2A
Spacer Sequences

| | | SEQ |
|---|---|---|
| Spacer | RNA Sequence | ID NO |
| 1 | GGUCUUAGGCCAGGGCCACC | 5 |
| 2 | GCCCUGGCCUAAGACCUGCCU | 6 |
| 3 | CCUAGGCAGGUCUUAGGCCA | 7 |
| 3 (with 5′ | gCCUAGGCAGGUCUUAGGCCA | 8 |
| mismatch) | | |
| 4 | GACGAGCCAGGUGGCCCUGGCCU | 9 |
| 5 | GUCCUAGGCAGGUCUUAGGCCAGG | 10 |
| 6 | UUAGGCCAGGGCCACCUGGCUC | 11 |
| 7 | GCCAGGUGGCCCUGGCCUAAGA | 12 |

The spacer sequences set forth in Table 2A can be modified for targeting a RHO gene having a P347 mutation other than P347L, for example P347S, P347R, P347Q, P347T, or P347A, by replacing the nucleotides corresponding to the P347L mutation with nucleotides corresponding to another P347 mutation. For example, by replacing the “U” shown in bold text in spacer 2 (which corresponds to the P347L mutation of the wild-type proline codon CCG to the leucine codon CTG) with “G”, a spacer for targeting a P347R mutation can be made (mutation of the wild-type CCG proline codon to the CGG arginine codon results in a P347R mutation).

In some embodiments, a gRNA of the disclosure has a spacer whose nucleotide sequence comprises 15 or more consecutive nucleotides from a sequence shown in Table 2A. In some embodiments, a gRNA of the disclosure has a spacer whose nucleotide sequence comprises 16 or more consecutive nucleotides from a sequence shown in Table 2A. In some embodiments, a gRNA of the disclosure has a spacer whose nucleotide sequence comprises 17 or more consecutive nucleotides from a sequence shown in Table 2A. In some embodiments, a gRNA of the disclosure has a spacer whose nucleotide sequence comprises 18 or more consecutive nucleotides from a sequence shown in Table 2A. In some embodiments, a gRNA of the disclosure has a spacer whose nucleotide sequence comprises 19 or more consecutive nucleotides from a sequence shown in Table 2A. In some embodiments, a gRNA of the disclosure has a spacer whose nucleotide sequence comprises 20 or more consecutive nucleotides from a sequence shown in Table 2A. In some embodiments, a gRNA of the disclosure has a spacer whose nucleotide sequence comprises 21 or more consecutive nucleotides from a sequence shown in Table 2A. In some embodiments, a gRNA of the disclosure has a spacer whose nucleotide sequence comprises 22 or more consecutive nucleotides from a sequence shown in Table 2A. In some embodiments, a gRNA of the disclosure has a spacer whose nucleotide sequence comprises 23 or more consecutive nucleotides from a sequence shown in Table 2A. In some embodiments, a gRNA of the disclosure has a spacer whose nucleotide sequence comprises 24 consecutive nucleotides from a sequence shown in Table 2A.

In some embodiments, a gRNA of the disclosure has a spacer whose nucleotide sequence is at least 85% identical to a spacer sequence shown in Table 2A. In some embodiments, a gRNA of the disclosure has a spacer whose nucleotide sequence is at least 90% identical to a spacer sequence shown in Table 2A. In some embodiments, a gRNA of the disclosure has a spacer whose nucleotide sequence is at least 95% identical to a spacer sequence shown in Table 2A. In some embodiments, a gRNA of the disclosure has a spacer whose nucleotide sequence is identical to a spacer sequence shown in Table 2A.

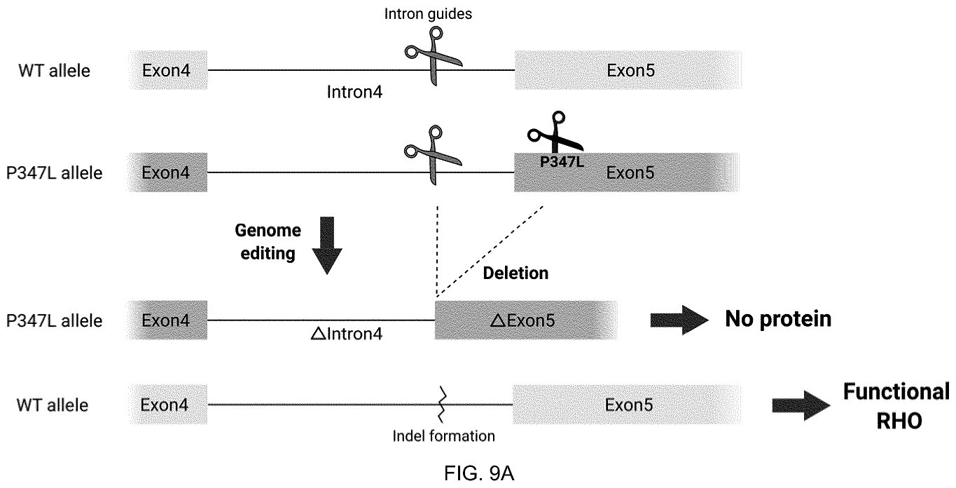

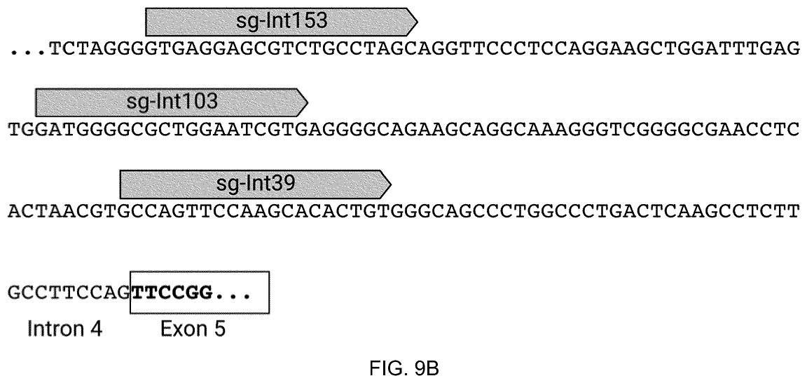

gRNAs having a spacer sequence that is partially or fully complementary to a target sequence found in a human RHO gene having a P347 mutation can be used together with a gRNA having a spacer sequence that is partially or fully complementary to a target sequence in intron 4 of a human RHO gene. Using a combination of gRNAs can promote deletion within a human RHO gene having a P347 mutation while preserving the functionality of the wild-type allele (see, Example 3 and FIG. 9A). Table 2B shows genomic sequences in intron 4 of the RHO gene that can be used to make an intron 4 targeting gRNA. In some embodiments, the spacer sequence comprises the spacer sequence of sg-Int39, sg-Int103, or sg-Int153 (see, Example 3; Table 13).

TABLE 2B
RHO Intron 4 Genomic Sequences

| Target | | SEQ ID |
|---|---|---|
| name | Genomic Sequence | NO: |
| 416forw | TCCTCGGAGAGGTACCTCCGAGG | 13 |
| 417forw | CCTCGGAGAGGTACCTCCGAGGG | 14 |
| 410rev | CCCAACTGTTTACCCCTCGGAGG | 15 |
| 418forw | CTCGGAGAGGTACCTCCGAGGGG | 16 |
| 735forw | TGGGGCGCTGGAATCGTGAGGGG | 17 |
| 765rev | TTGGAACTGGCACGTTAGTGAGG | 18 |
| 308rev | GCCCATCCCATTCCCGACTTGGG | 19 |
| 322forw | GAAGGACCCAAGTCGGGAATGGG | 20 |
| 430forw | CCTCCGAGGGGTAAACAGTTGGG | 21 |
| 309rev | GGCCCATCCCATTCCCGACTTGG | 22 |
| 734forw | ATGGGGCGCTGGAATCGTGAGGG | 23 |
| 429forw | ACCTCCGAGGGGTAAACAGTTGG | 24 |
| 733forw | GATGGGGCGCTGGAATCGTGAGG | 25 |
| 405forw | GGGTAAAGGGTTCCTCGGAGAGG | 26 |
| 397rev | CCCTCGGAGGTACCTCTCCGAGG | 27 |
| 265rev | GGCTTCCAGAACTAGCTGACTGG | 28 |
| 321forw | AGAAGGACCCAAGTCGGGAATGG | 29 |
| 135forw | CTTGGGTCAGCAGTCCCAATGGG | 30 |
| 605rev | GTGTCACCCGTGACATTTCATGG | 31 |
| 136forw | TTGGGTCAGCAGTCCCAATGGGG | 32 |
| 280forw | AAGCTCCAGTCAGCTAGTTCTGG | 33 |
| 413rev | TTACCCAACTGTTTACCCCTCGG | 34 |
| 4rev | GGGGCCCTCCCACCCGCAGTAGG | 35 |
| 105forw | GGGGGCTCCATCAGGGTTACTGG | 36 |
| 683forw | GTGAGGAGCGTCTGCCTAGCAGG | 37 |
| 723forw | GATTTGAGTGGATGGGGCGCTGG | 38 |
| 118forw | GGGTTACTGGCAGCAGTCTTGGG | 39 |
| 326forw | GACCCAAGTCGGGAATGGGATGG | 40 |
| 647rev | TCCTCACCCCTAGAGATGCCAGG | 41 |
| 589forw | GATAATTTACAGAACACCCTTGG | 42 |
| 327forw | ACCCAAGTCGGGAATGGGATGGG | 43 |
| 383forw | AGTCTTATTCTGAGGGGTAAAGG | 44 |
| 134forw | TCTTGGGTCAGCAGTCCCAATGG | 45 |
| 287rev | GGTCCTTCTGACTTTGACATTGG | 46 |
| 50rev | GGAAGTAGCTTGTCCTTGGCAGG | 47 |
| 117forw | AGGGTTACTGGCAGCAGTCTTGG | 48 |
| 352forw | AGTCTCCATAAAGCTGAATAAGG | 49 |
| 400forw | TAAAGGGGTAAAGGGTTCCTCGG | 50 |
| 758forw | CAGAAGCAGGCAAAGGGTCGGGG | 51 |
| 618forw | GAGGGCACCATGAAATGTCACGG | 52 |
| 646rev | CCTCACCCCTAGAGATGCCAGGG | 53 |
| 796forw | TGCCAGTTCCAAGCACACTGTGG | 54 |
| 92rev | ACTGCTGCCAGTAACCCTGATGG | 55 |
| 230forw | ATTTCCAGCAAGCCCTCCACAGG | 56 |
| 337rev | TAGCTCCTTATTCAGCTTTATGG | 57 |
| 660forw | CTCAGTCCCTGGCATCTCTAGGG | 58 |
| 661forw | TCAGTCCCTGGCATCTCTAGGGG | 59 |
| 716forw | GAAGCTGGATTTGAGTGGATGGG | 60 |
| 129rev | TTTCTCACACACTCCCCATTGGG | 61 |
| 233forw | TCCAGCAAGCCCTCCACAGGTGG | 62 |
| 251forw | GGTGGCTCAGATGCTCACTCAGG | 63 |
| 75forw | CAAGGACAAGCTACTTCCCAGGG | 64 |
| 214rev | GCCACCTGTGGAGGGCTTGCTGG | 65 |
| 384forw | GTCTTATTCTGAGGGGTAAAGGG | 66 |
| 80forw | ACAAGCTACTTCCCAGGGCAGGG | 67 |
| 255forw | GCTCAGATGCTCACTCAGGTGGG | 68 |
| 315forw | AAAGTCAGAAGGACCCAAGTCGG | 69 |
| 79forw | GACAAGCTACTTCCCAGGGCAGG | 70 |
| 548rev | TCTCTTGTAATCCAAGAGTTTGG | 71 |
| 54rev | CCTGGGAAGTAGCTTGTCCTTGG | 72 |
| 223rev | AGCATCTGAGCCACCTGTGGAGG | 73 |
| 286rev | GTCCTTCTGACTTTGACATTGGG | 74 |
| 376forw | GCTAAAAAGTCTTATTCTGAGGG | 75 |
| 385forw | TCTTATTCTGAGGGGTAAAGGGG | 76 |
| 38rev | TCCTTGGCAGGCAGCGCCTGTGG | 77 |
| 130rev | ATTTCTCACACACTCCCCATTGG | 78 |
| 585rev | TGGTGCCCTCTGTGTGCCAAGGG | 79 |
| 619forw | AGGGCACCATGAAATGTCACGGG | 80 |
| 304forw | AGCCCAATGTCAAAGTCAGAAGG | 81 |
| 391forw | TCTGAGGGGTAAAGGGGTAAAGG | 82 |
| 600forw | GAACACCCTTGGCACACAGAGGG | 83 |
| 659forw | GCTCAGTCCCTGGCATCTCTAGG | 84 |
| 97forw | GCAGGGGAGGGGGCTCCATCAGG | 85 |
| 666forw | CCCTGGCATCTCTAGGGGTGAGG | 86 |
| 778rev | GCCCACAGTGTGCTTGGAACTGG | 87 |
| 797forw | GCCAGTTCCAAGCACACTGTGGG | 88 |
| 537forw | TCATCCATAGAAAGGTAGAAAGG | 89 |
| 694forw | CTGCCTAGCAGGTTCCCTCCAGG | 90 |
| 74forw | CCAAGGACAAGCTACTTCCCAGG | 91 |
| 98forw | CAGGGGAGGGGGCTCCATCAGGG | 92 |
| 194forw | GAACTGCTGAATCTCAGGGTGGG | 93 |
| 254forw | GGCTCAGATGCTCACTCAGGTGG | 94 |
| 330rev | TTATTCAGCTTTATGGAGACTGG | 95 |
| 557forw | AGGGCAAAACACCAAACTCTTGG | 96 |
| 193forw | AGAACTGCTGAATCTCAGGGTGG | 97 |
| 200forw | CTGAATCTCAGGGTGGGCCCAGG | 98 |
| 375forw | AGCTAAAAAGTCTTATTCTGAGG | 99 |
| 515rev | TTTCTACCTTTCTATGGATGAGG | 100 |
| 631rev | TGCCAGGGACTGAGCACAAGGGG | 101 |
| 701forw | GCAGGTTCCCTCCAGGAAGCTGG | 102 |
| 717forw | AAGCTGGATTTGAGTGGATGGGG | 103 |
| 190forw | CTCAGAACTGCTGAATCTCAGGG | 104 |
| 226rev | GTGAGCATCTGAGCCACCTGTGG | 105 |
| 599forw | AGAACACCCTTGGCACACAGAGG | 106 |
| 711forw | TCCAGGAAGCTGGATTTGAGTGG | 107 |
| 745forw | GAATCGTGAGGGGCAGAAGCAGG | 108 |
| 81forw | CAAGCTACTTCCCAGGGCAGGGG | 109 |
| 632rev | ATGCCAGGGACTGAGCACAAGGG | 110 |
| 688rev | CTCAAATCCAGCTTCCTGGAGGG | 111 |
| 757forw | GCAGAAGCAGGCAAAGGGTCGGG | 112 |
| 586rev | ATGGTGCCCTCTGTGTGCCAAGG | 113 |
| 715forw | GGAAGCTGGATTTGAGTGGATGG | 114 |
| 806forw | AAGCACACTGTGGGCAGCCCTGG | 115 |
| 810rev | GAAGGCAAGAGGCTTGAGTCAGG | 116 |
| 57forw | GCCACAGGCGCTGCCTGCCAAGG | 117 |
| 756forw | GGCAGAAGCAGGCAAAGGGTCGG | 118 |
| 784rev | AGGGCTGCCCACAGTGTGCTTGG | 119 |
| 689rev | ACTCAAATCCAGCTTCCTGGAGG | 120 |
| 24rev | CGCCTGTGGCCTGGGGCACTGGG | 121 |
| 189forw | ACTCAGAACTGCTGAATCTCAGG | 122 |
| 32rev | GCAGGCAGCGCCTGTGGCCTGGG | 123 |
| 677rev | CTTCCTGGAGGGAACCTGCTAGG | 124 |
| 33rev | GGCAGGCAGCGCCTGTGGCCTGG | 125 |
| 167rev | TGAGATTCAGCAGTTCTGAGTGG | 126 |
| 377forw | CTAAAAAGTCTTATTCTGAGGGG | 127 |
| 521rev | TTGCCCTTTCTACCTTTCTATGG | 128 |
| 166rev | GAGATTCAGCAGTTCTGAGTGGG | 129 |
| 809rev | AAGGCAAGAGGCTTGAGTCAGGG | 130 |
| 804rev | AAGAGGCTTGAGTCAGGGCCAGG | 131 |
| 165rev | AGATTCAGCAGTTCTGAGTGGGG | 132 |
| 31rev | CAGGCAGCGCCTGTGGCCTGGGG | 133 |
| 205rev | GGAGGGCTTGCTGGAAATGCAGG | 134 |
| 803rev | AGAGGCTTGAGTCAGGGCCAGGG | 135 |
| 42forw | GCCCCAGTGCCCCAGGCCACAGG | 136 |
| 751forw | TGAGGGGCAGAAGCAGGCAAAGG | 137 |
| 84forw | GCTACTTCCCAGGGCAGGGGAGG | 138 |
| 222rev | GCATCTGAGCCACCTGTGGAGGG | 139 |
| 25rev | GCGCCTGTGGCCTGGGGCACTGG | 140 |
| 85forw | CTACTTCCCAGGGCAGGGGAGGG | 141 |
| 630rev | GCCAGGGACTGAGCACAAGGGGG | 142 |
| 86forw | TACTTCCCAGGGCAGGGGAGGGG | 143 |
| 160rev | CAGCAGTTCTGAGTGGGGCCAGG | 144 |
| 392forw | CTGAGGGGTAAAGGGGTAAAGGG | 145 |
| 198rev | TTGCTGGAAATGCAGGTTCCTGG | 146 |
| 538forw | CATCCATAGAAAGGTAGAAAGGG | 147 |
| 23rev | GCCTGTGGCCTGGGGCACTGGGG | 148 |
| 633rev | GATGCCAGGGACTGAGCACAAGG | 149 |
| 35forw | TGGGAGGGCCCCAGTGCCCCAGG | 150 |
| 197rev | TGCTGGAAATGCAGGTTCCTGGG | 151 |
| 752forw | GAGGGGCAGAAGCAGGCAAAGGG | 152 |
| 649forw | GCCCCCTTGTGCTCAGTCCCTGG | 153 |
| 87forw | ACTTCCCAGGGCAGGGGAGGGGG | 154 |
| 509forw | CAATTTCCTTCTCTGTGCTTTGG | 155 |
| 72rev | TGGAGCCCCCTCCCCTGCCCTGG | 156 |
| 316forw | AAGTCAGAAGGACCCAAGTCGGG | 157 |
| 480forw | ATTTTCTAGCTGTATGGCCCTGG | 158 |
| 481forw | TTTTCTAGCTGTATGGCCCTGGG | 159 |
| 71rev | GGAGCCCCCTCCCCTGCCCTGGG | 160 |
| 458rev | AGGGCCATACAGCTAGAAAATGG | 161 |
| 474forw | TCTGCCATTTTCTAGCTGTATGG | 162 |
| 692rev | TCCACTCAAATCCAGCTTCCTGG | 163 |
| 478rev | AGAAGGAAATTGACTTGCCCAGG | 164 |
| 162forw | GTGTGAGAAATGCAGATTCCTGG | 165 |
| 529forw | TGGTTTCCTCATCCATAGAAAGG | 166 |
| 477rev | GAAGGAAATTGACTTGCCCAGGG | 167 |
| 495rev | AGGAAACCAAAGCACAGAGAAGG | 168 |

6.2.2. sgRNA Molecules

gRNAs of the disclosure can be single-guide RNA (sgRNA) molecules. A sgRNA in a Type II CRISPR system can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

The sgRNA can comprise a variable length spacer sequence (e.g., 15 to 30 nucleotides) at the 5′ end of the sgRNA sequence and a 3′ sgRNA segment. Various 3′ sgRNA segments are known in the art. Exemplary 3′ sgRNA sequences for SpCas9 sgRNAs are shown in Table 3. Exemplary 3′ sgRNA sequences for Nme2Cas9 sgRNAs are shown in Table 4.

TABLE 3
SpCas9 3′ sgRNA sequences

| 3′ sgRNA | | SEQ |
|---|---|---|
| sequence | Sequence | ID NO |
| 1 | GUUUUAGAGCUAGAAAUAGCAAGUUAAAAUAAGGCUAGUCCGUU | 169 |
| | AUCAACUUGAAAAAGUGGCACCGAGUCGGUGCUUUUUU | |
| 2 | GUUUUAGAGCUAGAAAUAGCAAGUUAAAAUAAGGCUAGUCCGUU | 170 |
| | AUCAACUUGAAAAAGUGGCACCGAGUCGGUGCUUUUU | |
| 3 | GUUUUAGAGCUAGAAAUAGCAAGUUAAAAUAAGGCUAGUCCGUU | 171 |
| | AUCAACUUGAAAAAGUGGCACCGAGUCGGUGCUUUU | |
| 4 | GUUUUAGAGCUAGAAAUAGCAAGUUAAAAUAAGGCUAGUCCGUU | 172 |
| | AUCAACUUGAAAAAGUGGCACCGAGUCGGUGCUUU | |
| 5 | GUUUUAGAGCUAGAAAUAGCAAGUUAAAAUAAGGCUAGUCCGUU | 173 |
| | AUCAACUUGAAAAAGUGGCACCGAGUCGGUGCUU | |
| 6 | GUUUUAGAGCUAGAAAUAGCAAGUUAAAAUAAGGCUAGUCCGUU | 174 |
| | AUCAACUUGAAAAAGUGGCACCGAGUCGGUGCU | |
| 7 | GUUUUAGAGCUAGAAAUAGCAAGUUAAAAUAAGGCUAGUCCGUU | 175 |
| | AUCAACUUGAAAAAGUGGCACCGAGUCGGUGC | |
| 8 | GUUUAAGAGCUAUGCUGGAAACAGCAUAGCAAGUUUAAAUAAGG | 176 |
| | CUAGUCCGUUAUCAACUUGAAAAAGUGGCACCGAGUCGGUGCU | |
| | UUUUU | |
| 9 | GUUUAAGAGCUAUGCUGGAAACAGCAUAGCAAGUUUAAAUAAGG | 177 |
| | CUAGUCCGUUAUCAACUUGAAAAAGUGGCACCGAGUCGGUGCU | |
| | UUUU | |
| 10 | GUUUAAGAGCUAUGCUGGAAACAGCAUAGCAAGUUUAAAUAAGG | 178 |
| | CUAGUCCGUUAUCAACUUGAAAAAGUGGCACCGAGUCGGUGCU | |
| | UUU | |
| 11 | GUUUAAGAGCUAUGCUGGAAACAGCAUAGCAAGUUUAAAUAAGG | 179 |
| | CUAGUCCGUUAUCAACUUGAAAAAGUGGCACCGAGUCGGUGCU | |
| | UU | |
| 12 | GUUUAAGAGCUAUGCUGGAAACAGCAUAGCAAGUUUAAAUAAGG | 180 |
| | CUAGUCCGUUAUCAACUUGAAAAAGUGGCACCGAGUCGGUGCU | |
| | U | |
| 13 | GUUUAAGAGCUAUGCUGGAAACAGCAUAGCAAGUUUAAAUAAGG | 181 |
| | CUAGUCCGUUAUCAACUUGAAAAAGUGGCACCGAGUCGGUGCU | |
| 14 | GUUUAAGAGCUAUGCUGGAAACAGCAUAGCAAGUUUAAAUAAGG | 182 |
| | CUAGUCCGUUAUCAACUUGAAAAAGUGGCACCGAGUCGGUGC | |
| 15 | GUUUUAGAGCUAGAAAUAGCAAGUUAAAAUAAGGCUAGUCCGUU | 183 |
| | AUCAACUUGAAAAAGUGGCACCGAGUCGGUGCUUUUUUUU | |
| 16 | GUUUAAGUACUCUGUGCUGGAAACAGCACAGAAUCUACUUAAAC | 184 |
| | AAGGCAAAAUGCCGUGUUUAUCUCGUCAACUUGUUGGCGAGAU | |
| | UUU | |
| 17 | GUUUAAGUACUCUGUGCUGGAAACAGCACAGAAUCUACUUAAAC | 185 |
| | AAGGCAAAAUGCCGUGUUUAUCUCGUCAACUUGUUGGCGAGA | |
| 18 | GUUUAAGUACUCUGUGCUGGAAACAGCACAGAAUCUACUUAAAC | 186 |
| | AAGGCAAAAUGCCGUGUUUAUCUCGUCAACUUGUUGGCGAGAU | |
| | UUUUUUU | |
| 19 | GUUUUAGUACUCUGUAAUGAAAAUUACAGAAUCUACUAAAACAA | 187 |
| | GGCAAAAUGCCGUGUUUAUCUCGUCAACUUGUUGGCGAGAUUU | |
| | UUUUU | |
| 20 | GUUUUAGUACUCUGUAAUGAAAAUUACAGAAUCUACUAAAACAA | 188 |
| | GGCAAAAUGCCGUGUUUAUCUCGUCAACUUGUUGGCGAGA | |
| 21 | GUUUUAGUACUCUGUAAUGAAAAUUACAGAAUCUACUAAAACAA | 187 |
| | GGCAAAAUGCCGUGUUUAUCUCGUCAACUUGUUGGCGAGAUUU | |
| | UUUUU | |
| 22 | GUUUAAGUACUCUGUGCUGGAAACAGCACAGAAUCUACUUAAAC | 186 |
| | AAGGCAAAAUGCCGUGUUUAUCUCGUCAACUUGUUGGCGAGAU | |
| | UUUUUUU | |
| 23 | GUUUAAGUACUCUGUGCUGGAAACAGCACAGAAUCUACUUAAAC | 185 |
| | AAGGCAAAAUGCCGUGUUUAUCUCGUCAACUUGUUGGCGAGA | |
| 24 | GUUUAAGUACUCUGUGCUGGAAACAGCACAGAAUCUACUUAAAC | 186 |
| | AAGGCAAAAUGCCGUGUUUAUCUCGUCAACUUGUUGGCGAGAU | |
| | UUUUUUU | |
| 25 | GUUUUAGUACUCUGGAAACAGAAUCUACUAAAACAAGGCAAAAU | 189 |
| | GCCGUGUUUAUCUCGUCAACUUGUUGGCGAGAUUUU | |
| 26 | GUUUUAGUACUCUGGAAACAGAAUCUACUAAAACAAGGCAAAAU | 190 |
| | GCCGUGUUUAUCUCGUCAACUUGUUGGCGAGA | |
| 27 | GUUUUAGUACUCUGGAAACAGAAUCUACUAAAACAAGGCAAAAU | 191 |
| | GCCGUGUUUAUCUCGUCAACUUGUUGGCGAGAUUUUUUUU | |
| 28 | GUUUAAGAGCUAUGCUGGAAACAGCAUAGCAAGUUUAAAUAAGG | 192 |
| | CUAGUCCGUUAUCAACUUGAAAAAGUGGCACCGAGUCGGUGCU | |
| | UUUUUU | |
| 29 | GUUUCAGAGCUAUGCUGGAAACAGCAUAGCAAGUUCAAAUAAGG | 299 |
| | CUAGUCCGUUAUCAACUUGAAAAAGUGGCACCGAGUCGGUGCU | |
| | UUUUU | |
| 30 | GUUUGAGAGCUAUGCUGGAAACAGCAUAGCAAGUUCAAAUAAGG | 300 |
| | CUAGUCCGUUAUCAACUUGAAAAAGUGGCACCGAGUCGGUGCU | |
| | UUUUU | |
| 31 | GUUUCAGAGCUACAGCAGAAAUGCUGUAGCAAGUUGAAAU | 301 |
| | AAGGCUAGUCCGUUAUCAACUUGAAAAAGUGGCACCGAGU | |
| | CGGUGCUUUUUU | |

TABLE 4
Exemplary Nme2Cas9 3′ sgRNA sequences

| 3′ sgRNA | | SEQ |
|---|---|---|
| sequence | Sequence | ID NO |
| 1 | GUUGUAGCUCCCUUUCUCAUUUCGGAAACGAAAUGAGAACCGUU | 193 |
| | GCUACAAUAAGGCCGUCUGAAAAGAUGUGCCGCAACGCUCUGCC | |
| | CCUUAAAGCUUCUGCUUUAAGGGGCAUCGUUUAUUUUUU | |
| 2 | GUUGUAGCUCCCUUUCUCAUUUCGGAAACGAAAUGAGAACCGUU | 194 |
| | GCUACAAUAAGGCCGUCUGAAAAGAUGUGCCGCAACGCUCUGCC | |
| | CCUUAAAGCUUCUGCUUUAAGGGGCAUCGUUUAUUUUU | |
| 3 | GUUGUAGCUCCCUUUCUCAUUUCGGAAACGAAAUGAGAACCGUU | 195 |
| | GCUACAAUAAGGCCGUCUGAAAAGAUGUGCCGCAACGCUCUGCC | |
| | CCUUAAAGCUUCUGCUUUAAGGGGCAUCGUUUAUUUU | |
| 4 | GUUGUAGCUCCCUUUCUCAUUUCGGAAACGAAAUGAGAACCGUU | 196 |
| | GCUACAAUAAGGCCGUCUGAAAAGAUGUGCCGCAACGCUCUGCC | |
| | CCUUAAAGCUUCUGCUUUAAGGGGCAUCGUUUAUUU | |
| 5 | GUUGUAGCUCCCUUUCUCAUUUCGGAAACGAAAUGAGAACCGUU | 197 |
| | GCUACAAUAAGGCCGUCUGAAAAGAUGUGCCGCAACGCUCUGCC | |
| | CCUUAAAGCUUCUGCUUUAAGGGGCAUCGUUUAUU | |
| 6 | GUUGUAGCUCCCUUUCUCAUUUCGGAAACGAAAUGAGAACCGUU | 198 |
| | GCUACAAUAAGGCCGUCUGAAAAGAUGUGCCGCAACGCUCUGCC | |
| | CCUUAAAGCUUCUGCUUUAAGGGGCAUCGUUUAU | |
| 7 | GUUGUAGCUCCCUUUCUCAUUUCGGAAACGAAAUGAGAACCGUU | 199 |
| | GCUACAAUAAGGCCGUCUGAAAAGAUGUGCCGCAACGCUCUGCC | |
| | CCUUAAAGCUUCUGCUUUAAGGGGCAUCGUUUA | |
| 8 | GUAGCUCCCUUUCUCAUUUCGGAAACGAAAUGAGAACCGUUGCU | 200 |
| | ACAAUAAGGCCGUCUGAAAAGAUGUGCCGCAACGCUCUGCCCCU | |
| | UAAAGCUUCUGCUUUAAGGGGCAUCGUUUA | |
| 9 | GUAGCUCCCGAAACGUUGCUACAAUAAGGCCGUCUGAAAAGAUG | 201 |
| | UGCCGCAACGCUCUGCCCCUUAAAGCUUCUGCUUUAAGGGGCA | |
| | UCGUUUA | |
| 10 | GUAGCUCCCGAAACGUUGCUACAAUAAGGCCGUCUGAAAAGAUG | 202 |
| | UGCCGCAACGCUCUGCCCCUUUUCUAAGGGGCAUCGUUUA | |
| 11 | GUAGCUCCCGAAACGUUGCUACAAUAAGGCCGUCUGAAAAGAUG | 203 |
| | UGCCGCAACGCUCUGCCCCUUUUCUAAGGGGCAU | |
| 12 | GUAGCUCCCGAAACGUUGCUACAAUAAGGCCGUCUGAAAAGAUG | 204 |
| | UGCCGCAACGCUCUGCUUCUGCAUCGUU | |
| 13 | GUAGCUCCCGAAACGUUGCUACAAUAAGGCCGUCUGAAAAGAUG | 205 |
| | UGCCGCAACGCUCUGCUUCUGCAUCGUUUA | |
| 14 | GUAGCUCCCGAAACGUUGCUACAAUAAGGCCGUCUGAAAAGAUG | 206 |
| | UGCCGCAACGCUCUGCCCUUCUGGGCAUCGUU | |
| 15 | GUAGCUCCCGAAACGUUGCUACAAUAAGGCCGUCUGAAAAGAUG | 207 |
| | UGCCGCAACGCUCUGCCCCUUUCUAGGGGCAUCGUU | |
| 16 | GUAGCUCCCGAAACGUUGCUACAAUAAGGCCGUCUGAAAAGAUG | 208 |
| | UGCCGCAACGCUCUGCCCCUUCUGGGGCAUCGUU | |
| 17 | GUAGCUCCCGAAACGUUGCUACAAUAAGGCCGUCUGAAAAGAUG | 206 |
| | UGCCGCAACGCUCUGCCCUUCUGGGCAUCGUU | |
| 18 | GUAGCUCCCGAAACGUUGCUACAAUAAGGCCGUCUGAAAAGAUG | 209 |
| | UGCCGCAACGCUCUGCCUUCUGGCAUCGUU | |
| 19 | GUAGCUCCCGAAACGUUGCUACAAUAAGGCCGUCUGAAAAGAUG | 209 |
| | UGCCGCAACGCUCUGCCUUCUGGCAUCGUU | |
| 20 | GUUGUAGCUCCCUUUCUCGAAAGAGAACCGUUGCUACAAUAAGG | 210 |
| | CCGUCUGAAAAGAUGUGCCGCAACGCUCUGCCCCUUAAAGCUUC | |
| | UGCUUUAAGGGGCAUCGUUUA | |
| 21 | GUUGUAGCUCCCGAAACGUUGCUACAAUAAGGCCGUCUGAAAAG | 211 |
| | AUGUGCCGCAACGCUCUGCCCCUUAAAGCUUCUGCUUUAAGGG | |
| | GCAUCGUUUA | |
| 22 | GUUGUAGCUCCCGAAACGUUGCUACAAUAAGGCCGUCUGAAAAG | 212 |
| | AUGUGCCGCAACGCUCUGCCUUCUGGCAUCGUU | |

The sgRNA can comprise no uracil base at the 3′ end of the sgRNA sequence. The sgRNA can comprise one or more uracil bases at the 3′ end of the sgRNA sequence. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence. Different length stretches of uracil can be appended at the 3′end of a sgRNA as terminators. Thus, for example, the 3′ sgRNA sequences set forth in Table 3 and Table 4 can be modified by adding or removing one or more uracils at the end of the sequence.

6.2.3. Modified gRNA molecules

Guide RNAs can be readily synthesized by chemical means, enabling a number of modifications to be readily incorporated, as described in the art. The disclosed gRNA (e.g., sgRNA) molecules can be unmodified or can contain any one or more of an array of chemical modifications.

While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach that can be used for generating chemically modified RNAs of greater length is to produce two or more molecules that are ligated together. Much longer RNAs, such as those encoding a Cas9 endonuclease, are more readily generated enzymatically. While fewer types of modifications are available for use in enzymatically produced RNAs, there are still modifications that can be used to, for instance, enhance stability, reduce the likelihood or degree of innate immune response, and/or enhance other attributes, as described herein and in the art.

By way of illustration of various types of modifications, especially those used frequently with smaller chemically synthesized RNAs, modifications can comprise one or more nucleotides modified at the 2′ position of the sugar, for instance a 2′-O-alkyl, 2′-O-alkyl-O-alkyl, or 2′-fluoro-modified nucleotide. In some examples, RNA modifications can comprise 2′-fluoro, 2′-amino or 2′-O-methyl modifications on the ribose of pyrimidines, abasic residues, or an inverted base at the 3′ end of the RNA. Such modifications can be routinely incorporated into oligonucleotides and these oligonucleotides have been shown to have a higher Tm (thus, higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, —N(CH₃)—O—CH₂ (known as a methylene(methylimino) or MMI backbone), CH₂—O—N(CH₃)—CH₂, CH₂—N (CH₃)—N(CH₃)—CH₂ and O—N(CH₃)—CH₂—CH₂ backbones, wherein the native phosphodiester backbone is represented as O- P- O- CH,); amide backbones (see De Mesmaeker et al. 1995, Ace. Chem. Res., 28:366-374); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., 1991, Science 254:1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, 2002, Biochemistry, 41(14):4503-4510; Genesis, Volume 30, Issue 3, (2001); Heasman, 2002, Dev. Biol., 243: 209-214; Nasevicius et al., 2000, Nat. Genet., 26:216-220; Lacerra et al., 2000, Proc. Natl. Acad. Sci., 97: 9591-9596; and U.S. Pat. No. 5,034,506.

Cyclohexenyl nucleic acid oligonucleotide mimetics are described in Wang et al., 2000, J. Am. Chem. Soc., 122: 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic intemucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

One or more substituted sugar moieties can also be included, e.g., one of the following at the 2′ position: OH, SH, SCH₃, F, OCN, OCH₃, OCH₃O(CH₂)n CHs, O(CH₂)n NH₂, or O(CH₂)n CHs, where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or bi-alkyl; O-, S-, or N-alkenyl; SOCH₃; SO₂CH₃; ONO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some aspects, a modification includes 2′-methoxyethoxy (2′-O—CH₂CH₂OCH₃, also known as 2′-O-(2-methoxyethyl)) (Martin et al., 1995, Helv. Chim. Acta, 78, 486). Other modifications include 2′-methoxy (2′-O—CH₃), 2′-propoxy (2′-OCH₂CH₂CH₃) and 2′-fluoro (2′-F). Similar modifications can also be made at other positions on the oligonucleotide, particularly the 3′ position of the sugar on the 3′ terminal nucleotide and the 5′ position of 5′ terminal nucleotide. Oligonucleotides can also have sugar mimetics, such as cyclobutyls in place of the pentofuranosyl group.

In some examples, both a sugar and an internucleoside linkage (in the backbone) of the nucleotide units can be replaced with novel groups. The base units can be maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide can be replaced with an amide containing backbone, for example, an aminoethylglycine backbone. The nucleobases can be retained and bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al., 1991, Science, 254: 1497-1500.

RNAs such as guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5- methylcytosine (also referred to as 5-methyl-2′ deoxy cytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl)adenine, 2-(aminoalklyamino) adenine or other heterosub stituted alkyladenines, 2-thiouracil, 2-thiothymine, 5-bromouracil, 5-hydroxymethyluracil, 8-azaguanine, 7- deazaguanine, N6 (6-aminohexyl) adenine, and 2,6-diaminopurine. Komberg, A., DNA Replication, W. H. Freeman & Co., San Francisco, pp. 75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by about 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Further, nucleobases can comprise those disclosed in U.S. Pat. No. 3,687,808, those disclosed in ‘The Concise Encyclopedia of Polymer Science and Engineering’, 858-859, Kroschwitz, J. I., ed. John Wiley & Sons, 1990, those disclosed by Englisch et al., Angewandle Chemie, International Edition’, 1991, 30, p. 613, and those disclosed by Sanghvi, Y. S., Chapter 15, Antisense Research and Applications’, 289-302, Crooke, S. T. and Lebleu, B. ea., CRC Press, 1993. Certain of these nucleobases can be useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by about 0.6-1.2° C. (Sanghvi, Y. S., Crooke, S. T. and Lebleu, B., eds, ‘Antisense Research and Applications’, CRC Press, Boca Raton, 1993, 276-278) and are aspects of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and U.S. Patent Application Publication 2003/0158403.

Thus, a modified gRNA can include, for example, one or more non-natural sugars, internucleotide linkages and/or bases. It is not necessary for all positions in a given gRNA to be uniformly modified, and in fact more than one of the aforementioned modifications can be incorporated in a single oligonucleotide, or even in a single nucleoside within an oligonucleotide.

The guide RNAs and/or mRNA (or DNA) encoding an endonuclease can be chemically linked to one or more moieties or conjugates that enhance the activity, cellular distribution, or cellular uptake of the oligonucleotide. Such moieties comprise, but are not limited to, lipid moieties such as a cholesterol moiety (Letsinger et al. 1989, Proc. Natl. Acad. Sci. USA, 86: 6553-6556); cholic acid (Manoharan et al, 1994, Bioorg. Med. Chem. Let., 4: 1053-1060); a thioether, e.g., hexyl-S-tritylthiol (Manoharan et al, 1992, Ann. N. Y. Acad. Sci., 660: 306-309; Manoharan et al., 1993, Bioorg. Med. Chem. Let., 3: 2765-2770); a thiocholesterol (Oberhauser et al., 1992, Nucl. Acids Res., 20: 533-538); an aliphatic chain, e.g., dodecandiol or undecyl residues (Kabanov et al, 1990, FEBS Lett., 259: 327-330; Svinarchuk et al, 1993, Biochimie, 75: 49-54); a phospholipid, e.g., di-hexadecyl-rac-glycerol or triethylammonium 1,2-di-O-hexadecyl-rac-glycero-3-H-phosphonate (Manoharan et al., 1995, Tetrahedron Lett., 36: 3651-3654; and Shea et al, 1990, Nucl. Acids Res., 18: 3777-3783); a polyamine or a polyethylene glycol chain (Mancharan et al, 1995, Nucleosides & Nucleotides, 14: 969-973); adamantane acetic acid (Manoharan et al, 1995, Tetrahedron Lett., 36: 3651-3654); a palmityl moiety (Mishra et al., 1995, Biochim. Biophys. Acta, 1264: 229-237); or an octadecylamine or hexylamino-carbonyl-t oxycholesterol moiety (Crooke et al, 1996, J. Pharmacol. Exp. Ther., 277: 923-937). See also U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203; 5,451,463; 5,510,475; 5,512,667; 5,514,785; 5,565,552; 5,567,810; 5,574,142; 5,585,481; 5,587,371; 5,595,726; 5,597,696; 5,599,923; 5,599, 928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); see, e.g., Hu, et al., 2014, Protein Pept Lett. 21(10):1025-30. Other systems known in the art and regularly developed can be used to target biomolecules of use in the present case and/or complexes thereof to particular target cells of interest.

Targeting moieties or conjugates can include conjugate groups covalently bound to functional groups, such as primary or secondary hydroxyl groups. Conjugate groups of the present disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this present disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compounds of the present disclosure. Representative conjugate groups are disclosed in International Patent Application Publication WO1993007883, and U.S. Pat. No. 6,287,860. Conjugate moieties include, but are not limited to, lipid moieties such as a cholesterol moiety, cholic acid, a thioether, e.g., hexyl-5-trityl thiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

A large variety of modifications have been developed and applied to enhance RNA stability, reduce innate immune responses, and/or achieve other benefits that can be useful in connection with the introduction of polynucleotides into human cells, as described herein; see, e.g., the reviews by Whitehead K A et al., 2011, Annual Review of Chemical and Biomolecular Engineering, 2: 77-96; Gaglione and Messere, 2010, Mini Rev Med Chem, 10(7):578-95; Chernolovskaya et al, 2010, Curr Opin Mol Ther., 12(2): 158-67; Deleavey et al., 2009, Curr Protoc Nucleic Acid Chem Chapter 16:Unit 16.3; Behlke, 2008, Oligonucleotides 18(4):305-19; Fucini et al, 2012, Nucleic Acid Ther 22(3): 205-210; Bremsen et al, 2012, Front Genet 3: 154.

### 6.3. DNA Endonucleases

The gRNAs of the disclosure can be used to direct a DNA endonuclease to a RHO gene having a P347 mutation. The DNA endonuclease can be, for example, a Type II CRISPR endonuclease such as Cas9. A DNA endonuclease can be provided to a cell or a subject as one or more polypeptides, or one or more nucleic acids (e.g., mRNAs) encoding the one or more polypeptides.

The DNA endonuclease can be, for example, a wild-type Cas9 protein or a Cas9 variant having one or more mutations relative to a wild-type Cas9 protein. For example, the Cas9 (or Cas9 variant) can be S. pyogenes Cas9 (“SpCas9”) or an SpCas9 orthologue such as Cas9 from S. thermophilus, S. aureus or N meningitides. In some embodiments, the Cas9 is a Nme2Cas9. Exemplary nucleic acid and amino acid sequences for Nme2Cas9 proteins are described in US 2019/0338308, the contents of which are incorporated herein by reference in their entirety.

In some embodiments, the DNA endonuclease used in the compositions and methods of the disclosure is a Cas9 variant, for example, a SpCas9 variant. For example, Cas9 variants described in WO 2018/149888, the contents of which are incorporated herein by reference in their entireties, can be used. As another example, enzymes or orthologs listed as SEQ ID NOs. 1-612 of WO 2019/102381, the contents of which are incorporated herein by reference in their entireties, and variants thereof, can be utilized in the compositions and methods described herein.

In some embodiments, the Cas9 variant, when used with a gRNA of the disclosure, preferentially cleaves a RHO gene having a P347 mutation over a RHO gene not having a P347 mutation. The degree of preference can be quantitated, for example, by measuring the percentage editing of a RHO gene having a P347 mutation and the percentage editing of a RHO gene not having a P347 mutation in a population of cells (e.g., HEK293 cells) each containing a RHO gene having a P347 mutation and a RHO gene not having a P347 mutation. In some embodiments, the preferential cleavage of the RHO gene having a P347 mutation over cleavage of the RHO gene not having a P347 mutation is by a factor of at least 1.3, at least 1.5, at least 2, at least 2.5, at least 3, at least 4, at least 5, at least 10, at least 11, or at least 100. In some embodiments, the preferential cleavage of the RHO gene having a P347 mutation over cleavage of the RHO gene not having a P347 mutation can be by a factor of 1.3 to 100, 2 to 100, 5 to 100, 10 to 100, 1.3-11, 2-11, 2.5-11, 3-11, 4-11, 5-11, 1.1-5, 1.3-5, 2-10, 3-10, 4-10, 5-10, 2-4, 2-5, 2-4, 3-5, or 4-5.

The DNA endonuclease can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary DNA endonuclease, e.g., Cas9 from S. pyogenes having the reference sequence of NP_269215 (NCBi):

(SEQ ID NO: 1)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

In other embodiments, the DNA endonuclease can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type Nme2Cas9 protein having the sequence:

(SEQ ID NO: 2)
MAAFKPNPINYILGLDIGIASVGWAMVEIDEEENPIRLIDLGVRVFERAE
VPKTGDSLAMARRLARSVRRLTRRRAHRLLRARRLLKREGVLQAADFDEN
GLIKSLPNTPWQLRAAALDRKLTPLEWSAVLLHLIKHRGYLSQRKNEGET
ADKELGALLKGVANNAHALQTGDFRTPAELALNKFEKESGHIRNQRGDYS
HTFSRKDLQAELILLFEKQKEFGNPHVSGGLKEGIETLLMTQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSSELQDEIGTAFSLFKTDEDITGRLK
DRVQPEILEALLKHISFDKFVQISLKALRRIVPLMEQGKRYDEACAEIYG
DHYGKKNTEEKIYLPPIPADEIRNPVVLRALSQARKVINGVVRRYGSPAR
IHIETAREVGKSFKDRKEIEKRQEENRKDREKAAAKFREYFPNFVGEPKS
KDILKLRLYEQQHGKCLYSGKEINLVRLNEKGYVEIDHALPFSRTWDDSF
NNKVLVLGSENQNKGNQTPYEYFNGKDNSREWQEFKARVETSRFPRSKKQ
RILLQKFDEDGFKECNLNDTRYVNRFLCQFVADHILLTGKGKRRVFASNG
QITNLLRGFWGLRKVRAENDRHHALDAVVVACSTVAMQQKITRFVRYKEM
NAFDGKTIDKETGKVLHQKTHFPQPWEFFAQEVMIRVFGKPDGKPEFEEA
DTPEKLRTLLAEKLSSRPEAVHEYVTPLFVSRAPNRKMSGAHKDTLRSAK
RFVKHNEKISVKRVWLTEIKLADLENMVNYKNGREIELYEALKARLEAYG
GNAKQAFDPKDNPFYKKGGQLVKAVRVEKTQESGVLLNKKNAYTIADNGD
MVRVDVFCKVDKKGKNQYFIVPIYAWQVAENILPDIDCKGYRIDDSYTFC
FSLHKYDLIAFQKDEKSKVEFAYYINCDSSNGRFYLAWHDKGSKEQQFRI
STQNLVLIQKYQVNELGKEIRPCRLKKRPPVR.

In other embodiments, the DNA endonuclease can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the following Nme2Cas9 protein sequence, which includes a tag and a nuclear localization signal:

(SEQ ID NO: 213)
MVPKKKRKVEDKRPAATKKAGQAKKKKMAAFKPNPINYILGLDIGIASVG
WAMVEIDEEENPIRLIDLGVRVFERAEVPKTGDSLAMARRLARSVRRLTR
RRAHRLLRARRLLKREGVLQAADFDENGLIKSLPNTPWQLRAAALDRKLT
PLEWSAVLLHLIKHRGYLSQRKNEGETADKELGALLKGVANNAHALQTGD
FRTPAELALNKFEKESGHIRNQRGDYSHTFSRKDLQAELILLFEKQKEFG
NPHVSGGLKEGIETLLMTQRPALSGDAVQKMLGHCTFEPAEPKAAKNTYT
AERFIWLTKLNNLRILEQGSERPLTDTERATLMDEPYRKSKLTYAQARKL
LGLEDTAFFKGLRYGKDNAEASTLMEMKAYHAISRALEKEGLKDKKSPLN
LSSELQDEIGTAFSLFKTDEDITGRLKDRVQPEILEALLKHISFDKFVQI
SLKALRRIVPLMEQGKRYDEACAEIYGDHYGKKNTEEKIYLPPIPADEIR
NPVVLRALSQARKVINGVVRRYGSPARIHIETAREVGKSFKDRKEIEKRQ
EENRKDREKAAAKFREYFPNFVGEPKSKDILKLRLYEQQHGKCLYSGKEI
NLVRLNEKGYVEIDHALPFSRTWDDSFNNKVLVLGSENQNKGNQTPYEYF
NGKDNSREWQEFKARVETSRFPRSKKQRILLQKFDEDGFKECNLNDTRYV
NRFLCQFVADHILLTGKGKRRVFASNGQITNLLRGFWGLRKVRAENDRHH
ALDAVVVACSTVAMQQKITRFVRYKEMNAFDGKTIDKETGKVLHQKTHFP
QPWEFFAQEVMIRVFGKPDGKPEFEEADTPEKLRTLLAEKLSSRPEAVHE
YVTPLFVSRAPNRKMSGAHKDTLRSAKRFVKHNEKISVKRVWLTEIKLAD
LENMVNYKNGREIELYEALKARLEAYGGNAKQAFDPKDNPFYKKGGQLVK
AVRVEKTQESGVLLNKKNAYTIADNGDMVRVDVFCKVDKKGKNQYFIVPI
YAWQVAENILPDIDCKGYRIDDSYTFCFSLHKYDLIAFQKDEKSKVEFAY
YINCDSSNGRFYLAWHDKGSKEQQFRISTQNLVLIQKYQVNELGKEIRPC
RLKKRPPVREDKRPAATKKAGQAKKKKYPYDVPDYAGYPYDVPDYAGSYP
YDVPDYAAAPAAKKKKLD.

The DNA endonuclease can comprise at least 70, 75, 80, 85, 90, 95, 97, 99, or 100% identity to a wild-type DNA endonuclease (e.g., Cas9 from S. pyogenes or a Nme2Cas9, supra) over 10 contiguous amino acids. The DNA endonuclease can comprise, in some embodiments, at most: 70, 75, 80, 85, 90, 95, 97, 99, or 100% identity to a wild-type Cas9 (e.g., Cas9 from S. pyogenes or a Nme2Cas9, supra) over 10 contiguous amino acids. The DNA endonuclease can comprise, in some embodiments, at least: 70, 75, 80, 85, 90, 95, 97, 99, or 100% identity to a wild-type DNA endonuclease (e.g., Cas9 from S. pyogenes or a Nme2Cas9, supra) over 10 contiguous amino acids in a HNH nuclease domain of the DNA endonuclease. The DNA endonuclease can comprise, in some embodiments, at most: 70, 75, 80, 85, 90, 95, 97, 99, or 100% identity to a wild-type DNA endonuclease (e.g., Cas9 from S. pyogenes or a Nme2Cas9, supra) over 10 contiguous amino acids in a HNH nuclease domain of the DNA endonuclease. The DNA endonuclease can comprise, in some embodiments, at least: 70, 75, 80, 85, 90, 95, 97, 99, or 100% identity to a wild-type DNA endonuclease (e.g., Cas9 from S. pyogenes or a Nme2Cas9, supra) over 10 contiguous amino acids in a RuvC nuclease domain of the DNA endonuclease. The DNA endonuclease can, in some embodiments, comprise at most: 70, 75, 80, 85, 90, 95, 97, 99, or 100% identity to a wild-type DNA endonuclease (e.g., Cas9 from S. pyogenes or a Nme2Cas9, supra) over 10 contiguous amino acids in a RuvC nuclease domain of the DNA endonuclease.

In some embodiments, the DNA endonuclease used is a Cas9 comprising at least one mutation located at an amino acid residue positions selected in the group consisting of: K377, E387, D397, R400, D406, A421, L423, R424, Q426, Y430, K442, P449, V452, A456, R457, W464, M465, K468, E470, T474, P475, W476, F478, K484, S487, A488, T496, F498, L502, N504, K506, P509, F518, N522, E523, K526, L540, S541, 1548, D550, F553, V561, K562, E573, A589, L598, D605, L607, N609, N612, E617, D618, D628, R629, R635, K637, L651, K652, R654, T657, G658, L666, K673, S675C, I679V, L680, L683, N690, R691, N692, F693, S701, F704, Q712, G715, Q716, H723, I724, L727, I733, L738 and Q739. wherein the position of the modified amino acids sequence is identified by reference to the amino acid numbering in the corresponding position of an unmodified mature SpCas9, as identified by SEQ ID NO:1.

In a preferred embodiment, the modified Cas9 comprises at least one mutation at position K526. Preferably, the mutation at position K526 is K526N or K526E. In some embodiments, the mutation is K526E. In some embodiments, the mutation is K526N.

A variant Cas9 having K526 mutated can comprise one or more further mutations (e.g., 1-9, 1-8, 1-5, 1-4, 4-8, 2-6, 1, 2, 3, 4, 5, 6, 7, or 8), for example located at one or more of the following amino acid residue positions:

K377, E387, D397, R400, Q402, R403, F405, D406, N407, A421, L423, R424, Q426, Y430, K442, P449, Y450, V452, A456, R457, W464, M465, K468, E470, T472, I473, T474, P475, W476, F478, K484, S487, A488, M495, T496, N497, F498, L502, N504, K506, P509, Y515, F518, N522, E523, L540, S541, I548, D550, F553, V561, K562, E573, A589, L598, D605, L607, N609, N612, E617, D618, D628, R629, R635, K637, L651, K652, R654, T657, G658, W659, R661, L666, K673, S675, I679, L680, L683, N690, R691, N692, F693, Q695, H698, S701, F704, Q712, G715, Q716, H721, H723, 1724, L727, A728, 1733, L738, Q739.

In some embodiments, the one or more further mutations comprise one or more of the following:

K377E, E387V, D397E, R400H, Q402R, R403H, F405L, D406Y, D406V, N407P, N407H, A421V, L423P, R424G, Q426R, Y430C, K442N, P449S, Y450A, Y450S, Y450H, Y450N, V452I, A456T, R457P, R457Q, W464L, M465R, K468N, E470D, T472A, I473F, I473V, T474A, P475H, W476R, F478Y, F478V, K484M, S487Y, A488V, M495V, M495T, T496A, N497A, F498I, F498Y, L502P, N504S, K506N, P509L, Y515N, F518L, F518I, N522K, N522I, E523K, E523D, L540Q, S541P, I548V, D550N, F553L, V561M, V561A, K562E, E573D, A589T, L598P, D605V, L607P, N609D, N609S, N612Y, N612K, E617K, D618N, D628G, R629G, R635G, K637N, L651P, L651H, K652E, R654H, T657A, G658E, W659R, R661A, R661W, R661L, R661Q, R661S, L666P, K673M, S675C, I679V, L680P, L683P, N690I, R691Q, R691L, N692I, F693Y, Q695A, Q695H, Q695L, H698Q, H698P, S701F, F704S, Q712R, G715S, Q716H, H721R, H723L, I724V, L727H, A728G, A728T, I733V, L738P, Q739E, Q739P, Q739K.

In some embodiments, the Cas9 variant has an amino acid sequence with at least 50%, 60%, 70%, 80%, 90%, 95%, 99% or 100% identity with SEQ ID NO:1.

In some embodiments, the Cas9 variant comprises a mutation at position K526 and one or more further mutations at one or more of positions Y450, M495, Y515, R661, N690, R691, Q695, H698; preferably M495, Y515, R661, and H698, for example, Y450S, M495V, Y515N, R661X, N690I, R691Q, Q695H, H698Q, preferably selected from M495V, Y515N, K526E, R661X, H698Q, where X is L, Q or S, preferably where X is Q or S.

In some embodiments, a Cas9 variant comprises a double mutation selected from K526E+Y450S, K526E+M495V, K526E+Y515N, K526E+R661X, K526E+N690I, K526E+R691Q, K526E+Q695H and K526E+H698Q; wherein X is L, Q or S, preferably where X is Q or S.

In some embodiments, a Cas9 variant comprises a triple mutation selected from M495V+K526E+R661X, Y515N+K526E+R661X, K526E+R661X+H698Q and M495V+Y515N+K526E, where X is L, Q or S, preferably where X is Q or S.

In some embodiments, a Cas9 variant comprises a quadruple mutation selected from M495V+Y515N+K526E+R661X and M495V+K526E+R661X+H698Q, where X is L, Q or S, preferably where X is Q or S.

In some embodiments, the Cas9 variant comprises the mutations M495V+Y515N+K526E+R661Q (hereinafter also named evoCas9). In other embodiments, the Cas9 variant comprises the mutations M495V+Y515N+K526E+R661S (hereinafter named evoCas9-II).

In some embodiments, a Cas9 variant comprises at least one of the following mutations:

K377E, E387V, D397E, R400H, Q402R, R403H, F405L, D406Y, D406V, N407P, N407H, A421V, L423P, R424G, Q426R, Y430C, K442N, P449S, Y450S, Y450H, Y450N, V452I, A456T, R457P, R457Q, W464L, M465R, K468N, E470D, T472A, I473F, I473V, T474A, P475H, W476R, F478Y, F478V, K484M, S487Y, A488V, M495V, M495T, T496A, F498I, F498Y, L502P, N504S, K506N, P509L, Y515N, F518L, F518I, N522K, N522I, E523K, E523D, K526E, K526N, L540Q, S541P, I548V, D550N, F553L, V561M, V561A, K562E, E573D, A589T, L598P, D605V, L607P, N609D, N609S, N612Y, N612K, E617K, D618N, D628G, R629G, R635G, K637N, L651P, L651H, K652E, R654H, T657A, G658E, W659R, R661W, R661L, R661Q, R661S, L666P, K673M, S675C, I679V, L680P, L683P, N690I, R691Q, R691L, N692I, F693Y, Q695H, Q695L, H698Q, H698P, S701F, F704S, Q712R, G715S, Q716H, H721R, H723L, I724V, L727H, A728G, A728T, I733V, L738P, Q739E, Q739P, Q739K.

In some embodiments, a Cas9 variant comprises a single mutation selected from D406Y, W464L, T474A, K526E, N612K, and L683P.

In some embodiments, a Cas9 variant comprises a double mutation selected from R400H+Y450S, D406V+E523K, A421V+R661W, R424G+Q739P, W476R+L738P, P449S+F704S, N522K+G658E, E523D+E617K, L540Q+L607P, W659R+R661W, S675C+Q695L and I679V+H723L.

In some embodiments, a Cas9 variant comprises three mutations selected from K377E+L598P+L651H, D397E+Y430C+L666P, Q402R+V561M+Q695L, N407P+F498I+P509L, N407H+K637N+N690I, Y450H+F553L+Q716H, Y450N+H698P+Q739K, T472A+P475H+A488V, I473F+D550N+Q739E, F478Y+N522I+L727H, K484M+Q695H+Q712R, S487Y+N504S+E573D, T496A+N609D+A728G, R654H+R691Q+H698Q and R691L+H721R+I733V.

In some embodiments, a Cas9 variant comprises four mutations selected from F405L+F518L+L651P+I724V, L423P+M465R+Y515N+K673M, R457P+K468N+R661W+G715S, E470D+I548V+A589T+Q695H, A488V+D605V+R629G+T657A and M495V+K526N+S541P+K562E.

In some embodiments, a Cas9 variant comprises the five mutations R403H+N612Y+L651P+K652E+G715S.

In some embodiments, a Cas9 variant comprises six mutations from E387V+V561A+D618N+D628G+L680P+S701F, R403H+K526E+N612Y+L651P+K652E+G715S, R403H+M495T+N612Y+L651P+K652E+G715S, R403H+L502P+N612Y+L651P+K652E+G715S, R403H+K506N+N612Y+L651P+K652E+G715S, and R403H+N612Y+L651P+K652E+N692I+G715S.

In some embodiments, a Cas9 variant comprises seven mutations selected from R403H+A456T+N612Y+L651P+K652E+G715S+G728T, R403H+F498Y+N612Y+L651P+K652E+R661L+G715S, and R403H+Q426R+F478V+N612Y+L651P+K652E+G715S.

In some embodiments, a Cas9 variant comprises the following eight mutations R403H+R442N+V452I+N609S+N612Y+R635G+L651P+K652E+F693Y+G715S.

In some embodiments, a Cas9 variant comprises the following nine mutations R403H+R457Q+F518I+N612Y+R635G+L651P+K652E+F693Y+G715S.

In some embodiments a Cas9 variant comprises at least one mutation selected from Y450S, M495V, Y515N, K526E, R661X, N690I, R691Q, Q695H, and H698Q, where X is L, Q or S, preferably where X is Q or S.

In some embodiments, a Cas9 variant comprises N692A, M694A, Q695A, and H698A mutations (see Ikeda et al., 2019, Commun Biol 2,371, describing a Cas9 variant with these mutations identified as HypaCas9)

In some embodiments, a Cas9 variant comprises K848A, K1003A, and R1060A mutations (see Slaymaker et al., 2016, Science, 351(6268):84-88, describing a Cas9 variant with these mutations identified as eSpCas9(1.1)).

In some embodiments, a Cas9 variant comprises F539S, M763I, and K890N mutations (see Lee et al., 2018, Nat Commun. 9:3048, describing a Cas9 variant with these mutations identified as Sniper-Cas).

In some embodiments, a Cas9 variant comprises N497A, R661A, Q695A, and Q926A mutations (see Kleinstiver et al. 2016, Nature, 529:490-495, describing a Cas9 variant with these mutations identified as SpCas9-HF1).

In some embodiments, a Cas9 variant comprises a R691A mutation (see Vakulskas et al., 2018, Nat Med 24:1216-1224, describing a Cas9 variant with these mutations identified as HiFi Cas9).

Cas9 variants described herein can further comprise one or more additional mutations, for example at residues L169A, K810A, K848A, Q926A, R1003A, R1060A, and D1135E.

Cas9 variants having mutations described above (e.g., evoCas9, HypaCas9, eSpCas9(1.1), Sniper-Cas, SpCas9-HF1, HiFi Cas9) can have improved specificity compared to wild-type Cas9 and other reported Cas9 variants. In some embodiments, the mutations identified above for Cas9 are suitable to improve the specificity of other Cas9 nickase, dCas9-Fokl or dCas9. Therefore, optionally the above described Cas9 variants can further comprise at least one additional mutation at a residue selected in the group consisting of D10, E762, D839, H840, N863, H983 and D986 to decrease nuclease activity. In some embodiments, such additional mutations are D10A, or D10N and H840A, H840N or H840Y. In some embodiments, said mutations result in a Cas9 nickase or in a catalytically inactive Cas9 (Ran F et al., 2013, Cell, 154(6):1380-1389; Maeder M et al., Nature Methods., 2013, 10(10):977-979).

In some embodiments, a Cas9 variant can have improved the specificity for recognizing alternative PAM sequences. Therefore, optionally the above described Cas9 variants can further comprise one or more additional mutations at residues D1135V/R1335Q/T1337R (QVR variant), D1135E/R1335Q/T1337R (EVR variant), D1135V/G1218R/R1335Q/T1337R (VRQR variant), D1135V/G1218R/R1335E/T1337R (VRER variant), as described in US US2016/0319260, the contents of which are incorporated by reference in their entirety.

A modified form of a DNA endonuclease such as a Cas9 protein can comprise a mutation such that it can induce a SSB on a target nucleic acid (e.g., by cutting only one of the sugar-phosphate backbones of a double-strand target nucleic acid). The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes, supra). The mutation can result in one or more of the plurality of nucleic acid-cleaving domains retaining the ability to cleave the complementary strand of the target nucleic acid, but reducing its ability to cleave the non-complementary strand of the target nucleic acid. The mutation can result in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp 10, His840, Asn854 and Asn856, can be mutated to inactivate one or more of the plurality of nucleic acid-cleaving domains (e.g., nuclease domains). The residues to be mutated can correspond to residues Asp 10, His840, Asn854 and Asn856 in the wild-type exemplary S. pyogenes Cas9 polypeptide (e.g., as determined by sequence and/or structural alignment). Non-limiting examples of mutations include D10A, H840A, N854A or N856A. Mutations other than alanine substitutions can be suitable.

A D10A mutation can be combined with one or more of H840A, N854A, or N856A mutations to produce a DNA endonuclease substantially lacking DNA cleavage activity. A H840A mutation can be combined with one or more of D10A, N854A, or N856A mutations to produce a DNA endonuclease substantially lacking DNA cleavage activity. A N854A mutation can be combined with one or more of H840A, D10A, or N856A mutations to produce a DNA endonuclease substantially lacking DNA cleavage activity. A N856A mutation can be combined with one or more of H840A, N854A, or D10A mutations to produce a DNA endonuclease substantially lacking DNA cleavage activity. DNA endonucleases that comprise one substantially inactive nuclease domain are referred to as “nickases”.

Nickase variants of RNA-guided endonucleases, for example Cas9, can be used to increase the specificity of CRISPR-mediated genome editing. Wild type Cas9 is typically guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes.

The DNA endonuclease can comprise an amino acid sequence comprising at least 15% amino acid identity (e.g., 25% or more, 50% or more, 75% or more, 85% or more, 90% or more, 95% or more) to a Cas9 from a bacterium (e.g, S. pyogenes), a nucleic acid binding domain, and two nucleic acid cleaving domains (e.g., a HNH domain and a RuvC domain).

The DNA endonuclease can comprise an amino acid sequence comprising at least 15% amino acid identity (e.g., 25% or more, 50% or more, 75% or more, 85% or more, 90% or more, 95% or more) to a Cas9 from a bacterium (e.g, S. pyogenes), and two nucleic acid cleaving domains, wherein one or both of the nucleic acid cleaving domains comprise at least 50% amino acid identity to a nuclease domain from Cas9 from a bacterium (e.g., S. pyogenes).

The DNA endonuclease can comprise an amino acid sequence comprising at least 15% amino acid identity (e.g., 25% or more, 50% or more, 75% or more, 85% or more, 90% or more, 95% or more) to a Cas9 from a bacterium (e.g, S. pyogenes), two nucleic acid cleaving domains (e.g., a HNH domain and a RuvC domain), and non-native sequence (for example, a nuclear localization signal) or a linker linking the DNA endonuclease to a non native sequence.

The DNA endonuclease can comprise an amino acid sequence comprising at least 15% amino acid identity (e.g., 25% or more, 50% or more, 75% or more, 85% or more, 90% or more, 95% or more) to a Cas9 from a bacterium (e.g., S. pyogenes), two nucleic acid cleaving domains (e.g., a HNH domain and a RuvC domain), wherein the DNA endonuclease comprises a mutation in one or both of the nucleic acid cleaving domains that reduces the cleaving activity of the nuclease domains by at least 50%.

The DNA endonuclease can comprise an amino acid sequence comprising at least 15% amino acid identity (e.g., 25% or more, 50% or more, 75% or more, 85% or more, 90% or more, 95% or more) to a Cas9 from a bacterium (e.g., S. pyogenes), and two nucleic acid cleaving domains (e.g., a HNH domain and a RuvC domain), wherein one of the nuclease domains comprises mutation of aspartic acid 10, and/or wherein one of the nuclease domains can comprise a mutation of histidine 840, and wherein the mutation reduces the cleaving activity of the nuclease domain(s) by at least 50%.

One or more DNA endonucleases, e.g., as described herein, can be used together, to effect one double-strand break at a specific locus in the genome, or in another embodiment, four nickases that together effect or cause two double-strand breaks at specific loci in the genome can be used. In other embodiments, one DNA endonuclease which can effect or cause one double-strand break at a specific locus in the genome is used.

Non-limiting examples of Cas9 orthologs from other bacterial strains that can be used include, but are not limited to, Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca Arthrospira sp. PCC 8005; Bacillus pseudomycoides DSM 12442; Bacillus selenitireducens MLS10; Burkholderiales bacterium 1 1 47; Caldicelulosiruptor becscii DSM 6725; Candidatus Desulforudis audaxviator MP104C; Caldicellulosiruptor hydrolhermahs 108; Clostridium phage c-st; Clostridium botulinum A3 str. Loch Maree Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMMO 1; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum SI; Petrotoga mobilis SJ95; Polaromonas naphthalenivorans CJ2; Polaromonas sp. JS666; Pseudoalteromonas haloplanktis TAC125; Streptomyce pristinae spiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10(5): 726-737.

In addition to Cas9 orthologs, other Cas9 variants such as fusion proteins of inactive dCas9 and effector domains with different functions can be served as a platform for genetic modulation. Any of the foregoing enzymes can be useful in the present disclosure.

Further examples of endonucleases that can be utilized in the present disclosure are provided in SEQ ID NOs: 1-612 of WO 2019/102381. These proteins, and any other described herein, can be modified before use or can be encoded in a nucleic acid sequence such as a DNA, RNA or mRNA or within a vector construct such as the plasmids or adeno-associated virus (AAV) vectors described herein. Further, they can be codon optimized.

In some embodiments, the disclosed endonuclease can contain artificial, synthetic, or non-classical amino acids or chemical amino acid analogs. Non-classical amino acids include, but are not limited to, the D-isomers of the common amino acids, fluoro-amino acids, and “designer” amino acids such as 3-methyl amino acids, Cy-methyl amino acids, Ny-methylamino acids, and amino acid analogs in general. Additional non-limiting examples of non-classical amino acids include, but are not limited to: α-aminocaprylic acid, Acpa; (S)-2-aminoethyl-L-cysteine/HCl, Aecys; aminophenylacetate, Afa; 6-amino hexanoic acid, Ahx; γ-amino isobutyric acid and α-aminoisobytyric acid, Aiba; alloisoleucine, Aile; L-allylglycine, Alg; 2-amino butyric acid, 4-aminobutyric acid, and α-aminobutyric acid, Aba; p-aminophenylalanine, Aphe; b-alanine, Bal; p-bromophenylalaine, Brphe; cyclohexylalanine, Cha; citrulline, Cit; β-chloroalanine, Clala; cycloleucine, Cle; p-cholorphenylalanine, Clphe; cysteic acid, Cya; 2,4-diaminobutyric acid, Dab; 3-amino propionic acid and 2,3-diaminopropionic acid, Dap; 3,4-dehydroproline, Dhp; 3,4-dihydroxylphenylalanine, Dhphe; p-flurophenylalanine, Fphe; D-glucoseaminic acid, Gaa; homoarginine, Hag; δ-hydroxylysine/HCl, Hlys; DL-β-hydroxynorvaline, Hnvl; homoglutamine, Hog; homophenylalanine, Hoph; homoserine, Hos; hydroxyproline, Hpr; p-iodophenylalanine, Iphe; isoserine, Ise; a-methylleucine, Mle; DL-methionine-S-methylsulfoniumchloide, Msmet; 3-(1-naphthyl)alanine, 1 Nala; 3-(2-naphthyl) alanine, 2Nala; norleucine, Nie; N-methylalanine, Nimala; Norvaline, Nva; O-benzylserine, Obser; O-benzyltyrosine, Obtyr; O-ethyltyrosine, Oetyr; O-methylserine, Omser; O-methylthreonine, Omthr; O-methyltyrosine, Omtyr; Ornithine, Orn; phenylglycine; penicillamine, Pen; pyroglutamic acid, Pga; pipecolic acid, Pip; sarcosine, Sar; t-butylglycine; t-butylalanine; 3,3,3-trifluroalanine, Tfa; 6-hydroxydopa, Thphe; L-vinylglycine, Vig; (−)-(2R)-2-amino-3-(2-aminoethylsulfonyl) propanoic acid dihydroxochloride, Aaspa; (2S)-2-amino-9-hydroxy-4,7-dioxanonanoic acid, Ahdna; (2S)-2-amino-6-hydroxy-4-oxahexanoic acid, Ahoha; (−)-(2R)-2-amino-3-(2-hydroxyethylsulfonyl) propanoic acid, Ahsopa; (−)-(2R)-2-amino-3-(2-hydroxyethylsulfanyl) propanoic acid, Ahspa; (2S)-2-amino-12-hydroxy-4,7,10-trioxadodecanoic acid, Ahtda; (2S)-2,9-diamino-4,7-dioxanonanoic acid, Dadna; (2S)-2,12-diamino-4,7,10-trioxadodecanoic acid, Datda: (S)-5,5-difluoronorleucine, Dfnl; (S)-4,4-difluoronorvaline, Dfnv; (3R)-1-1-dioxo-1,4thiaziane-3-carboxylic acid, Dtca: (S)-4,4,5,5,6,6,6-heptafluoronorleucine, Hfnl; (S)-5,5,6,6,6-pentafluoronorleucine, Pfnl; (S)-4.4.5.5.5-pentafluoronorvaline, Pfnv; and (3R)-1,4-thiazinane-3-carboxylic acid, Tca. Furthermore, the amino acid can be D (dextrorotary) or L (levorotary). For a review of classical and non-classical amino acids, see Sandberg et al., 1998, J. Med. Chem., 41(14):2481-91.

The DNA endonuclease used in the compositions and methods of the disclosure can be fused to other polypeptide sequences, for example fused to amino acid sequences that encode protein tags (e.g., V5-tag, FLAG-tag, myc-tag, HA-tag, GST-tag, polyHis-tag, MBP-tag), proteins, protein domains, transcription modulators, enzymes acting on small molecule substrates, DNA, RNA and protein modification enzymes (e.g., adenosine deaminase, cytidine deaminase, guanosyl transferase, DNA methyltransferase, RNA methyltransferases, DNA demethylases, RNA demethylases, dioxygenases, polyadenylate polymerases, pseudouridine synthases, acetyltransferases, deacetylase, ubiquitin-ligases, deubiquitinases, kinases, phosphatases, NEDD8-ligases, de-NEDDylases, SUMO-ligases, deSUMOylases, histone deacetylases, histone acetyltransferases histone methyltransferases, histone demethylases), protein DNA binding domains, RNA binding proteins, polypeptide sequences with specific biological functions (e.g., nuclear localization signals, mitochondrial localization signals, plastid localization signals, subcellular localization signals, destabilizing signals, Geminin destruction box motifs), or biological tethering domains (e.g., MS2, Csy4 and lambda N protein).

### 6.4. Nucleic Acids and Host Cells

The disclosure provides nucleic acids (e.g., DNA or RNA) encoding the gRNAs of the disclosure, nucleic acids encoding a DNA endonuclease (e.g., a DNA endonucleiase described in Section 6.3) and pluralities of nucleic acids, for example comprising a nucleic acid encoding a gRNA and a nucleic acid encoding a DNA endonuclease.

A nucleic acid encoding a gRNA can be, for example, a plasmid or a viral genome (e.g., a lentivirus, retrovirus, adenovirus, or adeno-associated virus genome modified to encode the gRNA). Plasmids can be, for example, plasmids for producing virus particles, e.g., lentivirus particles, or plasmids for propagating the gRNA coding sequence in bacterial (e.g., E. coli) or eukaryotic (e.g., yeast) cells.

A nucleic acid encoding a gRNA can, in some embodiments, further encode a DNA endonuclease protein, e.g., a Cas9 protein described in Section 6.3. Alternatively, a DNA endonuclease can be encoded by a separate nucleic acid (e.g., DNA or mRNA). Those of skill in the art will appreciate that plasmids encoding a Cas9 protein can be modified to encode a different Cas9 protein, e.g., a Cas9 variant as described in Section 6.3.

Nucleic acids encoding a DNA endonuclease (e.g., a Cas9 protein) can be codon optimized, e.g., where at least one non-common codon or less-common codon has been replaced by a codon that is common in a host cell. For example, a codon optimized nucleic acid can direct the synthesis of an optimized messenger mRNA, e.g., optimized for expression in a mammalian expression system. As an example, if the intended target nucleic acid is within a human cell, a human codon-optimized polynucleotide encoding Cas9 can be used for producing a Cas9 polypeptide.

Nucleic acids of the disclosure, e.g., plasmids and viral vectors, can comprise one or more regulatory elements such as promoters, enhancers, and other expression control elements (e.g., transcription termination signals, such as polyadenylation signals and poly-U sequences). Such regulatory elements are described, for example, in Goeddel, 1990, GENE EXPRESSION TECHNOLOGY: METHODS IN ENZYMOLOGY 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as retinal tissue (e.g., by using a RHO promoter), or in particular cell types (e.g., retinal photoreceptor cells). Regulatory elements may also direct expression in a temporal-dependent manner, such as in a cell-cycle dependent or developmental stage-dependent manner, which may or may not also be tissue or cell-type specific. In some embodiments, a nucleic acid of the disclosure comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof, e.g., to express a gRNA and a Cas9 protein separately. Examples of pol III promoters include, but are not limited to, U6 and H1 promoters. Examples of pol II promoters include, but are not limited to, the retroviral Rous Sarcoma virus (RSV) LTR promoter (optionally with the RSV enhancer), the cytomegalovirus (CMV) promoter (optionally with the CMV enhancer) (see, e.g., Boshart et al, Cell, 1985, 41:521-530), the SV40 promoter, the dihydrofolate reductase promoter, the β-actin promoter, the phosphoglycerol kinase (PGK) promoter, and the EF1a promoter. Exemplary enhancer elements include WPRE; CMV enhancers; the R-U5′ segment in LTR of HTLV-I; SV40 enhancer; and the intron sequence between exons 2 and 3 of rabbit β-globin. It will be appreciated by those skilled in the art that the design of an expression vector can depend on such factors as the choice of the host cell, the level of expression desired, etc.

A polynucleotide encoding a guide RNA, a DNA endonuclease, and/or any additional nucleic acid or proteinaceous molecule advantageous for carrying out the various aspects of the methods disclosed herein can be comprised within vector (e.g., a recombinant expression vector).

The term “vector” refers to a polynucleotide molecule capable of transporting another nucleic acid to which it has been linked. One type of polynucleotide vector includes a “plasmid”, which refers to a circular double-stranded DNA loop into which additional nucleic acid segments are or can be ligated. Another type of polynucleotide vector is a viral vector; wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operatively linked. Such vectors can be referred to herein as “recombinant expression vectors”, or more simply “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Vectors can include, but are not limited to, viral vectors based on vaccinia virus, poliovirus, adenovirus, adeno-associated virus (e.g., AAV2, AAV5, AAV7m8, AAV8), SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXTI, pSG5, pSVK3, pBPV, pMSG, and pSVLSV40 (Pharmacia). Additional vectors contemplated for eukaryotic target cells include, but are not limited to, the vectors pCTx-I, pCTx-2, and pCTx-3. Other vectors can be used so long as they are compatible with the host cell.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. The vector can be a self-inactivating vector that either inactivates the viral sequences or the components of the CRISPR machinery or other elements.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-I promoter (EF1), e.g., EF1 alpha short promoter, a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing guide RNAs, various promoters such as RNA polymerase III promoters, including for example U6 and H1, can be advantageous. Descriptions of and parameters for enhancing the use of such promoters are known in art; see, e.g., Ma, et. al., 2014, Molecular Therapy—Nucleic Acids 3, el 61. In some embodiments, a U6 promoter is used to drive expression of a gRNA. In other embodiments, a H1 promoter is used to drive expression a g RNA.

An expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter, an EF1 alpha promoter, e.g., EF1 alpha short (EFS) promoter). In some cases, the promoter can be a spatially restricted and/or temporally restricted promoter (e.g., a tissue specific promoter, a cell type specific promoter, etc.). In some embodiments, a RHO promoter is used to drive expression of a Cas9 protein. In other embodiments, a hGRK1 promoter is used to drive expression of a Cas9 protein.

The disclosure also provides a host cell comprising a nucleic acid of the disclosure. Such host cells can be used, for example, to produce virus particles encoding a gRNA of the disclosure and, optionally, a DNA endonuclease such as a Cas9 protein. In some embodiments, host cells are used to produce virus particles encoding a gRNA (but no Cas9 protein) and to produce virus particles encoding a Cas9 protein (but no gRNA9). The virus particles encoding a gRNA and the virus particles encoding a Cas9 can then be used together to deliver a gRNA and Cas9 to a cell. Host cells can also be used to make vesicles containing a gRNA and, optionally, a DNA endonuclease such as a Cas9 protein (e.g., as described in Montagna et al., 2018, Molecular Therapy: Nucleic Acids, 12:453-462). Exemplary host cells include eukaryotic cells, e.g., mammalian cells. Exemplary mammalian host cells include human cell lines such as BHK-21, BSRT7/5, VERO, WI38, MRCS, A549, HEK293, HEK293T, Caco-2, B-50 or any other HeLa cells, HepG2, Saos-2, HuH7, and HT1080 cell lines. Host cells can be engineered host cells, for example, host cells engineered to express a DNA binding protein such a repressor (e.g., TetR), to regulate virus or vesicle production (see Petris et al., 2017, Nature Communications, 8:15334).

Host cells can also be used to propagate the gRNA coding sequences of the disclosure. The host cell can be a eukaryote or prokaryote and includes, for example, yeast (such as Pichia pastoris or Saccharomyces cerevisiae), bacteria (such as E. coli or Bacillus subtilis), insect Sf9 cells (such as baculovirus-infected SF9 cells) or mammalian cells (such as Human Embryonic Kidney (HEK) cells, Chinese hamster ovary cells, HeLa cells, human 293 cells and monkey COS-7 cells).

### 6.5. Systems, Particles, and Cells

The disclosure further provides systems comprising a gRNA of the disclosure and a DNA endonuclease such as a Cas9 protein. The systems can comprise a ribonucleoprotein particle (RNP) in which the gRNA as described herein is complexed with a DNA endonuclease such as a Cas9 protein. The Cas9 protein can be, for example, a Cas9 protein described in Section 6.3. Systems of the disclosure can further comprise genomic DNA complexed with the gRNA and the DNA endonuclease. Accordingly, the disclosure provides a system comprising a gRNA of the disclosure, a genomic DNA comprising a RHO gene having a P347 mutation, and a DNA endonuclease such as Cas9, all complexed with one another.

The systems of the disclosure can exist within a cell (whether the cell is in vivo, ex vivo, or in vitro) or outside a cell (e.g., in a particle our outside of a particle).

The disclosure further provides particles comprising a gRNA of the disclosure and provides particles comprise a nucleic acid encoding a gRNA of the disclosure. The particles can further comprise a DNA endonuclease such as a Cas9 protein, e.g., a Cas9 protein described in Section 6.3, or a nucleic acid encoding the Cas9 protein (e.g., DNA or mRNA). Exemplary particles include lipid nanoparticles, vesicles, and gold nanoparticles. In some embodiments, a particle contains only a single species of gRNA.

The disclosure further provides particles (e.g., virus particles) comprising a nucleic acid encoding a gRNA of the disclosure. The particles can further comprise a nucleic acid encoding a Cas9 protein.

The disclosure further provides pluralities of particles (e.g., pluralities of virus particles). Such pluralities can include a particle encoding a gRNA and a different particle encoding a Cas9 protein. For example, a plurality of particles can comprise a virus particle (e.g., a AAV2, AAV5, AAV7m8, or AAV8 virus particle) encoding a gRNA and a second virus particle (e.g., a AAV2, AAV5, AAV5, AAV7m8, or AAV8 virus particle) encoding a Cas9 protein.

The disclosure further provides cells and populations of cells (e.g., a population comprising 10 or more, 50 or more 100 or more, 1,000 or more, or 100,000 thousand or more cells) comprising a gRNA of the disclosure. Such cells and populations can further comprise a DNA endonuclease such as a Cas9 protein or a nucleic acid encoding the Cas9 protein (e.g., DNA or mRNA). In some embodiments, such cells and populations are isolated, e.g., isolated from cells not containing the gRNA. The cells and populations of cells can be, for example, human cells such as a iPSC, retinal cell, photoreceptor cell, retinal progenitor cell, etc.

The cell populations of the disclosure can be cells in which gene editing by the systems of the disclosure has taken place, or cells in which the components of a system of the disclosure have been expressed but gene editing has not taken place, or a combination thereof. A cell population can comprise, for example, a population in which at least 20%, at least 30%, at least 40%, at least 50%, at least 60%, or at least 70% of the cells have undergone gene editing by a system of the disclosure.

In the systems, particles, cells and cell populations of the disclosure comprising a Cas9 protein, the Cas9 protein should be a Cas9 protein capable of recognizing a PAM near (e.g., adjacent) to the target sequence corresponding to the gRNA's spacer sequence.

### 6.6. Pharmaceutical Compositions

Also disclosed herein are pharmaceutical formulations and medicaments comprising a g RNA, nucleic acid or plurality of nucleic acids, system, particle, or plurality of particles of the disclosure together with a pharmaceutically acceptable excipient.

Suitable excipients include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, antinfective agents, suspending agents, wetting agents, viscosity modifiers, tonicity agents, stabilizing agents, and other components and combinations thereof. Suitable pharmaceutically acceptable excipients can be selected from materials which are generally recognized as safe (GRAS), and may be administered to an individual without causing undesirable biological side effects or unwanted interactions. Suitable excipients and their formulations are described in Remington's Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions.

The components of the pharmaceutical formulation can be dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol.

In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers.

In some embodiments, the formulation can be distributed or packaged in a liquid form, or alternatively, as a solid, obtained, for example by lyophilization of a suitable liquid formulation, which can be reconstituted with an appropriate carrier or diluent prior to administration. In some embodiments, the formulations can comprise a guide RNA and a DNA endonuclease in a pharmaceutically effective amount sufficient to edit a rho gene having a P347 mutation in a cell. In some embodiments, the formulations can comprise a guide RNA and a DNA endonuclease in a pharmaceutically effective amount sufficient to treat retinitis pigmentosa. The vaccine formulation can be formulated for medical and/or veterinary use.

### 6.7. Methods of Altering a Cell

The disclosure further provides methods of using the gRNAs, nucleic acids (including pluralities of nucleic acids), systems, and particles (including pluralities of particles) of the disclosure for altering cells. Methods of the disclosure can be used, for example, to treat subjects having a RP caused by a P347 mutation in their RHO gene, for example a P347L mutation.

In one aspect, a method of altering a cell comprises contacting a human cell having a RHO gene with a P347L mutation with a nucleic acid, particle, system or pharmaceutical composition described herein.

Contacting a cell with a disclosed nucleic acid, particle, system or pharmaceutical composition can be achieved by any method known in the art and can be performed in vivo, ex vivo, or in vitro. In some embodiments, the methods can include obtaining one or more cells from a subject prior to contacting the cell(s) with a herein disclosed nucleic acid, particle, system or pharmaceutical composition. In some embodiments, the methods can further comprise returning or implanting the contacted cell or a progeny thereof to the subject.

gRNAs and endonucleases, as well as nucleic acids encoding gRNAs and nucleic acids encoding endonucleases can be delivered to a cell by any means known in the art, for example, by viral or non-viral delivery vehicles, electroporation or lipid nanoparticles. DNA endonucleases can be delivered to a cell as one or more polypeptides, either alone or pre-complexed with one or more guide RNAs, or as a nucleic acid (DNA or RNA) encoding the DNA endonuclease.

Polynucleotides, such as gRNA and/or a polynucleotide encoding an endonuclease, can be delivered to a cell (ex vivo or in vivo) by a lipid nanoparticle (LNP). LNPs can have, for example, a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm. LNPs can be made from cationic, anionic, neutral lipids, and combinations thereof. Neutral lipids, such as the fusogenic phospholipid DOPE or the membrane component cholesterol, can be included in LNPs as ‘helper lipids’ to enhance transfection activity and nanoparticle stability.

LNPs can also be comprised of hydrophobic lipids, hydrophilic lipids, or both hydrophobic and hydrophilic lipids. Lipids and combinations of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2- DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerCl4, and PEG-CerC20. Lipids can be combined in any number of molar ratios to produce a LNP. In addition, the polynucleotide(s) can be combined with lipid(s) in a wide range of molar ratios to produce a LNP.

gRNAs and/or DNA endonucleases can be delivered to a cell via an adeno-associated viral vector (e.g., of an AAV2, AAV5, AAV7m8, AAV8, AAV9, or AAVrh8r serotype), or by another viral vector. Other viral vectors include, but are not limited to lentivirus, adenovirus, alphavirus, enterovirus, pestivirus, baculovirus, herpesvirus, Epstein Barr virus, papovavirusr, poxvirus, vaccinia virus, and herpes simplex virus. In some embodiments, a Cas9 mRNA is formulated in a lipid nanoparticle, while a sgRNA is delivered to a cell in an AAV or other viral vector. In some embodiments, one or more AAV vectors (e.g., one or more AAV2, AAV5, AAV7m8, or AAV8 viral vectors) are used to deliver both a sgRNA and a Cas9. In some embodiments, a sgRNA and a Cas9 are delivered using separate vectors (e.g., when the Cas9 is SpCas9). In other embodiments, a sgRNA and a Cas9 are delivered using a single vector (e.g., when the Cas9 is Nme2Cas9). Nme2Cas9, with it's relatively small size, can be delivered with a gRNA (e.g., sgRNA) using a single AAV vector.

Compositions and methods for administering gRNAs and endonucleases to a cell and/or subject are further described in PCT Patent Application Publication WO 2019/102381, which is incorporated by reference herein in its entirety.

In some embodiments, the methods result in cleavage of a RHO gene encoding a P347 mutation. In some embodiments, the methods result in preferential cleavage of a RHO gene encoding a P347 mutation, as compared to a RHO gene encoding a wild-type RHO polypeptide (e.g., a RHO gene not having a P347 mutation). The degree of preference can be quantitated, for example, by measuring the percentage editing of a RHO gene having a P347 mutation and the percentage editing of a RHO gene not having a P347 mutation in a population of cells each containing a RHO gene having a P347 mutation and a RHO gene not having a P347 mutation. In some embodiments, the preferential cleavage of the RHO gene having a P347 mutation over cleavage of a RHO gene not having a P347 mutation is by a factor of at least 1.3, at least 1.5, at least 2, at least 2.5, at least 3, at least 4, at least 5, at least 10, at least 11, or at least 100. In some embodiments, the preferential cleavage of the RHO gene having a P347 mutation over cleavage of the RHO gene not having a P347 mutation can be by a factor of 1.3 to 100, 2 to 100, 5 to 100, 10 to 100, 1.3-11, 2-11, 2.5-11, 3-11, 4-11, 5-11, 1.1-5, 1.3-5, 2-10, 3-10, 4-10, 5-10, 2-4, 2-5, 2-4, 3-5, or 4-5.

DNA cleavage can result in a single-strand break (SSB) or double-strand break (DSB) at particular locations within the DNA molecule. Such breaks can be and regularly are repaired by natural, endogenous cellular processes, such as homology-dependent repair (HDR) and non-homologous end-joining (NHEJ). These repair processes can edit the targeted polynucleotide by introducing a mutation, thereby resulting in a polynucleotide having a sequence which differs from the polynucleotide's sequence prior to cleavage by a DNA endonuclease.

NHEJ and HDR DNA repair processes consist of a family of alternative pathways. Non-homologous end-joining (NHEJ) refers to the natural, cellular process in which a double-stranded DNA-break is repaired by the direct joining of two non-homologous DNA segments. See, e.g. Cahill et al., 2006, Front. Biosci. 11:1958-1976. DNA repair by non-homologous end-joining is error-prone and frequently results in the untemplated addition or deletion of DNA sequences at the site of repair. Thus, NHEJ repair mechanisms can introduce mutations into the coding sequence which can disrupt gene function. NHEJ directly joins the DNA ends resulting from a double-strand break, sometimes with a modification of the polynucleotide sequence such as a loss of or addition of nucleotides in the polynucleotide sequence. The modification of the polynucleotide sequence can disrupt (or perhaps enhance) gene expression.

Homology-dependent repair (HDR) utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus.

A third repair mechanism includes microhomology-mediated end joining (MMEJ), also referred to as “Alternative NHEJ (ANHEJ)”, in which the genetic outcome is similar to NHEJ in that small deletions and insertions can occur at the cleavage site. MMEJ can make use of homologous sequences of a few base pairs flanking the DNA break site to drive a more favored DNA end joining repair outcome. In some instances, it may be possible to predict likely repair outcomes based on analysis of potential microhomologies at the site of the DNA break.

Modifications of a cleaved polynucleotide by HDR, NHEJ, and/or ANHEJ can result in, for example, mutations, deletions, alterations, integrations, gene correction, gene replacement, gene tagging, transgene insertion, nucleotide deletion, gene disruption, translocations and/or gene mutation. The aforementioned process outcomes are examples of editing a polynucleotide.

Accordingly, in some embodiments, the contacting step of the methods of the disclosure results in the editing of a RHO gene comprising a P347 mutation. For example, the editing of the RHO gene comprising a P347 mutation can include deletion, insertion, or substitution of one or more nucleotides in the RHO gene.

The methods can provide for advantageous and/or therapeutic results in the cell and/or the subject in which the cell is located. In some embodiments, the methods can reduce expression of the RHO gene comprising a P347 mutation. Thus, the methods can reduce the amount of RHO protein comprising a P347 mutation within the contacted cell. In some embodiments, the methods can decrease the amount of misfolded RHO protein within the cell. In some embodiments, the methods can decrease the amount of mislocalized RHO protein within the cell. In some embodiments, the methods can decrease the rate of or amount of cell death. In some embodiments, the methods can delay, slow progression, halt, or reverse onset of a RHO-associated disease such as retinitis pigmentosa (RP).

In one aspect, the disclosure provides methods for treating a subject having a P347 mutation using the gRNAs, nucleic acids, systems, and particles of the disclosure. The methods can comprise editing a RHO gene in one or more cells from the subject or one or more cells derived from a cell of the subject (e.g., an induced pluripotent stem cell (iPSC)). For example, one or more cells from the subject or one or more cells derived from a cell of the subject can be contacted with a gRNA, nucleic acid, system, or particle of the disclosure ex vivo, and cells having an edited RHO gene or progeny thereof can subsequently be implanted into the subject. iPSCs can be generated from epithelial cells of a subject by technologies known to the skilled artisan. The chromosomal DNA of such iPSC cells can be edited using the materials and methods described herein. More specifically, a single- or double-strand break within or near a P347 mutation in the RHO gene can be induced by DNA cleavage (e.g., by a DNA endonuclease). Repair of the cleaved DNA (e.g., by insertion, deletion, substitution, or frameshift mutations) can result in editing of the RHO gene at the site of the single- or double-strand break. Edited iPSCs can subsequently be differentiated, for instance into photoreceptor cells or retinal progenitor cells. In some embodiments, resultant differentiated cells can be implanted into the subject.

In some aspects of the methods, differentiated cells of subject can be used. For example, photoreceptor cells or retinal progenitor cells can be used (e.g., following isolation from the subject). In such methods, implantation of edited cells can proceed without an intervening differentiation step.

Advantages of ex vivo cell therapy approaches include the ability to conduct a comprehensive analysis of the therapeutic prior to administration. Nuclease-based therapeutics can have some level of off-target effects. Performing gene correction ex vivo allows a method user to characterize the corrected cell population prior to implantation, including identifying any undesirable off-target effects. Where undesirable effects are observed, a method user may opt not to implant the cells or cell progeny, may further edit the cells, or may select new cells for editing and analysis. Other advantages include ease of genetic correction in iPSCs compared to other primary cell sources. iPSCs are prolific, making it easy to obtain the large number of cells that will be required for a cell-based therapy. Furthermore, iPSCs are an ideal cell type for performing clonal isolations. This allows screening for the correct genomic correction, without risking a decrease in viability.

In another aspect, the disclosure provides in vivo methods for treating a subject with a P347 mutation. In some aspects, the method is an in vivo cell-based therapy. Chromosomal DNA of the cells in the subject can be edited using the materials and methods described herein. For example, the in vivo method can comprise editing a P347 mutation in a RHO gene in a cell of a subject, such as photoreceptor cells or retinal progenitor cells. In some embodiments, the in vivo methods comprise administering a pharmaceutical composition of the disclosure to or near the eye of a subject, e.g., by sub-retinal injection or intravitreal injection.

Although certain cells present an attractive target for ex vivo treatment and therapy, increased efficacy in delivery may permit direct in vivo delivery to such cells. Ideally the targeting and editing is directed to the relevant cells. Cleavage in other cells can also be prevented by the use of promoters only active in certain cell types and/or developmental stages.

Additional promoters are inducible, and therefore can be temporally controlled if the nuclease is delivered as a plasmid. The amount of time that delivered RNA and protein remain in the cell can also be adjusted using treatments or domains added to change the half-life. In vivo treatment would eliminate a number of treatment steps, but a lower rate of delivery can require higher rates of editing. In vivo treatment can eliminate problems and losses from ex vivo treatment and engraftment.

An advantage of in vivo gene therapy can be the ease of therapeutic production and administration. Administration can be, for example, by sub-retinal injection of a pharmaceutical composition. The same therapeutic approach and therapy has the potential to be used to treat more than one patient, for example a number of patients who share the same or similar genotype or allele. In contrast, ex vivo cell therapy typically requires using a subject's own cells, which are isolated, manipulated and returned to the same patient.

For ameliorating a disorder associated with RHO (e.g., retinitis pigmentosa), the principal targets for gene editing can be within a cell such as a human cell. For example, in an ex vivo method, human cells can be somatic cells, which after being modified using techniques described herein, can give rise to differentiated cells, e.g., photoreceptor cells or retinal progenitor cells. In an in vivo method, human cells can be photoreceptor cells or retinal progenitor cells. By performing gene editing in autologous cells that are derived from and therefore already completely matched with a subject, it is possible to generate cells that can be safely re-introduced into the subject, and effectively give rise to a population of cells that can be effective in ameliorating one or more clinical conditions associated with the subject's disease.

Progenitor cells (also referred to as stem cells herein) are capable of both proliferation and giving rise to more progenitor cells, which in turn have the ability to generate a large number of cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell can derive from a multipotent cell that itself is derived from a multipotent cell, and so on. While each of these multipotent cells can be considered stem cells, the range of cell types that each can give rise to can vary considerably. Some differentiated cells also have the capacity to give rise to cells of greater developmental potential. Such capacity can be natural or can be induced artificially upon treatment with various factors. In many biological instances, stem cells can also be “multipotent” because they can produce progeny of more than one distinct cell type, but this is not required.

Human cells described herein can be induced pluripotent stem cells (iPSCs). An advantage of using iPSCs in the methods of the disclosure is that the cells can be derived from the same subject to which the progenitor cells are to be administered. That is, a somatic cell can be obtained from a subject, reprogrammed to an induced pluripotent stem cell, and then differentiated into a progenitor cell to be administered to the subject (e.g., an autologous cell). Because progenitors are essentially derived from an autologous source, the risk of engraftment rejection or allergic response can be reduced compared to the use of cells from another subject or group of subjects. In addition, the use of iPSCs negates the need for cells obtained from an embryonic source. Thus, in one aspect, the stem cells used in the disclosed methods are not embryonic stem cells.

Methods are known in the art that can be used to generate pluripotent stem cells from somatic cells. Pluripotent stem cells generated by such methods can be used in the method of the disclosure.

Reprogramming methodologies for generating pluripotent cells using defined combinations of transcription factors have been described. Mouse somatic cells can be converted to ES cell-like cells with expanded developmental potential by the direct transduction of Oct4, Sox2, Klf4, and c-Myc; see, e.g., Takahashi and Yamanaka, 2006, Cell 126(4): 663-76. iPSCs resemble ES cells, as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission (see, e.g., Maherali and Hochedlinger, 2008, Cell Stem Cell. 3(6):595-605), and tetraploid complementation.

Human iPSCs can be obtained using similar transduction methods, and the transcription factor trio, OCT4, SOX2, and NANOG, has been established as the core set of transcription factors that govern pluripotency; see, e.g., 2014, Budniatzky and Gepstein, Stem Cells Transl Med. 3(4):448-57; Barrett et al, 2014, Stem Cells Trans Med 3: 1-6 sctm.2014-0121; Focosi et al, 2014, Blood Cancer Journal 4: e211. The production of iPSCs can be achieved by the introduction of nucleic acid sequences encoding stem cell-associated genes into an adult, somatic cell, historically using viral vectors.

iPSCs can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., 2010, Cell Stem Cell, 7(5):618-30. Reprogramming can be achieved by introducing a combination of nucleic acids encoding stem cell-associated genes, including, for example, Oct-4 (also known as Oct-3/4 or Pouf51), SoxI, Sox2, Sox3, Sox 15, Sox 18, NANOG, KlfI, Klf2, Klf4, Klf5, NR5A2, c-Myc, 1-Myc, n-Myc, Rem2, Tert, and LIN28. Reprogramming using the methods and compositions described herein can further comprise introducing one or more of Oct-3/4, a member of the Sox family, a member of the Klf family, and a member of the Myc family to a somatic cell. The methods and compositions described herein can further comprise introducing one or more of each of Oct-4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one aspect the reprogramming is not affected by a method that alters the genome. Thus, in such examples, reprogramming can be achieved, e.g., without the use of viral or plasmid vectors.

Efficiency of reprogramming (the number of reprogrammed cells) derived from a population of starting cells can be enhanced by the addition of various agents, e.g., small molecules, as shown by Shi et al., 2008, Cell-Stem Cell 2:525-528; Huangfu et al., 2008, Nature Biotechnology 26(7):795-797; and Marson et al., 2008, Cell-Stem Cell 3: 132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HD AC) inhibitors, valproic acid, 5′-azacytidine, dexamethasone, suberoylanilide, hydroxamic acid (SAHA), vitamin C, and trichostatin (TSA), among others. Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-IH,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pi valoyloxy methyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylureido)caproic hydroxamic acid), AOE (2-amino-8-oxo-9, 10-epoxy decanoic acid), CHAP31 and CHAP 50. Other reprogramming enhancing agents include, for example, dominant negative forms of the HDACs (e.g, catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Titan Pharmaceuticals, MethylGene, and Sigma Aldrich.

To confirm the induction of pluripotent stem cells, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbxl5, Ecatl, Esgl, Eras, Gdfi, Fgf4, Cripto, Daxl, Zpf296, Slc2a3, Rexl, Utfl, and Natl. In one case, for example, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. Detection can involve not only RT-PCR, but also detection of protein markers. Intracellular markers can be best identified via RT-PCR, or protein detection methods such as immunocytochemistry, while cell surface markers are readily identified, e.g., by immunocytochemistry.

Pluripotency of isolated cells can be confirmed by tests evaluating the ability of the iPSCs to differentiate into cells of each of the three germ layers. As one example, teratoma formation in nude mice can be used to evaluate the pluripotent character of the isolated clones. The cells can be introduced into nude mice and histology and/or immunohistochemistry can be performed on a tumor arising from the cells. The growth of a tumor comprising cells from all three germ layers, for example, further indicates that the cells are pluripotent stem cells.

In some examples, the cells used in the method described herein are photoreceptor cells or retinal progenitor cells (RPCs). RPCs are multipotent progenitor cells that can give rise to all the six neurons of the retina as well as the Muller glia. Muller glia are a type of retinal glial cells and are the major glial component of the retina. Their function is to support the neurons of the retina and to maintain retinal homeostasis and integrity. Muller glia isolated from adult human retinas have been shown to differentiate into rod photoreceptors. Functional characterization of such Muller glia-derived photoreceptors by patch-clamp recordings has revealed that their electrical properties are comparable to those of adult rods (Giannelli et al, 2011, Stem Cells, (2):344-56). RPCs are gradually specified into lineage-restricted precursor cells during retinogenesis, which then maturate into the terminally differentiated neurons or Muller glia. Fetal-derived human retinal progenitor cells (hRPCs) exhibit molecular characteristics indicative of a retinal progenitor state up to the sixth passage. They demonstrate a gradual decrease in the percentages of KI67−, SOX2−, and vimentin-positive cells from passages 1 to 6, whereas a sustained expression of nestin and PAX6 is seen through passage 6.

Microarray analysis of passage 1 hRPCs demonstrate the expression of early retinal developmental genes: VIM (vimentin), KI67, NES (nestin), PAX6, SOX2, HES5, GNL3, OTX2, DACH1, SIX6, and CHX10 (VSX2). The hRPCs are functional in nature and respond to excitatory neurotransmitters (Schmitt et al., 2009, Investigative Ophthalmology and Visual Sciences. 50(12):5901-8). The outermost region of the retina contains a supportive retinal pigment epithelium (RPE) layer, which maintains photoreceptor health by transporting nutrients and recycling shed photoreceptor parts. The RPE is attached to Bruch's membrane, an extracellular matrix structure at the interface between the choroid and retina. On the other side of the RPE, moving inwards towards the interior of the eye, there are three layers of neurons: light sensing rod and cone photoreceptors, a middle layer of connecting neurons (amacrine, bipolar and horizontal cells) and the innermost layer of ganglion cells, which transmit signals originating in the photoreceptor layer through the optic nerve and into the brain. In some aspects, the cells described herein are photoreceptor cells, which are specialized types of neurons found in the retina. Photoreceptors convert light into signals that are able to stimulate biological processes and are responsible for sight. Rods and cones are the two classic photoreceptor cells that contribute information to the visual system.

Retinal cells, including progenitor cells may be isolated according to any method known in the art. For example, retinal cells can be isolated from fresh surgical specimens. The retinal pigment epithelium (RPE) can be separated from the choroid by digesting the tissue with type IV collagenase and the retinal pigment epithelium patches can be cultured. Following the growth of 100-500 cells from the explant, the primary cultures can be passaged (Ishida M. et al., 1998, Current Eye Research, 17(4):392-402) and characterized for expression of RPE markers. Rods can be isolated by disruption of the biopsied retina using papain. Precautions can be taken to avoid a harsh disruption and improve cell yield. The isolated cells can be sorted to yield a population of pure rod cells and characterized further by immunostaining (Feodorova et al., 2015, MethodsX, 2:39-46).

To isolate cones, the neural retina can be identified, cut-out, and placed on 10% gelatin. The inner retinal layers can be isolated using a laser. The isolated cone monolayers can be cultured for 18 hours and compared with untreated retinas by light microscopy and transmission microscopy to check for any structural damage. The cells can be characterized for expression of cone-specific markers (Salchow et al., 2001, Current Eye Research, 22 (2):85-9).

To isolate retinal progenitor cells, the biopsied retina can be minced with dual scalpels and digested enzymatically in an incubator at 37° C. The supernatants of the digested cells can be centrifuged and the cells can be resuspended in cell-free retinal progenitor-conditioned medium. The cells can be transferred to fibronectin-coated tissue culture flasks containing fresh media and cultured (Klassen et al., 2004, Journal of Neuroscience Research, 77:334-343).

Patient-specific iPS cells or cell line can be created. There are many established methods in the art for creating patient specific iPS cells, e.g., as described in Takahashi and Yamanaka 2006; Takahashi, Tanabe et al. 2007. For example, the creating step can comprise: a) isolating a somatic cell, such as a skin cell or fibroblast, from the patient; and b) introducing a set of pluripotency-associated genes into the somatic cell in order to induce the cell to become a pluripotent stem cell. The set of pluripotency-associated genes can be one or more of the genes selected from the group consisting of OCT4, SOX1, SOX2, SOX3, SOX15, SOX18, NANOG, KLF1, KLF2, KLF4, KLF5, c-MYC, n-MYC, REM2, TERT and LIN28.

In some aspects, a biopsy or aspirate of a subject's bone marrow can be performed. A biopsy or aspirate is a sample of tissue or fluid taken from the body. There are many different kinds of biopsies or aspirates. Nearly all of them involve using a sharp tool to remove a small amount of tissue. If the biopsy will be on the skin or other sensitive area, numbing medicine can be applied first. A biopsy or aspirate can be performed according to any of the known methods in the art. For example, in a bone marrow aspirate, a large needle is used to enter the pelvis bone to collect bone marrow.

In some aspects, a mesenchymal stem cell can be isolated from a subject. Mesenchymal stem cells can be isolated according to any method known in the art, such as from a subject's bone marrow or peripheral blood. For example, marrow aspirate can be collected into a syringe with heparin. Cells can be washed and centrifuged on a Percoll™ density gradient. Cells, such as blood cells, liver cells, interstitial cells, macrophages, mast cells, and thymocytes, can be separated using density gradient centrifugation media, Percoll™ The cells can then be cultured in Dulbecco's modified Eagle's medium (DMEM) (low glucose) containing 10% fetal bovine serum (FBS) (Pittinger et. al., 1999, Science 284: 143-147).

The methods of the present disclosure can also comprise differentiating genome-edited iPSCs into photoreceptor cells or retinal progenitor cells. The differentiating step may be performed according to any method known in the art. For example, iPSCs can be used to generate retinal organioids and photoreceptors as described in the art (Phillips et al., 2014, Stem Cells, 32(6): pgs. 1480-1492; Zhong et al., 2014, Nat. Commun., 5:4047; Tucker et al., 2011, PLoS One, 6(4): e18992). For example, hiPSC can be differentiated into retinal progenitor cells using various treatments, including Wnt, Nodal, and Notch pathway inhibitors (Noggin, Dkl, Lefty A, and DAPT) and other growth factors. The retinal progenitor cells can be further differentiated into photoreceptor cells, the treatment including: exposure to native retinal cells in coculture systems, RX+ or Mitf+by subsequent treatment with retinoic acid and taurine, or exposure to several exogenous factors including Noggin, Dkkl, DAPT, and insulin-like growth factor (Yang et al., 2016, Stem Cells International 2016).

The methods of the present disclosure can also comprise differentiating the genome-edited mesenchymal stem cells into photoreceptor cells or retinal progenitor cells. The differentiating step can be performed according to any method known in the art.

The methods of the present disclosure can also comprise implanting the photoreceptor cells or retinal progenitor cells into a subject. This implanting step can be accomplished using any method of implantation known in the art. For example, cells can be injected directly in the subject's blood or otherwise administered to the subject.

Another aspect of the methods can include implanting edited photoreceptor cells or retinal progenitor cells into a subject. The implanting step can be accomplished using any method of implantation known in the art. For example, the genetically modified cells can be injected directly in the subject's eye or otherwise administered to the patient.

### 7. EXAMPLES

### 7.1. Example 1: SpCas9 and Nme2Cas9 Editing of RHO P347L Gene

7.1.1. Materials and Methods

7.1.1.1. Plasmids and Oligonucleotides

SpCas9 and its high-fidelity variants (variant E containing the K526E mutation, variant ES containing the K526E+R661S mutations, variant ESN containing the K526E+R661S+Y515N mutations, evoCas9 containing the K526E+R661S+Y515N+M495V mutations) and Nme2Cas9 were expressed from plasmids containing a CBh-driven expression cassette. All sgRNAs were expressed from a pUC19 plasmid containing U6-driven Pol III expression cassettes (pUC19-sgRNA). The spacer sequences of the sgRNAs and the oligonucleotides used to generate the expression constructs are reported in Table 5. Spacers were cloned as annealed oligonucleotides into a double Bbsl site of the pUC19-sgRNA plasmids containing the corresponding constant sgRNA region for SpCas9 (SEQ ID NO:176) (3′ sgRNA sequence 8 as set forth in Table 3) or Nme2Cas9 (SEQ ID NO:193) (3′ sgRNA sequence 1 as set forth in Table 4).

A third generation SIN lentiviral transfer vector expressing wild-type SpCas9 under the control of an EF-1a promoter and Guide1 sgRNA targeting the RHO P347L mutation under the control of a U6 promoter, as well as a puromycin resistance cassette, was generated for transduction studies.

The human rhodopsin (RHO) gene was PCR-amplified using the primers RHO_gene-F and RHO_gene-R (Table 6) from genomic DNA extracted from HEK293T/17 cells and cloned into a plasmid containing a CMV-driven expression cassette, generating the pCMV-RHO-wt plasmid. The RHO P347L mutation was introduced in this construct by site-directed mutagenesis using primers Mut_P347L-F and Mut_P347L-R reported in Table 6, generating the pCMV-RHO-P347L plasmid. The cDNA for wt RHO and P347L RHO was obtained by reverse transcription using the Revertaid RT Reverse Transcription Kit (ThermoFisher Scientific) of total RNA extracted from HEK293T/17 cells transfected either with pCMV-RHO-wt or pCMV-RHO-P347L, respectively, and was amplified using the primers RHO_cDNA-F and RHO_cDNA-R (Table 6) for cloning into a lentiviral vector for the production of stable cell lines, generating lentiRHO-wt and lentiRHO-P347L. The two cDNAs included also part of the 3′-UTR sequence of the RHO gene. In addition, these vectors contained a blasticidin selection marker. All PCR products used for preparing plasmid constructs were generated using the Phusion high-fidelity DNA polymerase (ThermoFisher Scientific). All oligonucleotides were obtained from Eurofins Genomics.

TABLE 5
sgRNA spacers, corresponding RHO genomic sequences and cloning
oligonucleotides

| | | corresponding RHO | | |
|---|---|---|---|---|
| Name | spacer | genomic sequence* | cloning oligo 1 | cloning oligo 2 |
| Guide 1 | GGUCUUAGGC | gcaGGTCTTAGGCCa | caccGGTCTTAGG | aaacGGTGGCCCt |
| SpCas9 | CaGGGCCACC | GGGCCACCTGGctc | CCaGGGCCACC | GGCCTAAGACC |
| P347L | (SEQ ID NO: 5) | (SEQ ID NO: 220) | (SEQ ID NO: 233) | (SEQ ID NO: 246) |
| Guide 2 | GCCCUGGCCU | gtgGCCCtGGCCTAA | caccGCCCtGGCCT | aaacAGGCAGGTC |
| SpCas9 | AAGACCUGCC | GACCTGCCTAGGact | AAGACCTGCCT | TTAGGCCaGGGC |
| P347L | U (SEQ ID | (SEQ ID NO: 221) | (SEQ ID NO: 234) | (SEQ ID NO: 247) |
| | NO: 6) | | | |
| Guide 3 | CCUAGGCAGG | agtCCTAGGCAGGTC | caccgCCTAGGCAG | aaacTGGCCTAAG |
| SpCas9 | UCUUAGGCCA | TTAGGCCaGGGcca | GTCTTAGGCCA | ACCTGCCTAGGc |
| P347L | (SEQ ID NO: 7) | (SEQ ID NO: 222) | (SEQ ID NO: 235) | (SEQ ID NO: 248) |
| Guide 1 | GGUCUUAGGC | gcaGGTCTTAGGCCG | caccGGTCTTAGG | aaacGGT GGCCCC |
| SpCas9 | CGGGGCCACC | GGGCCACCTGGctc | CCGGGGCCACC | GGCCTAAGACC |
| P347wt | (SEQ ID | (SEQ ID NO: 223) | (SEQ ID NO: 236) | (SEQ ID NO: 249) |
| | NO: 214) | | | |
| Guide 2 | GCCCCGGCCU | gtgGCCCCGGCCTAA | caccGCCCCGGCC | aaacAGGCAGGTC |
| SpCas9 | AAGACCUGCC | GACCTGCCTAGGact | TAAGACCTGCCT | TTAGGCCGGGGC |
| P347wt | U (SEQ ID | (SEQ ID NO: 224) | (SEQ ID NO: 237) | (SEQ ID NQ: 250) |
| | NO: 215) | | | |
| Guide 3 | CCUAGGCAGG | agtCCTAGGCAGGTC | caccgCCTAGGCAG | aaacCGGCCTAAG |
| SpCas9 | UCUUAGGCCG | TTAGGCCGGGGcca | GTCTTAGGCCG | ACCTGCCTAGGc |
| P347wt | (SEQ ID | (SEQ ID NO: 225) | (SEQ ID NO: 238) | (SEQ ID NO: 251) |
| | NO: 216) | | | |
| Guide 1 | GACGAGCCAG | ggaGACGAGCCAGG | caccGACGAGCCA | caacAGGCCGGGG |
| Nme2Cas9 | GUGGCCCCG | TGGCCCCGGCCTAA | GGTGGCCCCGGC | CCACCTGGCTCG |
| P347WT | GCCU (SEQ ID | GACCtgc (SEQ ID | CT (SEQ ID | TC (SEQ ID |
| | NO: 217) | NO: 226) | NO: 239) | NO: 252) |
| Guide 2 | GUCCUAGGCA | agaGTCCTAGGCAG | caccGT CCT AGGC | caacCCCGGCCTA |
| Nme2Cas9 | GGUCUUAGGC | GTCTTAGGCCGGGG | AGGTCTTAGGCC | AGACCTGCCTAG |
| P347WT | CGGG (SEQ ID | CCACCtgg (SEQ ID | GGG (SEQ ID | GAC (SEQ ID |
| | NO: 218) | NO: 227) | NO: 240) | NO: 253) |
| Guide 1 | GACGAGCCAG | ggaGACGAGCCAGG | caccGACGAGCCA | caacAGGCCAGGG |
| Nme2Cas9 | GUGGCCCUG | TGGCCCTGGCCTAA | GGTGGCCCTGGC | CCACCTGGCTCG |
| P347L | GCCU (SEQ ID | GACCtgc (SEQ ID | CT (SEQ ID | TC (SEQ ID |
| | NO: 9) | NO: 228) | NO: 241) | NO: 254) |
| Guide 2 | GUCCUAGGCA | agaGTCCTAGGCAG | caccGTCCTAGGC | caacCCTGGCCTA |
| Nme2Cas9 | GGUCUUAGGC | GTCTTAGGCCAGGG | AGGTCTTAGGCC | AGACCTGCCTAG |
| P347L | CAGG (SEQ ID | CCACCtgg (SEQ ID | AGG (SEQ ID | GAC (SEQ ID |
| | NO: 10) | NO: 229) | NO: 242) | NO: 255) |
| Guide 3 | UUAGGCCaGG | gtcTTAGGCCaGGGC | caccgTT AGGCCaG | caacGAGCCAGGT |
| Nme2Cas9 | GCCACCUGGC | CACCTGGCTCGTCT | GGCCACCTGGCT | GGCCCtGGCCTAA |
| P347L | UC (SEQ ID | CCgtc (SEQ ID | C (SEQ ID NO: 243) | c (SEQ ID NO: 256) |
| | NO: 11) | NQ: 230) | | |
| Guide 4 | GCCAGGUGGC | cgaGCCAGGTGGCC | caacGCCAGGT GG | caacTCTTAGGCCa |
| Nme2Cas9 | CCUGGCCUAA | CTGGCCTAAGACCT | CCCtGGCCTAAGA | GGGCCACCTGGC |
| P347L | GA (SEQ ID | GCCtag (SEQ ID | (SEQ ID NO: 244) | (SEQ ID NO: 257) |
| | NO: 12) | NO: 231) | | |
| Guide | CUUCUCCAAU | gccCTTCTCCAATGC | caccgCTTCTCCAA | aaacCACCCGTCG |
| NGG2 | GCGACGGGU | GACGGGTGTGGtac | TGCGACGGGTG | CATTGGAGAAGc |
| SpCas9 | G (SEQ ID | (SEQ ID NO: 232) | (SEQ ID NO: 245) | (SEQ ID NO: 258) |
| RHO | NO: 219) | | | |

*Includes nucleotides flanking target. The PAM sequence is indicated in bold. Nucleotides flanking the target site are indicated in lowercase.

TABLE 6
Cloning oligonucleotides

| | | SEQ ID |
|---|---|---|
| OLIGO NAME | SEQUENCE | NO |
| RHO_gene-F | attaggatccAGAGTCATCCAGCTGGAGCCC | 259 |
| RHO_gene-R | taatctcgagTGGGGTTTTTCCCATTCCCAGG | 260 |
| Mut_P347L-F | CGAGCCAGGTGGCCCtGGCCTAAGACCTGCC | 261 |
| Mut_P347L-R | GGCAGGTCTTAGGCCaGGGCCACCTGGCTCG | 262 |
| RHO_cDNA-F | ttaggatccgccaccATGAATGGCACAGAAGG | 263 |
| | CCC | |
| RHO_cDNA-R | taatgaattcTTAAGGAGCCTATGTGACTTCG | 264 |

7.1.1.2. Cell culture

RPE1 and HEK293T/17 cells were obtained from ATCC. 293T-RHO-P347L and RPE-RHO-P347L cells, constitutively expressing the RHO P347L mutant protein, were generated by transduction of parental HEK293T/17 or RPE1 cells, respectively, using a the lentiRHO-P347L vector expressing a blasticidin resistance marker. Transduced cells were pool-selected with 5 μg/ml of blasticidin (Invivogen) in the case of 293T-RHO-P347L, while RPE-RHO-P347L were pool-selected with 20 μg/ml of blasticidin. HEK293T/17 cell pools constitutively expressing wt RHO were generated as described above by transduction with the lentiRHO-wt lentiviral vector. HEK293T/17 cells and the relative RHO P347L stable pools were cultured in a 37° C. incubator with 5% CO₂ using DMEM supplemented with 10% fetal bovine serum (FBS, Life Technologies), 2 mM L-Glutamine (Life Technologies), 10 U/ml penicillin and 10 mg/ml streptomycin (Life Technologies) and blasticidin as indicated above, when needed. RPE1 and relative RHO P347L stable pools were maintained in DMEM-F12 (Life Technologies) with the same supplementations as above. All cell lines were verified mycoplasma-free (PlasmoTest, Invivogen).

7.1.1.3. Lentiviral Vector Production and Transductions

Lentiviral particles were produced by seeding 4×10⁶ HEK 293T/17 cells into a 10 cm dish. The day after the plates were transfected with 10 μg of the desired transfer vector together with 6.5 μg of the packaging vector and 3.5 μg of a VSV-G encoding vector using the polyethylenimine (PEI) method (Casini et al., 2015, J. Virol. 89, 2966-2971). After an overnight incubation, the medium was replaced with fresh complete DMEM and 48 h later the supernatant containing the viral particles was collected, spun down at 500×g for 5 min and filtered through a 0.45 μm PES filter. The titers (RTU, Reverse Transcriptase Units) of viral vector preparations were determined using the SG-PERT (Product Enhanced Reverse Transcriptase) assay (Vermeire et al., 2012, PloS One 7, e50859). Viral vectors were stored at −80° C. until use.

The day before transduction 1×10⁵ HEK293T/17 or RPE1 cells were plated in a 24-well plate. Cells were then transduced by replacing the culture medium with an amount of viral vector preparation corresponding to 1 RTU and incubating overnight. The medium was then replaced with fresh complete DMEM or DMEM-F12 the next morning. For the generation of cell pools stably expressing the RHO P347L mutant, three days after transduction cells were put under blasticidin selection (5 μg/ml for 293T-RHO-P347L and 20 μg/ml for RPE1-RHO-P347L).

Lentiviral transductions of 293T-RHO-P347L and RPE1-RHO-P347L to evaluate editing of the integrated RHO P347L locus were performed essentially as described before using 0.5 RTU per sample. Three days after transduction cells were put under puromycin selection (1 μg/ml, Invivogen) for the following 6 days before sample collection for evaluation of the editing levels.

7.1.1.4. Transfections

10⁵ HEK293T/17 or 293T-RHO-P347L cells were transfected in 24-well plates with 500 ng of Cas9 coding plasmids and 250 ng of the desired pUC19-sgRNA plasmid using either TransIT-LT1 (Mirus Bio) or Lipofectamine 3000 (Life Technologies), according to manufacturer's instructions. Cells were collected 3 days or 6 days after transfection for downstream analyses.

7.1.1.5. Evaluation of Genomic Indels

Genomic DNA was extracted from cell pellets using the QuickExtract solution (Lucigen) according to manufacturer's instructions. The HOT FIREPol MultiPlex Mix (Solis Biodyne) was used to amplify the endogenous RHO locus using primers ICE-RHO_endo-F and ICE-RHO_endo-R (Table 7) and the RHO P347L cDNA using primers ICE-RHO_cDNA-F and ICE-RHO_cDNA-R (Table 7), specifically detecting the integrated locus. The amplicon pools were Sanger sequenced (Mix2seq kits, Eurofins Genomics) and the indel levels were evaluated using the Synthego ICE webtool (https://ice.synthego.com).

TABLE 7
Oligonucleotides used for PCR amplification and
indel analysis

| OLIGO NAME | SEQUENCE | SEQ ID NO: |
|---|---|---|
| ICE-RHO_endo-F | TCCAGGAAGCTGGATTTGAGTGG | 265 |
| ICE-RHO_endo-R | TATGAGTTGCTGCTGCTGCGAG | 266 |
| ICE-RHO_cDNA-F | ACCCTGGGCGGTGAAATTG | 267 |
| ICE-RHO_cDNA-R | GGCGGAATTCTTAAGGAGCC | 268 |

7.1.1.6. Western Blots

Cells were lysed in RIPA buffer (NaCl 150 mM, NP-40 1%, Sodium deoxycholate 0.5%, SDS 0.1%, Tris 25 mM in ddH2O supplemented with 1% of Halt protease inhibitor cocktail (Thermo Fisher Scientific)). Samples were not boiled before loading on the gel. Cell extracts were separated by SDS-PAGE using the PageRuler Plus Protein Standards as the standard molecular mass markers (Thermo Fisher Scientific). After electrophoresis, samples were transferred to 0.45 μm nitrocellulose membranes (GE Healthcare). The membranes were incubated with mouse anti-Rhodopsin clone 4D2 antibody (MABN15, Sigma-Aldrich, dilution 1:1,000) and mouse anti-GAPDH 6C5 (sc-32233 Santa Cruz Biotechnology, dilution 1:4,000) and with the HRP conjugated mouse IgGκ binding protein (sc-516102, Santa Cruz Biotechnology, 1:5,000) for ECL detection. Images were acquired and bands were quantified using the UVItec Alliance detection system.

7.1.2. Results

7.1.2.1. Design and Validation of a Genome Editing Strategy to Target the RHO P347 Locus

In order to design guide RNAs suitable to target the RHO P347 locus, the genomic region neighboring the residue of interest, which is located immediately before the last amino acid of the Rhodopsin protein, was inspected to identify PAM sequences for Cas9 endonucleases. Given the aim of designing an allele-specific genome editing strategy to specifically knock-out the mutated RHO P347L allele, a mandatory requirement for the selected gRNAs was that their target sequence must include the mutation giving rise to the P to L substitution in position 347 of the Rhodopsin protein.

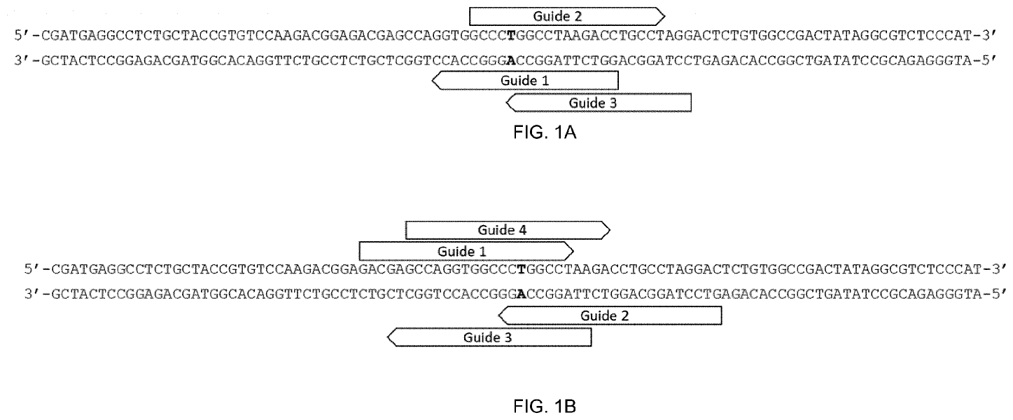

Three different guide RNAs for SpCas9 (NGG PAM, reported in Table 5, represented in FIG. 1A) were identified as well as four suitable guide RNAs for the small Cas9 ortholog Nme2Cas9 (Edraki et al., 2019, Mol. Cell 73, 714-726.e4) (NNNNCC PAM, reported in Table 5, represented in FIG. 1B). All these guides span the RHO P347L mutation, which was located at various positions along the spacer length, according to the localization of the respective PAMs on the target genomic locus.

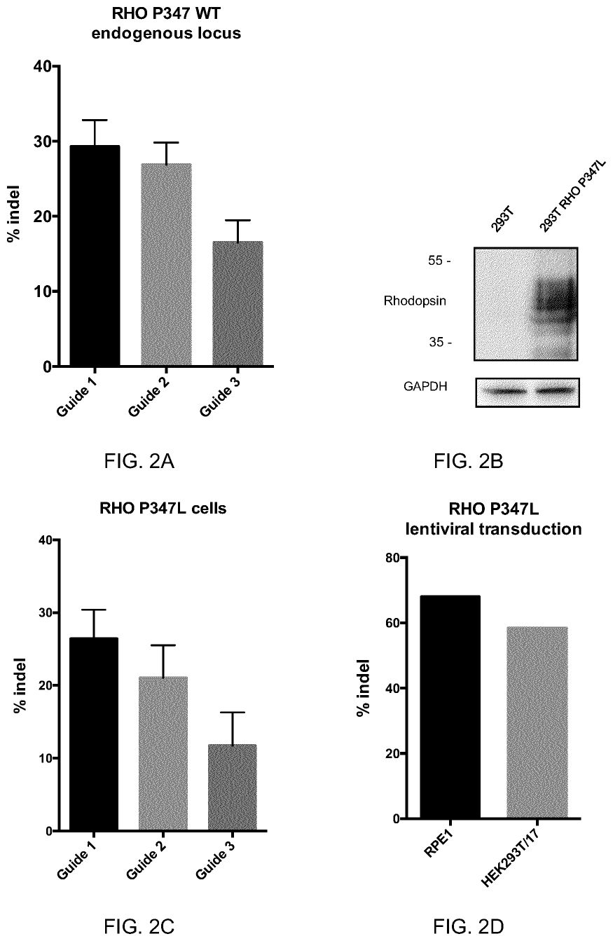

Next, the editing efficacy of the three SpCas9 sgRNA as well as two Nme2Cas9 sgRNAs (Guide 1 and Guide 2) was preliminarily evaluated by targeting the endogenous RHO P347 wt locus in HEK293T/17 cells. For these studies, a version of each guide perfectly matching the wt locus (and thus not containing the single nucleotide substitution leading to the P347L mutation) was exploited. As shown in FIG. 2A and FIG. 5, after transient transfection of HEK293T/17 cells with each nuclease together with the respective guides, indels levels ranging between 10% and 30% with the SpCas9 sgRNA being generally more efficient (15-30%) than those for Nme2Cas9 (10-20%).

7.1.2.2. Evaluation of RHO P347L Targeting

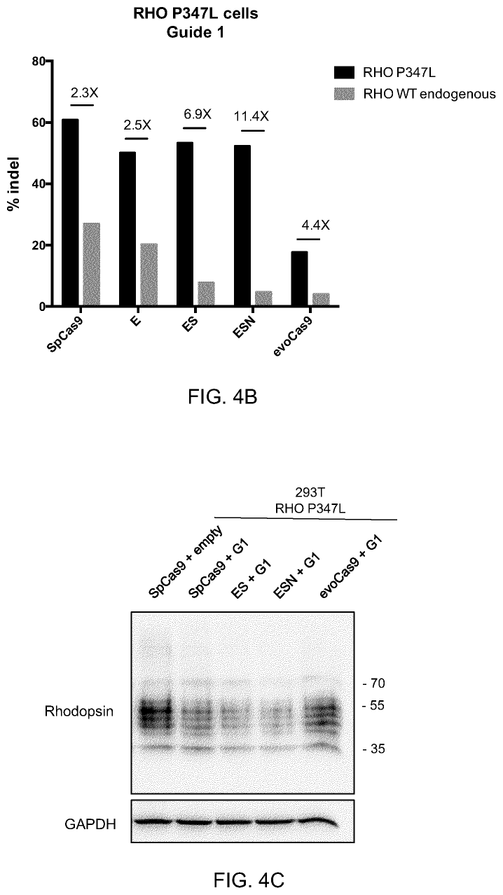

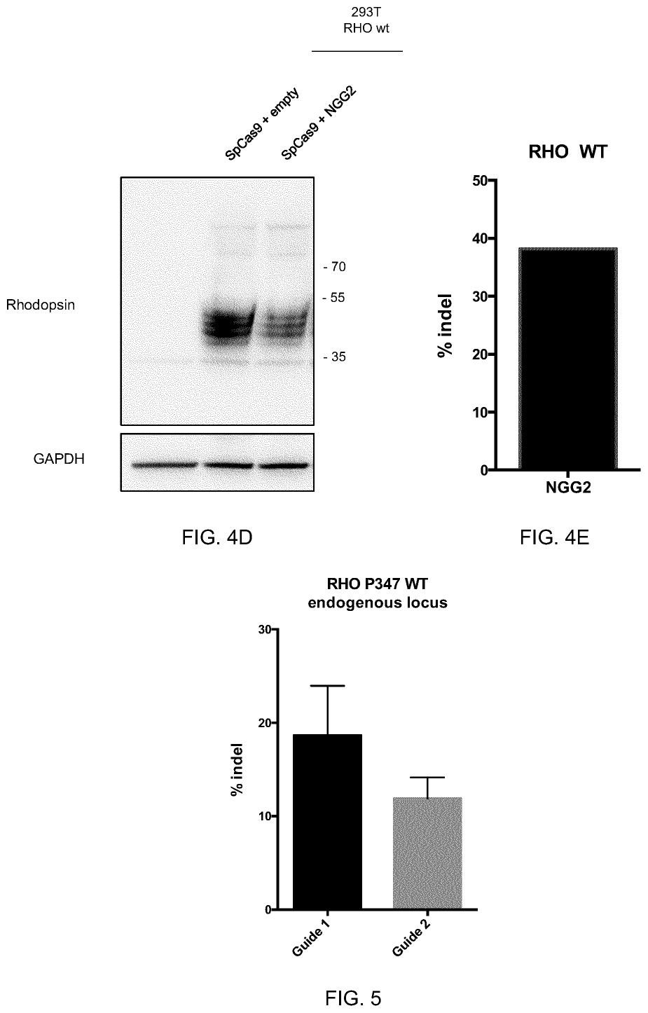

To specifically measure indel formation induced by the selected nuclease-sgRNA couples on the mutated RHO P347L locus a cell pool stably expressing the RHO P347L mutant protein was generated by lentiviral transduction of parental HEK293T/17 cells with lentiRHO-P347L. A control cell line was generated by transduction of HEK293T/17 cells with a lentiviral vector expressing the wt RHO cDNA (lentiRHO-wt). These two cell lines were constitutively expressing the RHO protein, which is normally present only in photoreceptor cells. The correct expression of both the wt RHO and the P347L mutant was verified by western blotting (FIG. 2B and FIG. 4C, left panel), using an antibody specifically recognizing the N-terminus of the protein to avoid any interference of the P347L mutation with epitope recognition. In both cases Rhodopsin presented as a series of bands comprised between 45-55 kDa (FIGS. 2B and 4C).

Given the higher efficacy, only SpCas9 guide RNAs were initially validated in RHO P347L-expressing cells. The 293T-RHO-P347L cell pool was transiently transfected with wt SpCas9 and the three different sgRNAs designed against the RHO P347L locus (Guide1-Guide2-Guide3, see Table 5). FIG. 2C shows that the levels of measured indels are in accordance with those previously obtained on the endogenous wt RHO locus (10-25%), with Guide1 being the most efficient in both study settings (compare: 30% on RHO wt, FIG. 2A, and 25% on RHO P347L, FIG. 2C).

As a further confirmation, a pool of RPE1 cells stably expressing the RHO P347L mutant protein (RPE-RHO-P347L) was generated and the level of indel formation at the integrated RHO P347L locus was measured after transduction with a lentiviral vector expressing wt SpCas9 and the best performing sgRNA Guide 1. As a reference, indel formation at the RHO P347L locus was measured also in 293T-RHO-P347L cells transduced with the same lentiviral vector, demonstrating similar levels of gene editing (FIG. 2D).

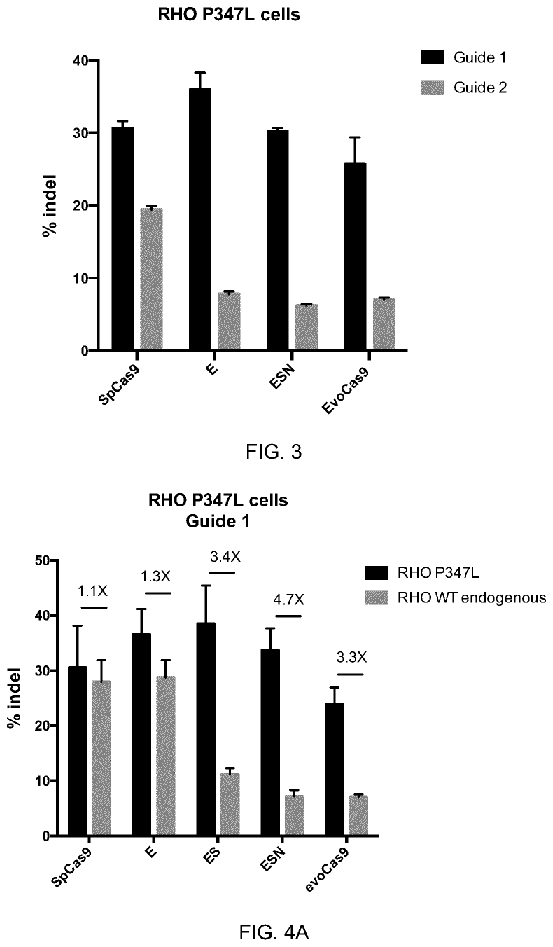

Since this gene editing approach was designed to exploit high-fidelity SpCas9 variants to induce an allele-specific downregulation of the mutated P347L allele, the efficacy of the two best performing sgRNAs (Guide1 and Guide2) was evaluated in combination with the E (K526E mutation), ESN (K526E+R661S+Y515N mutations) and evoCas9 (M495V+Y515N+K526E+R661Q mutations) (Casini et al., 2018, Nat. Biotechnol. 36: 265-271) high-fidelity SpCas9 variants in parallel with wt SpCas9 by transient transfection of 293T-RHO-P347L cells. While Guide 1 was able to induce consistent levels of editing with all the tested variants (editing levels approx. 25-30%, FIG. 3), Guide 2 produced appreciable indel formation only with wt SpCas9 (approx. 20% editing, FIG. 3).

7.1.2.3. Allele-Specific Knock-Out of the RHO P347L Mutant

The allele-specificity of SpCas9 Guide 1, the most efficient sgRNA validated so far, was evaluated by measuring indel formation in 293T-RHO-P347L stable cell pools both at the integrated RHO P347L locus, the on-target, and at the endogenous RHO wt locus, which should be preserved and can thus be considered an off-target site, differing from the intended target by a single nucleotide mismatch (the P347L mutation).

The allele specificity of the editing strategy was thus evaluated by transiently transfecting 293T-RHO-P347L cells with Guide 1 together with a panel of high-fidelity SpCas9 variants (E containing the mutation K526E; ES containing the mutation K526E+R661S; ESN containing the mutation K526E+R661S+Y515N; evoCas9 containing the mutations M495V, Y515N, K526E, and R661Q and wt SpCas9 as a reference. The on-target editing on the integrated RHO P347L locus was similar for all the tested variants (30-40%), except for evoCas9 for which a slight decrease in editing was measured (FIG. 4A, black bars). The allele specificity was then compared among the different SpCas9 variants by evaluating indel formation at the endogenous RHO wt locus. While the ES, ESN and evoCas9 mutants showed significantly decreased levels of cleavage of the wt locus, in this study wt SpCas9 and the E mutant were not able to discriminate between the two sequences to a large degree, leading to similar levels of editing both on the wt and P347L locus (FIG. 4A, grey bars). In addition, the contribution of the most frequent indels to gene editing was analyzed using the ICE webtool (Table 8 reports data for a representative study) showing three major types of indel (+1, −1, −2 and −8) generated by most of the tested variants. Interestingly, evoCas9 showed a slightly different indel profile with minor contributions from −1 and −2 edits while demonstrating a unique −8 deletion. For all the tested variants, including wt SpCas9, the +1 insertion was the most common repair outcome after cleavage.

TABLE 8
Indels profile for P347 Locus

| | 1 | −1 | −2 | −8 |
|---|---|---|---|---|
| SpCas9 | 26% | 7% | 5% | 1% |
| E | 31% | 6% | 3% | 1% |
| ES | 32% | 6% | 5% | 3% |
| ESN | 24% | 6% | 4% | 4% |
| evoCas9 | 16% | 1% | — | 7% |

These data were further confirmed by repeating the study using a different transfection protocol which substituted the Mirus TransIT-LT1 transfection reagent with Lipofectamine 3000 from Life Technologies. In this case (FIG. 4B, black bars), increased editing levels were observed with all the tested variants (up to 50-60% indel formation) with the exception of evoCas9 (approx. 20% indel formation). On the other hand, off-target indel levels were very similar to those measured in previous studies, possibly suggesting that an editing plateau had been already reached when using TransIT-LT1 as the transfection reagent (FIG. 4B, grey bars). For both sets of studies the on-/off-target ratio obtained with each tested SpCas9 variants was calculated (FIG. 4A-B), showing, as expected, increased ratios with the ES, ESN and evoCas9 mutants compared to wt SpCas9 and E mutant, indicating an increased preference to cleave the target RHO P347L locus over the wild-type counterpart.

Given the availability of cells constitutively expressing the RHO P347L mutant, intracellular RHO P347L protein levels were evaluated after treatment with Guide 1 in combination with the panel of high-specificity SpCas9 variants (ES, ESN, evoCas9) as well as wt SpCas9. As expected, and in accordance with the editing data, a clear decrease in the intracellular levels of RHO P347L was observed for all the tested variants except for evoCas9, for which the decrease was less pronounced (FIG. 4C). As a comparison, the decrease of the intracellular levels of wt RHO in 293T-RHO-wt cells, stably expressing the wt RHO protein, was evaluated after transient transfection with wt SpCas9 and a sgRNA (NGG2, Table 5) targeting a different region of the RHO coding sequence located at the N-terminus of the protein. The overall indel formation was in line with those observed for the P347L locus (approx. 40%, FIG. 4E). In addition, in accordance with the measured indel formation at this locus, a decrease in Rhodopsin levels was observed (FIG. 4D).

### 7.2. Example 2: SpCas9 Editing of RHO P347L Gene

7.2.1. Materials and Methods

7.2.1.1. Plasmids and Oligonucleotides

SpCas9 and high-fidelity variants ES and EQ containing the K526E+R661S/Q mutations, ESN and EQN containing the K526E+R661S/Q+Y515N mutations, and evoCas9 containing the K526E+R661Q+Y515N+M495V mutations were expressed from plasmids containing a CBh-driven expression cassette. All sgRNAs were expressed from a pUC19 plasmid containing U6-driven Pol III expression cassettes (pUC19-sgRNA). The spacer sequences of the sgRNAs and the oligonucleotides used to generate the expression constructs are reported in Table 5. Spacers were cloned as annealed oligonucleotides into a double Bbsl site of the pUC19-sgRNA plasmids containing the corresponding constant sgRNA region for SpCas9 (SEQ.ID.) (3′ sgRNA sequence 8 as set forth in Table 3).

The human rhodopsin (RHO) gene (including its entire 5′- and 3′-UTRs) was PCR-amplified using the primers RHO_gene-F and RHO_gene-R from genomic DNA extracted from HEK293T/17 cells and cloned into a plasmid containing a CMV-driven expression cassette, generating the pCMV-RHO-wt plasmid. The RHO P347L mutation was introduced in this construct by site-directed mutagenesis using primers Mut_P347L-F and Mut_P347L-R, reported in Table 9, generating the pCMV-RHO-P347L plasmid. Additionally, a fragment of the RHO P347L gene was amplified from pCMV-RHO-P347L using primers RHO_minigene-F and RHO_minigene-R and cloned into plasmid containing a CMV-TetO promoter, whose activity can be regulated by the addition of doxycycline to the cell culture medium when the tetracycline repressor (TetR) is expressed in target cells. The oligonucleotides exploited for cloning are reported in Table 9.

TABLE 9
Cloning oligonucleotides

| | | SEQ |
|---|---|---|
| | | ID |
| OLIGO NAME | SEQUENCE | NO |
| RHO_gene-F | attaggatccAGAGTCATCCAGCTGGAGCCC | 259 |
| RHO_gene-R | taatctcgagTGGGGTTTTTCCCATTCCCAGG | 260 |
| Mut_P347L-F | CGAGCCAGGTGGCCCtGGCCTAAGACCTGCC | 261 |
| Mut_P347L-R | GGCAGGTCTTAGGCCaGGGCCACCTGGCTCG | 262 |
| RHO_cDNA-F | ttaggatccgccaccATGAATGGCACAGAAGG | 263 |
| | CCC | |
| RHO_cDNA-R | taatgaattcTTAAGGAGCCTATGTGACTTCG | 264 |
| RHO_minigene- | ttaggatccgccaccATGAATGGCACAGAAGG | 263 |
| F | CCC | |
| RHO_minigene- | taattctagaGGAGCCTCATTAATTATTTCTT | 269 |
| R | AA | |

A lentiviral vector expressing TetR and a blasticidin resistance marker (lenti-TetR-blast) was used to produce viral particles to generate TetR-expressing HEK293 cells.

The cDNA for RHO P347L was obtained by reverse transcription using the Revertaid RT Reverse Transcription Kit (ThermoFisher Scientific) of total RNA extracted from HEK293T/17 cells transfected with pCMV-RHO-P347L and was amplified using the primers RHO_cDNA-F and RHO_cDNA-R (Table 9) for cloning into a lentiviral vector for the production of stable cell lines, generating lentiRHO-P347L. The construct included also part of the 3′-UTR sequence of the RHO gene. In addition, the vector contained a blasticidin selection marker. All PCR products used for preparing plasmid constructs were generated using the Phusion high-fidelity DNA polymerase (ThermoFisher Scientific). All oligonucleotides were obtained from Eurofins Genomics. Constructs were verified by Sanger sequencing (Eurofins Genomics).

7.2.1.2. Cell Culture

HEK293T/17 and HEK293 cells were obtained from ATCC. 293T-RHO-P347L cells, constitutively expressing the RHO P347L mutant protein, were generated by transduction of parental HEK293T/17 using the lentiRHO-P347L vector expressing a blasticidin resistance marker. Transduced cells were pool-selected with 5 μg/ml of blasticidin (Invivogen). To obtain HEK293T/17 cell clones having integrated a definite number of RHO P347L copies, a limiting dilution of the cell pools previously generated was performed.

HEK293 cells expressing the TetR protein were generated by lentiviral transduction using lenti-TetR-blast and pool-selected with 5 μg/ml of blasticidin. Subsequently, the pool was stably transfected with pCMV-TO-RHO-P347L by pool-selection with 500 μg/ml of G418 (Invivogen), generating 293TetO-RHO-P347L cells.

HEK293 and HEK293T/17 cells, the relative RHO P347L stable pools and clones were cultured in a 37° C. incubator with 5% CO₂ using DMEM supplemented with 10% fetal bovine serum (FBS, Life Technologies), 2 mM L-Glutamine (Life Technologies), 10 U/ml penicillin and 10 mg/ml streptomycin (Life Technologies) and blasticidin or neomycin as indicated above, when needed. All cell lines were verified mycoplasma-free (PlasmoTest, Invivogen).

7.2.1.3. Lentiviral Vector Production and Transductions

Lentiviral particles were produced by seeding 4×10⁶ HEK 293T/17 cells into a 10 cm dish. The day after the plates were transfected with 10 μg of the desired transfer vector together with 6.5 μg of the packaging vector and 3.5 μg of a VSV-G encoding vector using the polyethylenimine (PEI) method (Casini et al., 2015, J. Virol. 89:2966-2971). After an overnight incubation, the medium was replaced with fresh complete DMEM and 48 h later the supernatant containing the viral particles was collected, spun down at 500×g for 5 min and filtered through a 0.45 μm PES filter. The titers (RTU, Reverse Transcriptase Units) of viral vector preparations were determined using the SG-PERT (Product Enhanced Reverse Transcriptase) assay (Vermeire et al., 2012, PloS One 7: e50859). Viral vectors were stored at −80° C. until use.

The day before transduction 1×10⁵ HEK293T/17 or HEK293 cells were plated in a 24-well plate. Cells were then transduced by replacing the culture medium with an amount of viral vector preparation corresponding to 1 RTU mixed with fresh medium and incubating overnight. The medium was then replaced with fresh complete DMEM the next morning. For the generation of cell pools stably expressing the RHO P347L mutant or the TetR protein, three days after transduction cells were put under blasticidin selection (5 μg/ml).

7.2.1.4. Transfections

10⁵ 293 T-RHO-P347L or 293TetO-RHO-P347L cells (plated the day before transfection) were transfected in 24-well plates with 500 ng of Cas9 coding plasmids and 250 ng of the desired pUC19-sgRNA plasmid using either TransIT-LT1 (Mirus Bio) or Lipofectamine 3000 (Life Technologies), according to manufacturer's instructions. Cells were collected 3 days after transfection for indel evaluation. To measure intracellular levels of RHO P347L protein as well as its mRNA levels after editing, after transfection cells were kept in culture for 7 days before collection of cell pellets. During studies using 293TetO-RHO-P347L cells, the medium was changed the day after transfection and doxycycline was added to a final concentration of 100 ng/ml until the end of the studies.

7.2.1.5. Transgene Copy Number

The copy number of integrated RHO P347L genes in each cell clone obtained from the parental stable pool was evaluated by qPCR performed on genomic DNA using the primers reported in Table 10 and the HOT FIREPol EvaGreen qPCR Supermix (Solis Biodyne). Standard curves to measure the input number of cellular genomes and the absolute number of transgene copies were generated using a pUC19 plasmid containing the GAPDH gene portion amplified in the qPCR assay and the transfer vector plasmid used to package the lentiviral vector exploited to transduce 293T/17 cells, respectively. By comparing the absolute number of transgene copies in each sample with the number of input genomes (measured through GAPDH copy number) an estimation of the number of transgenes per genome has been obtained.

TABLE 10
Oligonucleotides used for transgene copy number
quantification by qPCR assays

| OLIGO NAME | SEQUENCE | SEQ ID NO |
|---|---|---|
| GAPDH-CN-F | CACAGTCCAGTCCTGGGAAC | 270 |
| GAPDH-CN-R | TAGTAGCCGGGCCCTACTTT | 271 |
| RHO-plasmid-CN-F | GTCACCGTCCAGCACAAGA | 272 |
| RHO-plasmid-CN-R | GGCAATTTCACCGCCCAG | 273 |

7.2.1.6. Evaluation of Genome Editing

Genomic DNA was extracted from cell pellets using the QuickExtract solution (Lucigen) according to manufacturer's instructions. The HOT FIREPol MultiPlex Mix (Solis Biodyne) was used to amplify the endogenous RHO locus using primers ICE-RHO_endo2-F and ICE-RHO_endo2-R (reported in Table 11) and the RHO P347L cDNA using primers ICE-RHO_cDNA-F and ICE-RHO_cDNA-R (reported in Table 11), specifically detecting the integrated locus in 293T-RHO-P347L cells. To detect indel formation at Guide 1 off-target sites identified by GUIDE-seq the primers ICE-RHO-OFF1/3-F/R reported in Table 11 have been used. The amplicon pools were Sanger sequenced (Microsynth) and the indel levels were evaluated using the Synthego ICE webtool (ice.synthego.com). For studies in 293TetO-RHO-P347L cells, indel formation was measured as described before using primers ICE-RHO_endo2-F and ICE-RHO_plasmid-R (Table 11) to amplify the integrated RHO P347L locus, and the primer ICE-RHO_endo-R to Sanger sequence the amplicons for ICE analysis.

TABLE 11
Oligonucleotides used for PCR amplification and
indel analysis

| OLIGO NAME | SEQUENCE | SEQ ID NO |
|---|---|---|
| ICE-RHO_endo2-F | ACCTCCGAGGGGTAAACAGT | 274 |
| ICE-RHO_endo2-R | TTAAGGAGCCTATGTGACTTCG | 275 |
| ICE-RHO_cDNA-F | ACCCTGGGCGGTGAAATTG | 267 |
| ICE-RHO_cDNA-R | GGCGGAATTCTTAAGGAGCC | 268 |
| ICE-RHO-OFF1-F | AAGCGACTTGCTCCCCTAAC | 276 |
| ICE-RHO-OFF1-R | GACCACCTGGTCCACTATGC | 277 |
| ICE-RHO-OFF2-F | GTGAGGGGTGTAACAGAACA | 278 |
| ICE-RHO-OFF2-R | TTGCACCTCTGACTGGTTGG | 279 |
| ICE-RHO-OFF3-F | GATGGTTCAGGAAGACGGGT | 280 |
| ICE-RHO-OFF3-R | CGGGTGATGTGAGCAACTCA | 281 |
| ICE-RHO-plasmid-R | CAGCGAGCTCTAGCATTTAGG | 282 |

7.2.1.7. Evaluation of RHO P347L mRNA Levels

Total RNA was extracted from cell pellets using the NucleoZOL reagent (Macherey-Nagel) according to the manufacturer's instructions. Total RNA was then retrotranscribed using the Revertaid RT Reverse Transcription Kit (ThermoFisher Scientific) and random hexamer primers. The relative quantification of RHO P347L mRNA expression level was obtained by qPCR using primers reported in Table 12 and the HOT FIREPol EvaGreen qPCR Supermix (Solis Biodyne). The relative RHO P347L mRNA expression levels were normalized to the expression level of the GAPDH housekeeping gene, using ΔΔCt quantification method.

TABLE 12
Oligonucleotides used for RT-qPCR

| OLIGO NAME | SEQUENCE | SEQ ID NO |
|---|---|---|
| qPCR-GAPDH-F | TCGGAGTCAACGGATTTGGT | 283 |
| qPCR-GAPDH-R | TCGCCCCACTTGATTTTGGA | 284 |
| qPCR-RHO-F | GGTCCAGGTACATCCCCGAG | 285 |
| qPCR-RHO-R | GTGAAGACGAGCTGCCCATA | 286 |

7.2.1.8. Western Blots

Cells were lysed in RIPA buffer (NaCl 150 mM, NP-40 1%, Sodium deoxycholate 0.5%, SDS 0.1%, Tris 25 mM in ddH2O supplemented with 1% of Halt protease inhibitor cocktail (Thermo Fisher Scientific)). Samples were not boiled before loading on the gel. Cell extracts were separated by SDS-PAGE using the PageRuler Plus Protein Standards as the standard molecular mass markers (Thermo Fisher Scientific). After electrophoresis, samples were transferred to 0.45 μm nitrocellulose membranes (GE Healthcare). The membranes were incubated with mouse anti-Rhodopsin clone 4D2 antibody (MABN15, Sigma-Aldrich, dilution 1:1,000) and mouse anti-GAPDH 6C5 (sc-32233 Santa Cruz Biotechnology, dilution 1:4,000) and with the HRP conjugated mouse IgGκ binding protein (sc-516102, Santa Cruz Biotechnology, 1:5,000) for ECL detection. Images were acquired and bands were quantified using the UVltec Alliance detection system.

7.2.1.9. Genome-Wide Evaluation of Off-Target Sites

2×10⁵ 293 T-RHO-P347L (clone 4) cells were transfected with 750 ng of each Cas9-expressing plasmid, together with 250 ng of each sgRNA-coding plasmid or an empty pUC19 plasmid, 10 pmol of the bait dsODN containing phosphorothioate bonds at both ends designed according to the original GUIDE-seq protocol (Tsai et al., 2015, Nat. Biotechnol. 33:187-197) and 50 ng of a pEGFP-IRES-Puro plasmid, expressing both EGFP and the puromycin resistance gene. The day after transfection cells were detached and selected with 1 μg/ml of puromycin for 48 h to eliminate non-transfected cells. Cells were then collected and genomic DNA was extracted using the Nucleospin Tissue kit (Macherey-Nagel) following the manufacturer's instructions and sheared to an average length of 500 bp with the Covaris M220 sonicator device according to manufacturer's indications. Library preparations were performed with the original adapters and primers according to previous work (Tsai et al., 2015, Nat. Biotechnol. 33:187-197). Libraries were sequenced using the Illumina MiSeq sequencing system using the Miseq Reagent kit V2-300 cycles (2×150 bp paired-end). Raw sequencing data (FASTQ files) were analyzed using the GUIDE-seq computational pipeline (Tsai et al., 2016, Nat. Biotechnol. 34:483).

7.2.2. Results

7.2.2.1. Evaluation of RHO P347L Knockout in Different Cell Models

The targeting strategy designed to knock-out RHO P347L while preserving the wt allele of the gene is based on the generation of a single cut at the level of the mutation using a selected sgRNA (Guide 1) in combination with high-fidelity SpCas9 variants previously described in literature (Casini et al., 2018, Nat. Biotechnol. 36:265-271). This strategy has been presented in Example 1.

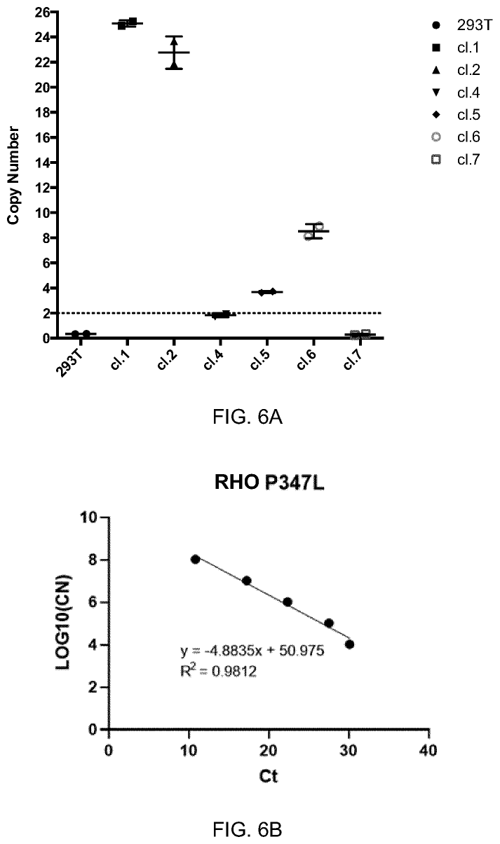

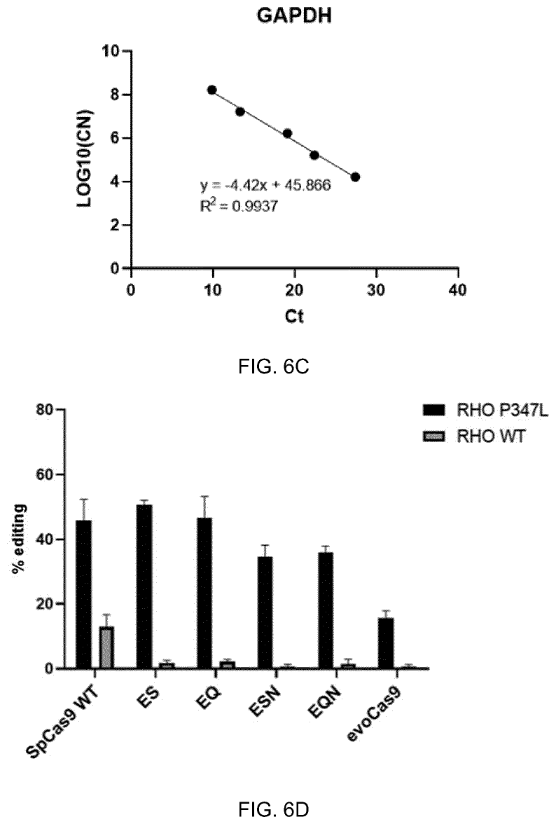

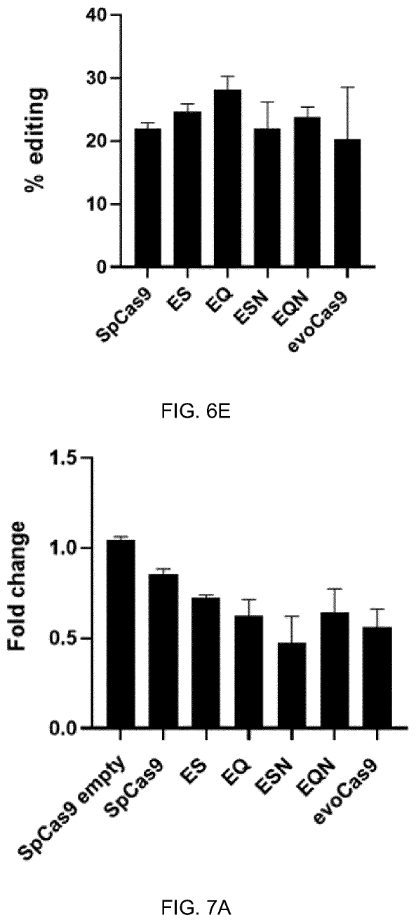

Given the absence of commonly available immortalized cell lines expressing rhodopsin, in order to evaluate the formation of indels at the RHO P347L locus and the consequent effects on the expression of the mutated RHO mRNA and protein, different cell line models expressing the mutant protein of interest were generated. As a first model, HEK293T cells were transduced with a lentiviral vector expressing RHO P347L cDNA under the control of a SFFV promoter to generate stable pools. Clones were then isolated from the pool and the copy number of the transgene was evaluated using qPCR (FIG. 6A) and standard curves (FIG. 6B-C) to calculate the absolute number of integrations per genome in the different isolated cell lines. A second model is represented by HEK293 cells expressing the tetracycline repressor (TetR) that were stably transfected with a tetracycline-regulated RHO P347L transgene preserving the intron-exon structure of the original RHO gene to obtain a cell pool having inducible transgene expression to avoid possible toxicities due to the continuous presence of high intracellular levels of mutant RHO. Of note, both constructs included part of RHO 3′-UTR.

Among the different clones isolated for the 293T-RHO-P347L model, a line having two integrated copies of the transgene (see FIG. 6A, clone 4) was selected for further studies. Absolute copy number was evaluated using standard curves generated for the target RHO P347L transgene (FIG. 6B) and a housekeeping gene with known copy number (GAPDH, FIG. 6C). Indel formation at the RHO P347L integrated locus was evaluated after transient transfection of 293T-RHO-P347L clone 4 with Guide 1 and the different high-fidelity SpCas9 variants or wt SpCas9. As shown in FIG. 6D, the ES (K526E+R661S) and EQ (K526E+R661Q) variants showed editing levels that were comparable to wt SpCas9 (40-50% indel). The ESN (Y515N+K526E+R661S) and EQN (Y515N+K526E+R661Q) mutants were slightly less active (35-40% indel), while evoCas9 produced less than half of the editing events observed with wt SpCas9 (15-20% indel). Additionally, the allele specificity of each variant in combination with Guide 1 was evaluated by measuring indel formation at the endogenous wt RHO locus. While wt SpCas9 produced appreciable levels of cleavage (approximately 10-15% of editing), none of the tested high-fidelity variants generated enough cleavages to go above the sensitivity of the assay used to measure indel formation (FIG. 6D).

Genome editing was also evaluated at the integrated mutant RHO locus in the 293TetO-RHO-P347L stable cell pool using similar study conditions. As for 293T-RHO-P347L clone 4, the results shown in FIG. 6E demonstrate that most of the tested SpCas9 high-fidelity variants maintain editing levels comparable to wt SpCas9. The lower editing levels observed in this cell system may be due to the fact that cells were pool selected and thus RHO P347L copy number may be high and variable among different cells in the population.

Overall the data demonstrate that Guide 1 in combination with high-fidelity SpCas9 variants is able to specifically induce indel formation on the RHO P347L allele while sparing the wt counterpart, thus validating the targeting approach selected for RHO P347L. In addition, indel formation was confirmed in two independent cellular models generated to express the P347L RHO mutant. Notably, among the variants, both the double ES and EQ mutants showed cleavage activity similar to wt SpCas9 while showing superior allele specificity.

7.2.2.2. Functional Validation of RHO P347L Knockout

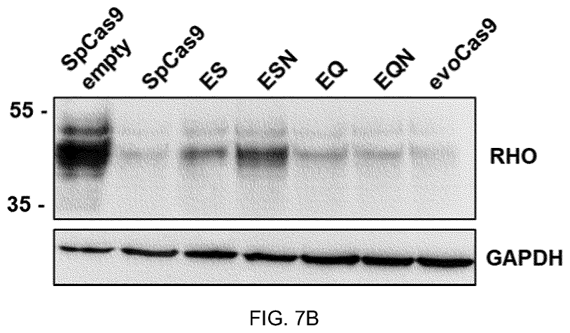

As a first step to confirm that the introduction of indels at the level of RHO P347L mutation is indeed able to downregulate mutant protein expression, RHO P347L mRNA levels were evaluated in 293T-RHO-P347L clone 4. After transient transfection with Guide 1 together with the panel of high-fidelity SpCas9 variants or with wt SpCas9, cells were kept in culture for 7 days and then collected for genomic DNA and total RNA extraction. A qPCR assay was exploited to measure mRNA levels produced from the integrated mutant RHO transgene. As shown in FIG. 7A, decreases up to 50% of RHO P347L mRNA were observed after treatment with the different SpCas9 variants in combination with Guide 1, confirming that a single cut at the level of the P347L mutation can promote mRNA destabilization and degradation. Of note, all the high-fidelity SpCas9 variants were able to induce measurable mRNA downregulation.

To further confirm these results, the intracellular levels of the RHO P347L protein were evaluated in the 293TetO-RHO-P347L cell pool 7 days after transient transfection with Guide 1 together with wt SpCas9 or the different high-fidelity variants. Notably, a strong downregulation of the levels of the mutant protein was observed with all the tested SpCas9 (FIG. 7B).

Taken together, these data indicate that targeting the RHO P347L allele produces downregulation of the mutant protein as well as of its mRNA and it is thus reasonable to expect that such elimination of mutant RHO may arrest photoreceptor death thus blocking the disease phenotype when such targeting strategy is deployed in the retina of an affected patient.

7.2.2.3. Evaluation of the Genome-Wide Specificity of the Targeting Strategy

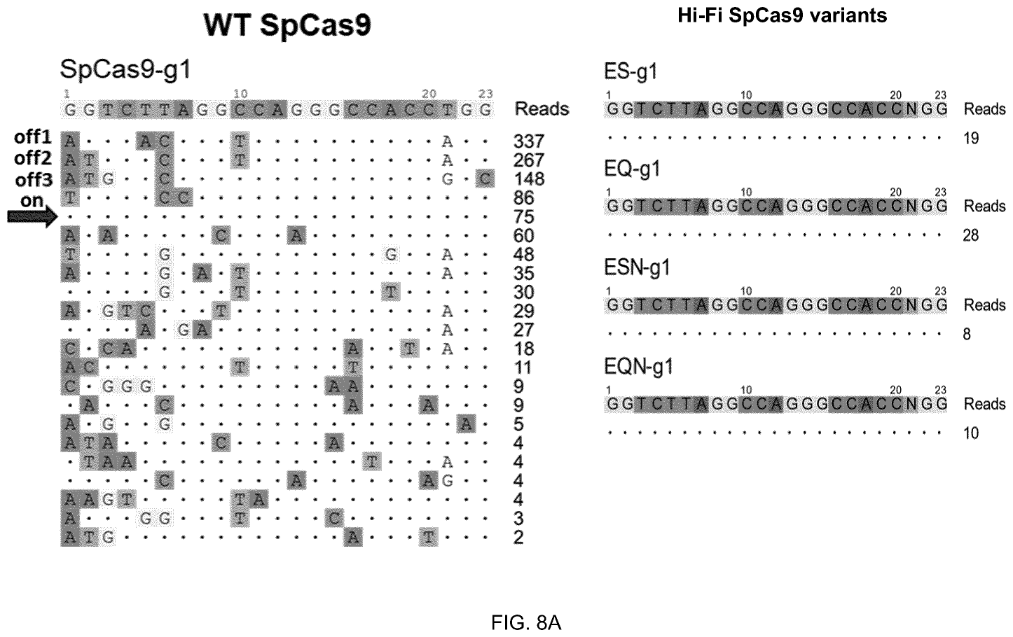

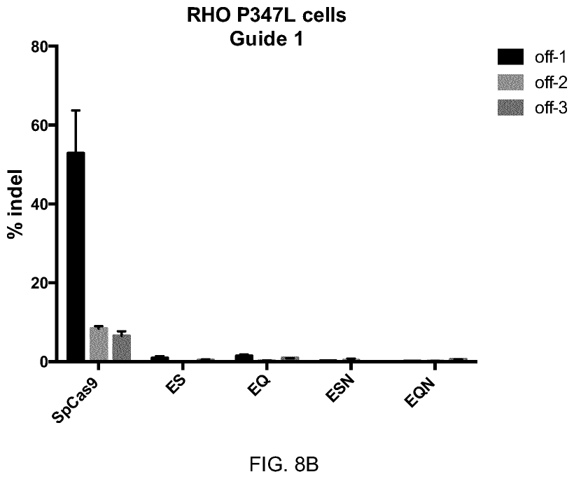

Having established the knock-out efficacy of the targeting strategy, in view of its possible translation into the clinic, it is of primary importance to evaluate the targeting specificity of Guide 1 in combination with the different SpCas9 variants evaluated in the present example to exclude possible unwanted cleavages in the target cell genome. To this aim, a comprehensive characterization of the genome-wide off-targeting profile of Guide 1 was conducted using the GUIDE-seq protocol, that relies on the integration of a double-stranded oligonucleotide into double-strand breaks generated by the nuclease in order to tag the genomic locus for downstream identification through next-generation sequencing. As shown in FIG. 8A, GUIDE-seq studies conducted in 293T-RHO-P347L clone 4 cells demonstrated that while numerous off-targets events could be detected for wt SpCas9, no off-target sites were captured with any of the high-fidelity variants tested (ES, EQ, ESN, EQN). The RHO P347L on-target site was detected in all samples, demonstrating the successful execution of the protocol.

To further confirm that the sites identified through GUIDE-seq were bona fide off-targets, the indel formation at the top three detected loci was measured after transfection of 293T-RHO-P347L clone 4 cells with Guide 1 together with the panel of previously tested SpCas9 high-fidelity variants. While robust cleavage was observed for all three off-targets when Guide 1 was used in combination with wt SpCas9, no indel formation was detected (within the sensitivity limits of the assay) with any of the high-fidelity variants tested, thus confirming the absence of off-targets as determined by GUIDE-seq (FIG. 8B).

Overall the data demonstrate that the combination of Guide 1 with high-fidelity SpCas9 variants has a safe genome-wide profile as no additional cleaved sites could be detected beside the intended target. Of note, this is a clear demonstration of the utility of high-fidelity Cas9 variants to perform high-precision genome editing in all cases in which the selection of the sgRNA target is constrained by study needs, thus expanding the range of guides which can be safely used to target specific loci into the cellular genome.

### 7.3. Example 3: Double-sgRNA Targeting Strategy

7.3.1. Materials and Methods

The Material and Methods of the present Example are shared with those of Example 2. Additional Material and Methods specific for the present Example are reported here below.

7.3.1.1. Plasmids and Oligonucleotides

The plasmids used to express SpCas9 variants and guide RNAs have been described in the Methods section of Example 2. Additional sgRNA spacers used in the present Example to target RHO intron 4 are reported in Table 13.

TABLE 13
sgRNA spacers, target sequences and cloning oligonucleotides

| Guide | spacer | genomic sequence* | cloning oligo 1^{§} | cloning oligo 2^{§} |
|---|---|---|---|---|
| sg- | GCCAGUUCCAAGCA | cgtGCCAGTTCCAAG | caccGCCAGTTCCAA | aaacACAGTGTGCTT |
| Int39 | CACUGU (SEQ ID | CACACTGTGGGcag | GCACACTGT (SEQ ID | GGAACTGGC (SEQ |
| | NO: 287) | (SEQ ID NQ: 290) | NO: 293) | ID NO: 296) |
| sg- | GAUGGGGCGCUGG | gtgGATGGGGCGCTG | caccGATGGGGCGCT | aaacCACGATT CCAG |
| Int103 | AAUCGUG (SEQ ID | GAATCGTGAGGggc | GGAATCGTG (SEQ ID | CGCCCCATC (SEQ |
| | NO: 288) | (SEQ ID NO: 291) | NO: 294) | ID NO: 297) |
| sg- | GUGAGGAGCGUCU | gggGTGAGGAGCGTC | caccGTGAGGAGCGT | aaacGCT AGGCAGAC |
| Int153 | GCCUAGC (SEQ ID | TGCCTAGCAGGttc | CTGCCTAGC (SEQ ID | GCTCCTCAC (SEQ |
| | NO: 289) | (SEQ ID NO: 292) | NO: 295) | ID NO: 298) |

*The PAM sequence is indicated in bold. Nucleotides flanking the target site are indicated in lowercase.
^{§}Overhangs used for cloning are indicated in lowercase.

7.3.1.2. Evaluation of Indel Formation and Genomic Deletions

To evaluate deletion formation when targeting the RHO P347L locus in 293TetO-RHO-P347L cells using two guide RNAs, a PCR using primers spanning the predicted deletions (ICE-RHO_endo-F and ICE-RHO_plasmid-R, Table 11) was performed in order to verify the presence of lower molecular weight bands after agarose gel electrophoresis. To evaluate indel formation at intron targeted sites in 293TetO-RHO-P347L cells, primers ICE-RHO_endo2-F and ICE-RHO_plasmid-R were used (Table 11) to generate amplicon pools that were then Sanger sequenced for ICE analysis using the ICE-RHO_endo-R primer (ice.synthego.com).

7.3.2. Results: Design and Validation of Double-Cut-Based Strategy to Target the RHO P347L Mutation

An alternative strategy to knock-out the RHO P347L protein is represented by the induction of a targeted deletion of the P347L locus using two different guide RNAs (see scheme in FIG. 9A). One sgRNA overlaps the P347L mutation (Guide 1, presented in previous Examples) to confer allele-specificity to the targeting while a second sgRNA is positioned inside intron 4 of the RHO gene to promote the deletion. While the selection of the guide RNA on the P347L mutation is constrained by the position of the mutation itself, the sgRNA targeting intron 4 can be selected among all the SpCas9-compatible sgRNAs falling in that genomic region (see Table 2B). In selecting a target sequence from among the possible candidates, on-target efficacy, genome wide-specificity and position inside the intron can be considered in order to avoid adverse effects on the remaining RHO wild-type allele.

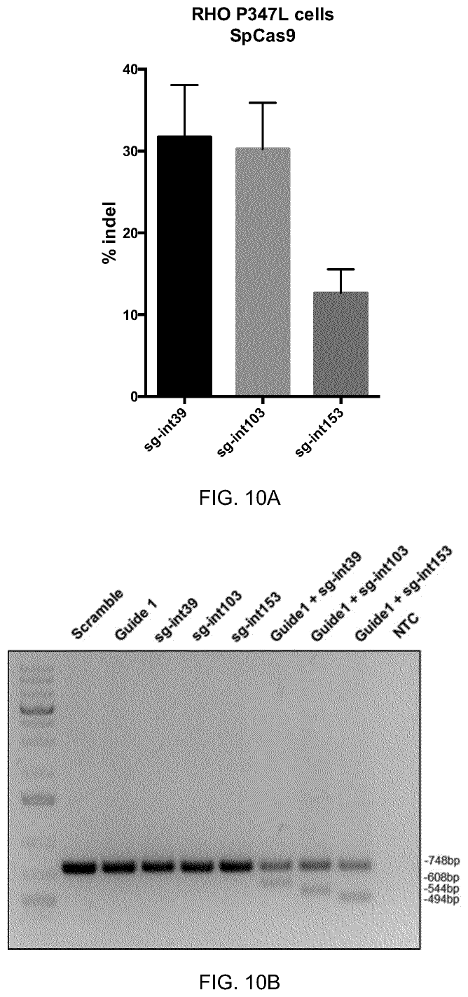

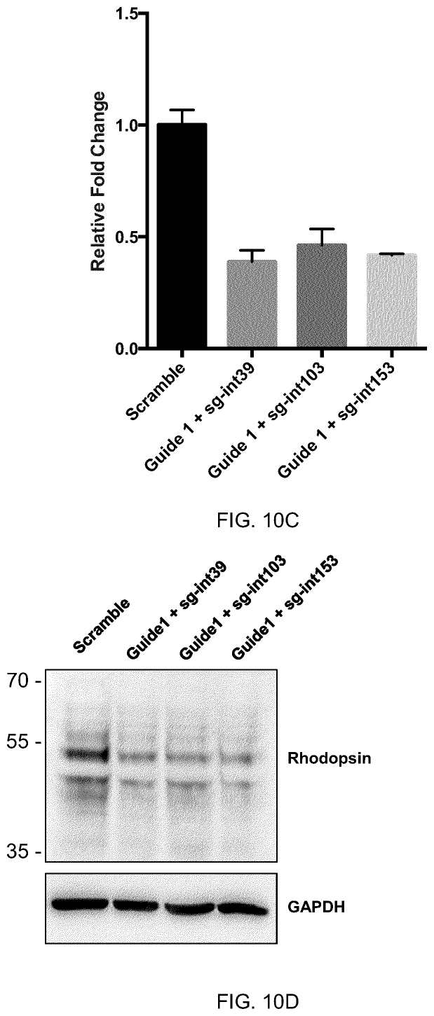

Initially, three different sgRNA targeting the 3′-end portion of RHO intron 4 were selected on the basis of their position and their overall predicted efficacy and specificity (using the CRISPOR website, crispor.org) (Concordet and Haeussler, 2018, Nucleic Acids Res. 46:W242-W245), see FIG. 9B and Table 13. The three guides (sg-Int39, sg-Int103, sg-Int153) were tested in combination with SpCas9 wt to evaluate indel formation at the target loci in 293TetO-RHO-P347L cells. As shown in FIG. 10A, wt SpCas9 demonstrated high editing levels in combination with all the three guides.

In order to further validate the targeting strategy, the induction of deletions was evaluated by PCR after transient transfection of 293TetO-RHO-P347L cells with each of the three intron-targeting guides in combination with Guide 1 (targeting the RHO P347L mutation) and wt SpCas9. The presence of the correct deleted product was detected with all the three combinations (FIG. 10B), in accordance with previous data showing good levels of indel formation at the respective targets when guides were tested singularly (FIG. 10A).

The effect of deletion formation on the levels of RHO P347L mRNA and protein was then evaluated in order to functionally validate the targeting strategy. As shown in FIG. 10C-D, treatment of 293TetO-RHO-P347L cells with Guide 1 in combination with each of the three intron-targeting guides produced significant downregulation of RHO P347L both at the level of mRNA (FIG. 100) and protein (FIG. 10D) thus demonstrating the efficacy of mutant RHO knockout through the deletion of part of RHO exon 5 using a dual-sgRNA strategy.

### 7.4. Example 4: RHO P347L Gene Editing with Nme2Cas9

7.4.1. Materials and Methods

The Material and Methods of the present Example are shared with those of Example 1. Additional Material and Methods specific for the present Example are reported here below.

Guide RNAs for Nme2Cas9 were cloned using oligonucleotides (Table 5) and methods disclosed in Example 1. The destination pUC19 plasmids containing the U6-driven expression cassette included either a standard scaffold (std) or a truncated scaffold (short) previously reported in literature (Sun et al., 2019, Mol. Cell 76:938-952.e5), see also Table 14.

TABLE 14
Nme2Cas9 RNA scaffold sequence (5′>3′)

| | |
|---|---|
| sgRNA | GUUGUAGCUCCCUUUCUCAUUUCGGAAACGAAAUGAGAACCG |
| std | UUGCUACAAUAAGGCCGUCUGAAAAGAUGUGCCGCAACGCUC |
| | UGCCCCUUAAAGCUUCUGCUUUAAGGGGCAUCGUUUA (SEQ |
| | ID NO: 199) |
| sgRNA | GUUGUAGCUCCCUUUCUCGAAAGAGAACCGUUGCUACAAUAA |
| short | GGCCGUCUGAAAAGAUGUGCCGCAACGCUCUGCCCCUUAAAG |
| | CUUCUGCUUUAAGGGGCAUCGUUUA (SEQ ID NO: 210) |

7.4.2. Results: Genome Editing at the RHO P347L Locus Using the Nme2Cas9 Small Ortholog

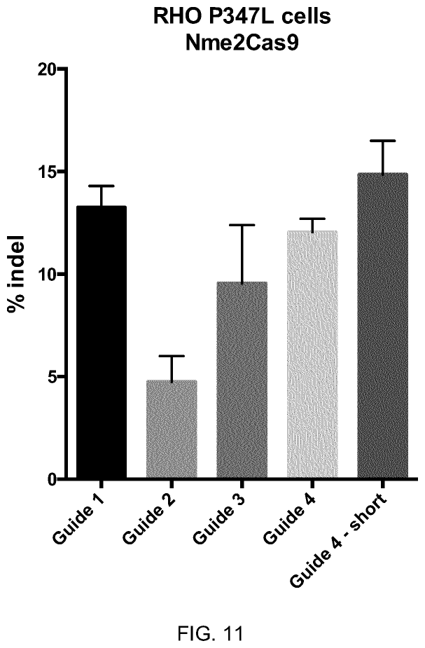

In order to evaluate the possibility to target the RHO P347L mutation using the small Cas9 ortholog Nme2Cas9 (Edraki et al., 2019, Mol. Cell 73:714-726.e4), a panel of guides spanning the mutated locus have been designed (see FIG. 1B) and tested by transient transfection in the 293T-RHO-P347L clone 4 cell line, stably expressing the mutated RHO P347L transgene. As shown in FIG. 11, indel formation was measured at the target locus with all the tested guides, with Guide 1 and Guide 4 being the most efficient. Additionally, to verify the possibility to further increase genome editing efficacy at the target locus, while reducing the overall size of the guide RNA molecule, Guide 4 was tested in combination with a truncated guide RNA scaffold (sgRNA short, Table 14). In these conditions, editing was preserved and slightly increased (FIG. 11).

### 8. SPECIFIC EMBODIMENTS

The present disclosure is exemplified by the specific embodiments below.

1. A guide RNA molecule (gRNA) for editing a human RHO gene having a P347 mutation.

2. The gRNA of embodiment 1, wherein the P347 mutation is a P347L mutation, a P347S mutation, a P347R mutation, a P347Q mutation, a P347T mutation, or a P347A mutation.

3. The gRNA of embodiment 2, wherein the P347 mutation is a P347L mutation.

4. The gRNA of embodiment 2, wherein the P347 mutation is a P347S mutation.

5. The gRNA of embodiment 2, wherein the P347 mutation is a P347R mutation.

6. The gRNA of embodiment 2, wherein the P347 mutation is a P347Q mutation.

7. The gRNA of embodiment 2, wherein the P347 mutation is a P347T mutation.

8. The gRNA of embodiment 2, wherein the P347 mutation is a P347A mutation.

9. The gRNA of any one of embodiments 1 to 8, wherein upon introduction of the gRNA and a DNA endonuclease capable of associating with the gRNA into a human cell containing a human RHO gene having a P347 mutation, the DNA endonuclease cleaves the RHO gene having a P347 mutation.

10. The gRNA of any one of embodiments 1 to 9, wherein upon introduction of the gRNA and a DNA endonuclease capable of associating with the gRNA into a human cell containing a human RHO gene having a P347 mutation and containing a human RHO gene not having a P347 mutation, indels in the human RHO gene not having a P347 mutation occur with a frequency of less than 20%.

11. The gRNA of any one of embodiments 1 to 9, wherein upon introduction of the gRNA and a DNA endonuclease capable of associating with the gRNA into a human cell containing a human RHO gene having a P347 mutation and containing a human RHO gene not having a P347 mutation, indels in the human RHO gene not having a P347 mutation occur with a frequency of less than 10%.

12. The gRNA of any one of embodiments 1 to 9, wherein upon introduction of the gRNA and a DNA endonuclease capable of associating with the gRNA into a human cell containing a human RHO gene having a P347 mutation and containing a human RHO gene not having a P347 mutation, indels in the human RHO gene not having a P347 mutation occur with a frequency of less than 1%.

13. The gRNA of any one of embodiments 1 to 9, wherein upon introduction of the gRNA and a DNA endonuclease capable of associating with the gRNA into a human cell containing a human RHO gene having a P347 mutation and containing a human RHO gene not having a P347 mutation, indels in the human RHO gene not having a P347 mutation occur with a frequency of less than 0.1%.

14. The gRNA of any one of embodiments 1 to 9, wherein upon introduction of the gRNA and a DNA endonuclease capable of associating with the gRNA into a population of human cells each containing a human RHO gene having a P347 mutation and containing a human RHO gene not having a P347 mutation, indels in the human RHO gene not having a P347 mutation occur with a frequency of less than 20%.

15. The gRNA of any one of embodiments 1 to 9, wherein upon introduction of the gRNA and a DNA endonuclease capable of associating with the gRNA into a population of human cells each containing a human RHO gene having a P347 mutation and containing a human RHO gene not having a P347 mutation, indels in the human RHO gene not having a P347 mutation occur with a frequency of less than 10%.

16. The gRNA of any one of embodiments 1 to 9, wherein upon introduction of the gRNA and a DNA endonuclease capable of associating with the gRNA into a population of human cells each containing a human RHO gene having a P347 mutation and containing a human RHO gene not having a P347 mutation, indels in the human RHO gene not having a P347 mutation occur with a frequency of less than 1%.

17. The gRNA of any one of embodiments 1 to 9, wherein upon introduction of the gRNA and a DNA endonuclease capable of associating with the gRNA into a population of human cells each containing a human RHO gene having a P347 mutation and containing a human RHO gene not having a P347 mutation, indels in the human RHO gene not having a P347 mutation occur with a frequency of less than 0.1%.

18. The gRNA of any one of embodiments 1 to 17, wherein upon introduction of the gRNA and a DNA endonuclease capable of associating with the gRNA into a human cell containing a human RHO gene having a P347 mutation and containing a human RHO gene not having a P347 mutation, the DNA endonuclease preferentially cleaves the RHO gene having a P347 mutation.

19. The gRNA of any one of embodiments 1 to 18, wherein upon introduction of the gRNA and a DNA endonuclease capable of associating with the gRNA into a population of human cells each containing a human RHO gene having a P347 mutation and containing a human RHO gene not having a P347 mutation, the DNA endonuclease preferentially cleaves the RHO gene having a P347 mutation.

20. The gRNA of embodiment 18 or embodiment 19, wherein the preferential cleavage of the RHO gene having a P347 mutation over the RHO gene not having a P347 mutation is by a factor of at least 1.3.

21. The gRNA of embodiment 18 or embodiment 19, wherein the preferential cleavage of the RHO gene having a P347 mutation over the RHO gene not having a P347 mutation is by a factor of at least 2.

22. The gRNA of embodiment 18 or embodiment 19, wherein the preferential cleavage of the RHO gene having a P347 mutation over the RHO gene not having a P347 mutation is by a factor of at least 2.5.

23. The gRNA of embodiment 18 or embodiment 19, wherein the preferential cleavage of the RHO gene having a P347 mutation over the RHO gene not having a P347 mutation is by a factor of at least 3.

24. The gRNA of embodiment 18 or embodiment 19, wherein the preferential cleavage of the RHO gene having a P347 mutation over the RHO gene not having a P347 mutation is by a factor of at least 4.

25. The gRNA of embodiment 18 or embodiment 19, wherein the preferential cleavage of the RHO gene having a P347 mutation over the RHO gene not having a P347 mutation is by a factor of at least 5.

26. The gRNA of embodiment 18 or embodiment 19, wherein the preferential cleavage of the RHO gene having a P347 mutation over the RHO gene not having a P347 mutation is by a factor of at least 10.

27. The gRNA of embodiment 18 or embodiment 19, wherein the preferential cleavage of the RHO gene having a P347 mutation over the RHO gene not having a P347 mutation is by a factor of at least 100.

28. The gRNA of any one of embodiments 20 to 25, wherein the preferential cleavage of the RHO gene having a P347 mutation over the RHO gene not having a P347 mutation is by a factor of up to 5.

29. The gRNA of any one of embodiments 20 to 26, wherein the preferential cleavage of the RHO gene having a P347 mutation over the RHO gene not having a P347 mutation is by a factor of up to 10.

30. The gRNA of any one of embodiments 20 to 26, wherein the preferential cleavage of the RHO gene having a P347 mutation over the RHO gene not having a P347 mutation is by a factor of up to 11.

31. The gRNA of any one of embodiments 9 to 30, wherein the human cell or population of human cells is a HEK293T/17 cell or a population of HEK293T/17 cells.

32. The gRNA of any one of embodiments 1 to 31, which comprises a spacer that is 15 to 30 nucleotides in length.

33. The gRNA of embodiment 32, wherein the spacer is 15 to 25 nucleotides in length.

34. The gRNA of embodiment 32, wherein the spacer is 16 to 24 nucleotides in length.

35. The gRNA of embodiment 32, wherein the spacer is 17 to 23 nucleotides in length.

36. The gRNA of embodiment 32, wherein the spacer is 18 to 22 nucleotides in length.

37. The gRNA of embodiment 32, wherein the spacer is 19 to 21 nucleotides in length.

38. The gRNA of embodiment 32, wherein the spacer is 18 to 30 nucleotides in length.

39. The gRNA of embodiment 32, wherein the spacer is 20 to 28 nucleotides in length.

40. The gRNA of embodiment 32, wherein the spacer is 22 to 26 nucleotides in length.

41. The gRNA of embodiment 32, wherein the spacer is 23 to 25 nucleotides in length.

42. The gRNA of embodiment 32, wherein the spacer is 20 nucleotides in length.

43. The gRNA of embodiment 32, wherein the spacer is 21 nucleotides in length.

44. The gRNA of embodiment 32, wherein the spacer is 22 nucleotides in length.

45. The gRNA of embodiment 32, wherein the spacer is 23 nucleotides in length.

46. The gRNA of embodiment 32, wherein the spacer is 24 nucleotides in length.

47. The gRNA of embodiment 32, wherein the spacer is 25 nucleotides in length.

48. The gRNA of any one of embodiments 32 to 47, wherein the nucleotide sequence of the spacer comprises 15 or more consecutive nucleotides of a reference sequence or comprises a nucleotide sequence that is at least 85% identical to a reference sequence, wherein the reference sequence is:

(SEQ ID NO: 5)
a) GGUCUUAGGCCAGGGCCACC;
(SEQ ID NO: 6)
b) GCCCUGGCCUAAGACCUGCCU;
(SEQ ID NO: 7)
c) CCUAGGCAGGUCUUAGGCCA;
(SEQ ID NO: 8)
d) gCCUAGGCAGGUCUUAGGCCA;
(SEQ ID NO: 9)
e) GACGAGCCAGGUGGCCCUGGCCU;
(SEQ ID NO: 10)
f) GUCCUAGGCAGGUCUUAGGCCAGG;
(SEQ ID NO: 11)
g) UUAGGCCAGGGCCACCUGGCUC;
or
(SEQ ID NO: 12)
h) GCCAGGUGGCCCUGGCCUAAGA.

49. A guide RNA molecule (gRNA) comprising a spacer whose nucleic acid sequence comprises 15 or more consecutive nucleotides from a reference sequence or comprises a nucleotide sequence that is at least 85% identical to a reference sequence, wherein the reference sequence is:

(SEQ ID NO: 5)
a) GGUCUUAGGCCAGGGCCACC;
(SEQ ID NO: 6)
b) GCCCUGGCCUAAGACCUGCCU;
(SEQ ID NO: 7)
c) CCUAGGCAGGUCUUAGGCCA;
(SEQ ID NO: 8)
d) gCCUAGGCAGGUCUUAGGCCA;
(SEQ ID N0: 9)
e) GACGAGCCAGGUGGCCCUGGCCU;
(SEQ ID NO: 10)
f) GUCCUAGGCAGGUCUUAGGCCAGG;
(SEQ ID NO: 11)
g) UUAGGCCAGGGCCACCUGGCUC;
or
(SEQ ID NO: 12)
h) GCCAGGUGGCCCUGGCCUAAGA.

50. The gRNA molecule of embodiment 48 or embodiment 49, wherein the nucleotide sequence of the spacer comprises 15 or more consecutive nucleotides from the reference sequence.

51. The gRNA molecule of embodiment 48 or embodiment 49, wherein the nucleotide sequence of the spacer comprises 16 or more consecutive nucleotides from the reference sequence.

52. The gRNA molecule of embodiment 48 or embodiment 49, wherein the nucleotide sequence of the spacer comprises 17 or more consecutive nucleotides from the reference sequence.

53. The gRNA molecule of embodiment 48 or embodiment 49, wherein the nucleotide sequence of the spacer comprises 18 or more consecutive nucleotides from the reference sequence.

54. The gRNA molecule of embodiment 48 or embodiment 49, wherein the nucleotide sequence of the spacer comprises 19 or more consecutive nucleotides from the reference sequence

55. The gRNA molecule of embodiment 48 or embodiment 49, wherein the nucleotide sequence of the spacer comprises 20 or more consecutive nucleotides from the reference sequence.

56. The gRNA molecule of embodiment 48 or embodiment 49, wherein the nucleotide sequence of the spacer comprises 21 or more consecutive nucleotides from the reference sequence.

57. The gRNA molecule of embodiment 48 or embodiment 49, wherein the nucleotide sequence of the spacer comprises 22 or more consecutive nucleotides from the reference sequence.

58. The gRNA molecule of embodiment 48 or embodiment 49, wherein the nucleotide sequence of the spacer comprises 23 or more consecutive nucleotides from the reference sequence.

59. The gRNA molecule of embodiment 48 or embodiment 49, wherein the nucleotide sequence of the spacer comprises a sequence that is at least 85% identical to the reference sequence.

60. The gRNA molecule of embodiment 48 or embodiment 49, wherein the nucleotide sequence of the spacer comprises a sequence that is at least 90% identical to the reference sequence.

61. The gRNA molecule of embodiment 48 or embodiment 49, wherein the nucleotide sequence of the spacer comprises a sequence that is at least 95% identical to the reference sequence.

62. The gRNA molecule of embodiment 48 or embodiment 49, wherein the nucleotide sequence of the spacer comprises a sequence that is identical to the reference sequence.

63. The gRNA molecule of embodiment 48 or embodiment 49, wherein the nucleotide sequence of the spacer comprises a sequence that has one or two mismatches to the reference sequence and corresponding to a P347 mutation (e.g., a P347S mutation, a P347R mutation, a P347Q mutation, a P347T mutation, or a P347A mutation), but is otherwise identical to the reference sequence.

64. The gRNA molecule of any one of embodiments 48 to 63, wherein the reference sequence is GGUCUUAGGCCAGGGCCACC (SEQ ID NO:5).

65. The gRNA molecule of any one of embodiments 48 to 63, wherein the reference sequence is GCCCUGGCCUAAGACCUGCCU (SEQ ID NO:6).

66. The gRNA molecule of any one of embodiments 48 to 63, wherein the reference sequence is CCUAGGCAGGUCUUAGGCCA (SEQ ID NO:7).

67. The gRNA molecule of any one of embodiments 48 to 63, wherein the reference sequence is gCCUAGGCAGGUCUUAGGCCA (SEQ ID NO:8).

68. The gRNA molecule of any one of embodiments 48 to 63, wherein the reference sequence is GACGAGCCAGGUGGCCCUGGCCU (SEQ ID NO:9).

69. The gRNA molecule of any one of embodiments 48 to 63, wherein the reference sequence is GUCCUAGGCAGGUCUUAGGCCAGG (SEQ ID NO:10).

70. The gRNA molecule of any one of embodiments 48 to 63, wherein the reference sequence is UUAGGCCAGGGCCACCUGGCUC (SEQ ID NO:11).

71. The gRNA molecule of any one of embodiments 48 to 63, wherein the reference sequence is GCCAGGUGGCCCUGGCCUAAGA (SEQ ID NO:12).

72. The gRNA molecule of any one of embodiments 1 to 71, which is a Cas9 gRNA.

73. The gRNA molecule of embodiment 72, which is a Streptococcus pyogenes Cas9 (SpCas9) gRNA.

74. The gRNA molecule of embodiment 72, which is a Neisseria meningitidis Cas9 gRNA.

75. The gRNA molecule of embodiment 74, which is a Nme2Cas9 gRNA.

76. The gRNA molecule of any one of embodiments 1 to 75, which is a single guide RNA (sgRNA).

77. The gRNA molecule of embodiment 76, which comprises a 3′ sgRNA segment.

78. The gRNA molecule of embodiment 77, wherein the 3′ sgRNA segment has a nucleotide sequence comprising a sequence set forth in Table 3 or Table 4.

79. The gRNA of embodiment 78, wherein the 3′ sgRNA segment comprises the nucleotide sequence of 3′ sgRNA sequence 1 as set forth in Table 3.

80. The gRNA of embodiment 78, wherein the 3′ sgRNA segment comprises the nucleotide sequence of 3′ sgRNA sequence 2 as set forth in Table 3.

81. The gRNA of embodiment 78, wherein the 3′ sgRNA segment comprises the nucleotide sequence of 3′ sgRNA sequence 3 as set forth in Table 3.

82. The gRNA of embodiment 78, wherein the 3′ sgRNA segment comprises the nucleotide sequence of 3′ sgRNA sequence 4 as set forth in Table 3.

83. The gRNA of embodiment 78, wherein the 3′ sgRNA segment comprises the nucleotide sequence of 3′ sgRNA sequence 5 as set forth in Table 3.

84. The gRNA of embodiment 78, wherein the 3′ sgRNA segment comprises the nucleotide sequence of 3′ sgRNA sequence 6 as set forth in Table 3.

85. The gRNA of embodiment 78, wherein the 3′ sgRNA segment comprises the nucleotide sequence of 3′ sgRNA sequence 7 as set forth in Table 3.

86. The gRNA of embodiment 78, wherein the 3′ sgRNA segment comprises the nucleotide sequence of 3′ sgRNA sequence 8 as set forth in Table 3.

87. The gRNA of embodiment 78, wherein the 3′ sgRNA segment comprises the nucleotide sequence of 3′ sgRNA sequence 9 as set forth in Table 3.

88. The gRNA of embodiment 78, wherein the 3′ sgRNA segment comprises the nucleotide sequence of 3′ sgRNA sequence 10 as set forth in Table 3.

89. The gRNA of embodiment 78, wherein the 3′ sgRNA segment comprises the nucleotide sequence of 3′ sgRNA sequence 11 as set forth in Table 3.

90. The gRNA of embodiment 78, wherein the 3′ sgRNA segment comprises the nucleotide sequence of 3′ sgRNA sequence 12 as set forth in Table 3.

91. The gRNA of embodiment 78, wherein the 3′ sgRNA segment comprises the nucleotide sequence of 3′ sgRNA sequence 13 as set forth in Table 3.

92. The gRNA of embodiment 78, wherein the 3′ sgRNA segment comprises the nucleotide sequence of 3′ sgRNA sequence 14 as set forth in Table 3.

93. The gRNA of embodiment 78, wherein the 3′ sgRNA segment comprises the nucleotide sequence of 3′ sgRNA sequence 15 as set forth in Table 3.

94. The gRNA of embodiment 78, wherein the 3′ sgRNA segment comprises the nucleotide sequence of 3′ sgRNA sequence 16 as set forth in Table 3.

95. The gRNA of embodiment 78, wherein the 3′ sgRNA segment comprises the nucleotide sequence of 3′ sgRNA sequence 17 as set forth in Table 3.

96. The gRNA of embodiment 78, wherein the 3′ sgRNA segment comprises the nucleotide sequence of 3′ sgRNA sequence 18 as set forth in Table 3.

97. The gRNA of embodiment 78, wherein the 3′ sgRNA segment comprises the nucleotide sequence of 3′ sgRNA sequence 19 as set forth in Table 3.

98. The gRNA of embodiment 78, wherein the 3′ sgRNA segment comprises the nucleotide sequence of 3′ sgRNA sequence 20 as set forth in Table 3.

99. The gRNA of embodiment 78, wherein the 3′ sgRNA segment comprises the nucleotide sequence of 3′ sgRNA sequence 21 as set forth in Table 3.

100. The gRNA of embodiment 78, wherein the 3′ sgRNA segment comprises the nucleotide sequence of 3′ sgRNA sequence 22 as set forth in Table 3.

101. The gRNA of embodiment 78, wherein the 3′ sgRNA segment comprises the nucleotide sequence of 3′ sgRNA sequence 23 as set forth in Table 3.

102. The gRNA of embodiment 78, wherein the 3′ sgRNA segment comprises the nucleotide sequence of 3′ sgRNA sequence 24 as set forth in Table 3.

103. The gRNA of embodiment 78, wherein the 3′ sgRNA segment comprises the nucleotide sequence of 3′ sgRNA sequence 25 as set forth in Table 3.

104. The gRNA of embodiment 78, wherein the 3′ sgRNA segment comprises the nucleotide sequence of 3′ sgRNA sequence 26 as set forth in Table 3.

105. The gRNA of embodiment 78, wherein the 3′ sgRNA segment comprises the nucleotide sequence of 3′ sgRNA sequence 27 as set forth in Table 3.

106. The gRNA of embodiment 78, wherein the 3′ sgRNA segment comprises the nucleotide sequence of 3′ sgRNA sequence 28 as set forth in Table 3.

107. The gRNA of embodiment 78, wherein the 3′ sgRNA segment comprises the nucleotide sequence of 3′ sgRNA sequence 29 as set forth in Table 3.

108. The gRNA of embodiment 78, wherein the 3′ sgRNA segment comprises the nucleotide sequence of 3′ sgRNA sequence 30 as set forth in Table 3.

109. The gRNA of embodiment 78, wherein the 3′ sgRNA segment comprises the nucleotide sequence of 3′ sgRNA sequence 31 as set forth in Table 3.

110. The gRNA of embodiment 78, wherein the 3′ sgRNA segment comprises the nucleotide sequence of 3′ sgRNA sequence 1 as set forth in Table 4.

111. The gRNA of embodiment 78, wherein the 3′ sgRNA segment comprises the nucleotide sequence of 3′ sgRNA sequence 2 as set forth in Table 4.

112. The gRNA of embodiment 78, wherein the 3′ sgRNA segment comprises the nucleotide sequence of 3′ sgRNA sequence 3 as set forth in Table 4.

113. The gRNA of embodiment 78, wherein the 3′ sgRNA segment comprises the nucleotide sequence of 3′ sgRNA sequence 4 as set forth in Table 4.

114. The gRNA of embodiment 78, wherein the 3′ sgRNA segment comprises the nucleotide sequence of 3′ sgRNA sequence 5 as set forth in Table 4.

115. The gRNA of embodiment 78, wherein the 3′ sgRNA segment comprises the nucleotide sequence of 3′ sgRNA sequence 6 as set forth in Table 4.

116. The gRNA of embodiment 78, wherein the 3′ sgRNA segment comprises the nucleotide sequence of 3′ sgRNA sequence 7 as set forth in Table 4.

117. The gRNA of embodiment 78, wherein the 3′ sgRNA segment comprises the nucleotide sequence of 3′ sgRNA sequence 8 as set forth in Table 4.

118. The gRNA of embodiment 78, wherein the 3′ sgRNA segment comprises the nucleotide sequence of 3′ sgRNA sequence 9 as set forth in Table 4.

119. The gRNA of embodiment 78, wherein the 3′ sgRNA segment comprises the nucleotide sequence of 3′ sgRNA sequence 10 as set forth in Table 4.

120. The gRNA of embodiment 78, wherein the 3′ sgRNA segment comprises the nucleotide sequence of 3′ sgRNA sequence 11 as set forth in Table 4.

121. The gRNA of embodiment 78, wherein the 3′ sgRNA segment comprises the nucleotide sequence of 3′ sgRNA sequence 12 as set forth in Table 4.

122. The gRNA of embodiment 78, wherein the 3′ sgRNA segment comprises the nucleotide sequence of 3′ sgRNA sequence 13 as set forth in Table 4.

123. The gRNA of embodiment 78, wherein the 3′ sgRNA segment comprises the nucleotide sequence of 3′ sgRNA sequence 14 as set forth in Table 4.

124. The gRNA of embodiment 78, wherein the 3′ sgRNA segment comprises the nucleotide sequence of 3′ sgRNA sequence 15 as set forth in Table 4.

125. The gRNA of embodiment 78, wherein the 3′ sgRNA segment comprises the nucleotide sequence of 3′ sgRNA sequence 16 as set forth in Table 4.

126. The gRNA of embodiment 78, wherein the 3′ sgRNA segment comprises the nucleotide sequence of 3′ sgRNA sequence 17 as set forth in Table 4.

127. The gRNA of embodiment 78, wherein the 3′ sgRNA segment comprises the nucleotide sequence of 3′ sgRNA sequence 18 as set forth in Table 4.

128. The gRNA of embodiment 78, wherein the 3′ sgRNA segment comprises the nucleotide sequence of 3′ sgRNA sequence 19 as set forth in Table 4.

129. The gRNA of embodiment 78, wherein the 3′ sgRNA segment comprises the nucleotide sequence of 3′ sgRNA sequence 20 as set forth in Table 4.

130. The gRNA of embodiment 78, wherein the 3′ sgRNA segment comprises the nucleotide sequence of 3′ sgRNA sequence 21 as set forth in Table 4.

131. The gRNA of embodiment 78, wherein the 3′ sgRNA segment comprises the nucleotide sequence of 3′ sgRNA sequence 22 as set forth in Table 4.

132. The gRNA of any one of embodiments 77 to 131, wherein the 3′ sgRNA segment comprises one or more uracils at its 3′ end.

133. The gRNA of embodiment 132, wherein the 3′ sgRNA segment comprises one to eight uracils at its 3′ end.

134. The gRNA of embodiment 133, wherein the 3′ sgRNA segment comprises one uracil at its 3′ end.

135. The gRNA of embodiment 133, wherein the 3′ sgRNA segment comprises two uracils at its 3′ end.

136. The gRNA of embodiment 133, wherein the 3′ sgRNA segment comprises three uracils at its 3′ end.

137. The gRNA of embodiment 133, wherein the 3′ sgRNA segment comprises four uracils at its 3′ end.

138. The gRNA of embodiment 133, wherein the 3′ sgRNA segment comprises five uracils at its 3′ end.

139. The gRNA of embodiment 133, wherein the 3′ sgRNA segment comprises six uracils at its 3′ end.

140. The gRNA of embodiment 133, wherein the 3′ sgRNA segment comprises seven uracils at its 3′ end.

141. The gRNA of embodiment 133, wherein the 3′ sgRNA segment comprises eight uracils at its 3′ end.

142. The gRNA of any one of embodiments 1 to 141, wherein the gRNA is an unmodified gRNA.

143. The gRNA of any one of embodiments 1 to 141, which comprises one or more modifications.

144. The gRNA of embodiment 143, wherein the one or more modifications comprises one or more 2′-O-methyl phosphorothioate nucleotides.

145. A nucleic acid encoding the gRNA of any one of embodiments 1 to 142.

146. The nucleic acid of embodiment 145, which further comprises a Pol III promoter sequence operably linked to the nucleotide sequence encoding the gRNA.

147. The nucleic acid of embodiment 146, wherein the promoter is a U6 promoter.

148. The nucleic acid of embodiment 146, wherein the promoter is a H1 promoter.

149. The nucleic acid of any one of embodiments 145 to 148, which further encodes a second gRNA.

150. The nucleic acid of embodiment 149, wherein the second gRNA is a sgRNA.

151. The nucleic acid of embodiment 149 or embodiment 150, wherein the second gRNA comprises a spacer sequence that is partially or fully complementary to a target sequence in intron 4 of a human RHO gene.

152. The nucleic acid of embodiment 151, wherein the second gRNA comprises a spacer sequence corresponding to a sequence set forth in Table 2B.

153. The nucleic acid of any one of embodiments 151 to 152, wherein the spacer sequence of the second gRNA comprises the spacer sequence of sg-Int39 as set forth in Table 13.

154. The nucleic acid of any one of embodiments 151 to 152, wherein the spacer sequence of the second gRNA comprises the spacer sequence of sg-Int103 as set forth in Table 13.

155. The nucleic acid of any one of embodiments 151 to 152, wherein the spacer sequence of the second gRNA comprises the spacer sequence of sg-Int153 as set forth in Table 13.

156. The nucleic acid of any one of embodiments 145 to 155, which further encodes a Cas9 protein.

157. The nucleic acid of embodiment 156, which further comprises a tissue-specific promoter sequence operably linked to the nucleotide sequence encoding the Cas9 protein.

158. The nucleic acid of embodiment 157, wherein the tissue-specific promoter operably linked to the nucleotide sequence encoding the Cas9 protein is a RHO promoter.

159. The nucleic acid of embodiment 157, wherein the tissue-specific promoter operably linked to the nucleotide sequence encoding the Cas9 protein is a hGRK1 promoter.

160. The nucleic acid of embodiment 156, which further comprises a constitutive promoter sequence operably linked to the nucleotide sequence encoding the Cas9 protein.

161. The nucleic acid of embodiment 160, wherein the constitutive promoter is an EF1 alpha promoter, e.g., EF1 alpha short (EFS) promoter.

162. The nucleic acid of any one of embodiments 156 to 161, wherein the Cas9 protein is a SpCas9 protein or a SpCas9 protein variant.

163. The nucleic acid of any one of embodiments 156 to 161, wherein the Cas9 protein is a Nme2Cas9 protein or a Nme2Cas9 protein variant.

164. The nucleic acid of any one of embodiments 156 to 163, wherein the Cas9 protein is a wild-type Cas9 protein.

165. The nucleic acid of any one of embodiments 156 to 163, wherein the Cas9 protein is a Cas9 protein variant having one or more amino acid modifications relative to the corresponding wild-type Cas9 protein.

166. The nucleic acid of embodiment 165, wherein the wherein the positions of one or more mutations are identified by reference to the amino acid numbering in an unmodified mature Streptococcus pyogenes Cas9 as set forth in SEQ ID NO:1.

167. The nucleic acid of embodiment 166, wherein the Cas9 variant comprises a K526 mutation.

168. The nucleic acid of embodiment 167, wherein the K526 mutation is a K526E mutation.

169. The nucleic acid of embodiment 167, wherein the K526 mutation is a K526N mutation.

170. The nucleic acid of any one of embodiments 166 to 169, wherein the Cas9 variant comprises a Y515 mutation.

171. The nucleic acid of embodiment 170, wherein the Y515 mutation is a Y515N mutation.

172. The nucleic acid of any one of embodiments 166 to 171, wherein the Cas9 variant comprises a R661 mutation.

173. The nucleic acid of embodiment 172, wherein the R661 mutation is a R661Q mutation.

174. The nucleic acid of embodiment 172, wherein the R661 mutation is a R661S mutation.

175. The nucleic acid of any one of embodiments 166 to 174, wherein the Cas9 variant comprises a M495 mutation.

176. The nucleic acid of embodiment 175, wherein the M495 mutation is M495V mutation.

177. The nucleic acid of embodiment 166, wherein the Cas9 variant comprises K526E and R661S mutations.

178. The nucleic acid of embodiment 166, wherein the Cas9 variant comprises K526E and R661Q mutations.

179. The nucleic acid of embodiment 166, wherein the Cas9 variant comprises Y515N, K526E, and R661S mutations.

180. The nucleic acid of embodiment 166, wherein the Cas9 variant comprises Y515N, K526E, and R661Q mutations.

181. The nucleic acid of embodiment 166, wherein the Cas9 variant comprises M495V, Y515N, K526E, and R661Q mutations.

182. The nucleic acid of embodiment 166, wherein the Cas9 variant comprises M495V, Y515N, K526E, and R661S mutations.

183. The nucleic acid of embodiment 166, wherein the Cas9 variant comprises N692A, M694A, Q695A, H698A mutations.

184. The nucleic acid of embodiment 166, wherein the Cas9 variant comprises K848A, K1003A, and R1060A mutations.

185. The nucleic acid of embodiment 166, wherein the Cas9 variant comprises F539S, M763I, and K890N mutations.

186. The nucleic acid of embodiment 166, wherein the Cas9 variant comprises N497A, R661A, Q695A, and Q926A mutations.

187. The nucleic acid of embodiment 166, wherein the Cas9 variant comprises a R691A mutation.

188. The nucleic acid of any one of embodiments 145 to 187, which is a plasmid.

189. The nucleic acid of any one of embodiments 145 to 187, which is a viral genome.

190. The nucleic acid of embodiment 189, wherein the viral genome is an adeno-associated virus (AAV) genome.

191. The nucleic acid of embodiment 190, wherein the AAV genome is a AAV2, AAV5, or AAV8 genome.

192. The nucleic acid of embodiment 190, wherein the AAV genome is a AAV7m8 genome.

193. The nucleic acid of embodiment 191, wherein the AAV genome is a AAV2 genome.

194. The nucleic acid of embodiment 191, wherein the AAV genome is a AAV5 genome.

195. The nucleic acid of embodiment 191, wherein the AAV genome is a AAV8 genome.

196. A plurality of nucleic acids comprising (a) the gRNA of any one of embodiments 1 to 144 or the nucleic acid of any one of embodiments 145 to 195, and (b) a nucleic acid encoding a Cas9 protein.

197. The plurality of nucleic acids of embodiment 196, wherein the Cas9 protein has the features of a Cas9 protein described in any one of embodiments 162 to 187.

198. The plurality of nucleic acids of embodiment 196 or embodiment 197, further comprising a nucleic acid encoding a second gRNA.

199. The plurality of nucleic acids of embodiment 198, wherein the second gRNA has the features of a second gRNA described in any one of embodiments 150 to 155.

200. A particle comprising the gRNA of any one of embodiments 1 to 144, the nucleic acid of any one of embodiments 145 to 195, or the plurality of nucleic acids of any one of embodiments 196 to 199.

201. The particle of embodiment 200, which further comprises a Cas9 protein or a nucleic acid encoding a Cas9 protein.

202. The particle of embodiment 201, wherein the Cas9 protein has the features of a Cas9 protein described in any one of embodiments 162 to 187.

203. The particle of any one of embodiments 200 to 202, which is a lipid nanoparticle, a vesicle, or a gold nanoparticle.

204. The particle of any one of embodiments 200 to 203, which comprises a single species of gRNA or comprises a nucleic acid encoding a single species of gRNA.

205. The particle of any one of embodiments 200 to 203, which comprises more than one species of gRNA or comprises a nucleic acid encoding more than one species of gRNA.

206. A particle comprising the nucleic acid of any one of embodiments 145 to 195, wherein the particle is a viral particle.

207. The particle of embodiment 206, which is an adeno-associated virus (AAV) particle.

208. The particle of embodiment 207, which is a AAV2 particle.

209. The particle of embodiment 207, which is a AAV5 particle.

210. The particle of embodiment 207, which is a AAV7m8 particle.

211. The particle of embodiment 207, which is a AAV8 particle.

212. The particle of any one of embodiments 206 to 211, which comprises a nucleic acid encoding a Cas9 protein.

213. The particle of embodiment 212, wherein the Cas9 protein has the features of a Cas9 protein described in any one of embodiments 162 to 187.

214. A plurality of particles comprising the particle of any one of embodiments 206 to 211 and a particle comprising a nucleic acid encoding a Cas9 protein.

215. The plurality of particles of embodiment 214, wherein the Cas9 protein has the features of a Cas9 protein described in any one of embodiments 162 to 187.

216. The plurality of particles of embodiment 214 or embodiment 215, wherein the particle comprising a nucleic acid encoding the Cas9 protein is a viral particle.

217. The plurality of particles of embodiment 216, wherein the particle comprising a nucleic acid encoding the Cas9 protein is an adeno-associated virus (AAV) particle.

218. The plurality of particles of embodiment 217, wherein the particle comprising a nucleic acid encoding the Cas9 protein is a AAV2 particle.

219. The plurality of particles of embodiment 217, wherein the particle comprising a nucleic acid encoding the Cas9 protein is a AAV5 particle.

220. The plurality of particles of embodiment 217, wherein the particle comprising a nucleic acid encoding the Cas9 protein is a AAV7m8 particle.

221. The plurality of particles of embodiment 217, wherein the particle comprising a nucleic acid encoding the Cas9 protein is a AAV8 particle.

222. A system comprising a Cas9 protein and a gRNA of any one of embodiments 1 to 144.

223. The system of embodiment 222, wherein the Cas9 protein has the features of a Cas9 protein described in any one of embodiments 162 to 187.

224. The system of embodiment 222 or embodiment 223, further comprising a second gRNA.

225. The system of embodiment 224, wherein the second gRNA has the features of a second gRNA described in any one of embodiments 150 to 155.

226. The system of any one of embodiments 222 to 225, further comprising genomic DNA comprising a RHO gene having a P347 mutation.

227. A pharmaceutical composition comprising (i) the gRNA of any one of embodiments 1 to 144, the nucleic acid of any one of embodiments 145 to 195, the plurality of nucleic acids of any one of embodiments 196 to 199, the particle of any one of embodiments 200 to 213, the plurality of particles of any one of embodiments 214 to 221, or the system of any one of embodiments 222 to 226 and (ii) a pharmaceutically acceptable excipient.

228. A cell comprising the gRNA of any one of embodiments 1 to 144.

229. A cell comprising the nucleic acid of any one of embodiments 145 to 195.

230. A cell comprising the plurality of nucleic acids of any one of embodiments 196 to 199.

231. A cell comprising the particle of any one of embodiments 200 to 213.

232. A cell comprising the plurality of particles of any one of embodiments 214 to 221.

233. A cell comprising the system of any one of embodiments 222 to 226.

234. The cell of any one of embodiments 228 to 233, which is a human cell.

235. The cell of embodiment 234, which is a human retinal cell.

236. The cell of embodiment 234, which is a human retinal epithelial cell.

237. The cell of embodiment 234, which is a human photoreceptor cell.

238. The cell of embodiment 234, which is a human retinal progenitor cell.

239. The cell of embodiment 234, which is a stem cell.

240. The cell of embodiment 234, which is an iPS cell.

241. The cell of embodiment 234, which is a HEK293T cell.

242. The cell of embodiment 241, which is a HEK293T/17 cell.

243. The cell of any one of embodiments 228 to 242, which is an ex vivo cell.

244. A population of cells according to any one of embodiments 228 to 243.

245. A method of altering a human cell comprising a RHO gene having a P347 mutation, comprising contacting the cell with the gRNA of any one of embodiments 1 to 144, the nucleic acid of any one of embodiments 145 to 195, the plurality of nucleic acids of any one of embodiments 196 to 199, the particle of any one of embodiments 200 to 213, the plurality of particles of any one of embodiments 214 to 221, the system of any one of embodiments 222 to 226 or the pharmaceutical composition of embodiment 227.

246. The method of embodiment 245, wherein the P347 mutation is a P347L mutation, a P347S mutation, a P347R mutation, a P347Q mutation, a P347T mutation, or a P347A mutation.

247. The method of embodiment 246, wherein the P347 mutation is a P347L mutation.

248. The method of embodiment 246, wherein the P347 mutation is a P347S mutation.

249. The method of embodiment 246, wherein the P347 mutation is a P347R mutation.

250. The method of embodiment 246, wherein the P347 mutation is a P347Q mutation.

251. The method of embodiment 246, wherein the P347 mutation is a P347T mutation.

252. The method of embodiment 246, wherein the P347 mutation is a P347A mutation.

253. The method of any one of embodiments 245 to 252, which comprises contacting the cell with the particle of any one of embodiments 200 to 213 or the plurality of particles of any one of embodiments 214 to 221.

254. The method of any one of embodiments 245 to 252, which comprises contacting the cell with the system of any one of embodiments 222 to 226.

255. The method of embodiment 254, wherein the contacting comprises delivering the system to the cell via one or more particles and/or one or more vectors.

256. The method of embodiment 255, wherein the contacting comprises delivering the system to the cell via one or more particles.

257. The method of embodiment 255, wherein the one or more particles comprise a lipid nanoparticle, a vesicle, or a gold nanoparticle.

258. The method of any one of embodiments 255 to 257, wherein the contacting comprises delivering the system to the cell via one or more vectors.

259. The method of embodiment 258, wherein the one or more vectors comprise one or more viral vectors.

260. The method of embodiment 259, wherein the one or more viral vectors comprise a lentivirus, an adenovirus, or an adeno-associated virus.

261. The method of embodiment 260, wherein the one or more viral vectors comprise a lentivirus.

262. The method of embodiment 260, wherein the one or more viral vectors comprise an adenovirus.

263. The method of embodiment 260, wherein the one or more viral vectors comprise an adeno-associated virus (AAV).

264. The method of embodiment 263, wherein the one or more viral vectors comprise one or more AAV2, AAV5 or AAV8 vectors.

265. The method of embodiment 263, wherein the one or more viral vectors comprise one or more AAV2, AAV5 AAV7m8, or AAV8 vectors

266. The method of embodiment 264 or embodiment 265, wherein the one or more viral vectors comprise one or more AAV2 vectors.

267. The method of embodiment 264 or embodiment 265, wherein the one or more viral vectors comprise one or more AAV5 vectors.

268. The method of embodiment 265, wherein the one or more viral vectors comprise one or more AAV7m8 vectors.

269. The method of embodiment 264 or embodiment 265, wherein the one or more viral vectors comprise one or more AAV8 vectors.

270. The method of any one of embodiments 258 to 269, wherein the one or more viral vectors comprise nucleic acid(s) encoding the gRNA(s) and the Cas9 protein each operably linked to a promoter.

271. The method of embodiment 270, wherein the nucleic acid encoding the gRNA(s) is/are operably linker to a Pol III promoter.

272. The method of embodiment 271, wherein the Pol III promoter is a U6 promoter.

273. The method of embodiment 271, wherein the Pol III promoter is a H1 promoter.

274. The method of any one of embodiments 270 to 273, wherein the nucleic acid encoding the Cas9 protein is operably linked to a tissue specific promoter.

275. The method of embodiment 274, wherein the tissue specific promoter is a hGRK1 promoter.

276. The method of embodiment 274, wherein the tissue specific promoter is a RHO promoter.

277. The method of any one of embodiments 270 to 273, wherein the nucleic acid encoding the Cas9 protein is operably linked to a constitutive promoter.

278. The method of embodiment 277, wherein the constitutive promoter is an EF1 alpha promoter, e.g., an EF1 alpha short (EFS) promoter.

279. The method of any one of embodiments 245 to 278, wherein the cell is a stem cell.

280. The method of any one of embodiments 245 to 278, wherein the cell is an iPS cell.

281. The method of any one of embodiments 245 to 278, wherein the cell is a human retinal cell.

282. The method of any one of embodiments 245 to 278, wherein the cell is a human retinal epithelial cell.

283. The method of any one of embodiments 245 to 278, wherein the cell is a human photoreceptor cell.

284. The method of any one of embodiments 245 to 278, wherein the cell is a human retinal progenitor cell.

285. The method of any one of embodiments 245 to 284, wherein the contacting results in cleavage of the RHO gene encoding a P347 mutation.

286. The method of any one of embodiments 245 to 285, wherein the cell contains a human RHO gene having a P347 mutation and contains a human RHO gene not having a P347 mutation.

287. The method of embodiment 286, wherein as a result of the contacting, indels in the human RHO gene not having a P347 mutation occur with a frequency of less than 20%.

288. The method of embodiment 286, wherein as a result of the contacting, indels in the human RHO gene not having a P347 mutation occur with a frequency of less than 10%.

289. The method of embodiment 286, wherein as a result of the contacting, indels in the human RHO gene not having a P347 mutation occur with a frequency of less than 1%.

290. The method of embodiment 286, wherein as a result of the contacting, indels in the human RHO gene not having a P347 mutation occur with a frequency of less than 0.1%.

291. The method of any one of embodiments 286 to 290, wherein the contacting results in preferential cleavage of the RHO gene having a P347 mutation over the RHO gene not having a P347 mutation.

292. The method of embodiment 291, wherein the preferential cleavage of the RHO gene having a P347 mutation over cleavage of the RHO gene not having a P347 mutation is by a factor of at least 1.3.

293. The method of embodiment 291, wherein the preferential cleavage of the RHO gene having a P347 mutation over cleavage of the RHO gene not having a P347 mutation is by a factor of at least 2.

294. The method of embodiment 291, wherein the preferential cleavage of the RHO gene having a P347 mutation over cleavage of the RHO gene not having a P347 mutation is by a factor of at least 2.5.

295. The method of embodiment 291, wherein the preferential cleavage of the RHO gene having a P347 mutation over cleavage of the RHO gene not having a P347 mutation is by a factor of at least 3.

296. The method of embodiment 291, wherein the preferential cleavage of the RHO gene having a P347 mutation over cleavage of the RHO gene not having a P347 mutation is by a factor of at least 4.

297. The method of embodiment 291, wherein the preferential cleavage of the RHO gene having a P347 mutation over cleavage of the RHO gene not having a P347 mutation is by a factor of at least 5.

298. The method of embodiment 291, wherein the preferential cleavage of the RHO gene having a P347 mutation over cleavage of the RHO gene not having a P347 mutation is by a factor of at least 10.

299. The method of embodiment 291, wherein the preferential cleavage of the RHO gene having a P347 mutation over cleavage of the RHO gene not having a P347 mutation is by a factor of at least 100.

300. The method of any one of embodiments 291 to 297, wherein the preferential cleavage of the RHO gene having a P347 mutation over cleavage of the RHO gene not having a P347 mutation is by a factor of up to 5.

301. The method of any one of embodiments 291 to 297, wherein the preferential cleavage of the RHO gene having a P347 mutation over cleavage of the RHO gene not having a P347 mutation is by a factor of up to 10.

302. The method of any one of embodiments 291 to 298, wherein the preferential cleavage of the RHO gene having a P347 mutation over cleavage of the RHO gene not having a P347 mutation is by a factor of up to 11.

303. The method of any one of embodiments 245 to 302, wherein the contacting results in deletion of one or more nucleotides, insertion of one or more nucleotides, or substitution one or more nucleotides in the RHO gene having a P347 mutation.

304. The method of any one of embodiments 245 to 303, wherein the method reduces expression of rhodopsin comprising the P347 mutation in the cell.

305. The method of any one of embodiments 245 to 304, wherein the cell is a cell from a subject having a RHO gene with a P347 mutation or a progeny of such cell.

306. The method of embodiment 305, wherein the contacting is performed ex vivo.

307. The method of embodiment 306, which further comprises returning the contacted cell or a progeny thereof to the subject.

308. The method of embodiment 305, wherein the contacting is performed in vivo.

309. The method of embodiment 308, wherein the contacting is performed in or near an eye of the subject.

310. The method of embodiment 309, wherein the contacting comprises delivering the gRNA, nucleic acid, plurality of nucleic acids, particle, plurality of particles, system, or pharmaceutical composition to the eye by sub-retinal injection.

311. The method of embodiment 309, wherein the contacting comprises delivering the nucleic acid, plurality of nucleic acids, particle, plurality of particles, system, or pharmaceutical composition to the eye by intravitreal injection.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A guide RNA molecule (gRNA) for editing a human RHO gene having a P347 mutation.

2. The gRNA of claim 1, wherein the P347 mutation is a P347L mutation, a P347S mutation, a P347R mutation, a P347Q mutation, a P347T mutation, or a P347A mutation.

3. The gRNA of claim 2, wherein the P347 mutation is a P347L mutation.

4. The gRNA of any one of claims 1 to 3, wherein upon introduction of the gRNA and a DNA endonuclease capable of associating with the gRNA into a human cell containing a human RHO gene having a P347 mutation, the DNA endonuclease cleaves the RHO gene having a P347 mutation.

5. The gRNA of any one of claims 1 to 4, wherein upon introduction of the gRNA and a DNA endonuclease capable of associating with the gRNA into a human cell containing a human RHO gene having a P347 mutation and containing a human RHO gene not having a P347 mutation or into a population of human cells each containing a human RHO gene having a P347 mutation and containing a human RHO gene not having a P347 mutation, indels in the human RHO gene not having a P347 mutation occur with a frequency of less than 20%.

6. The gRNA of any one of claims 1 to 5, which comprises a spacer that is 15 to 30 nucleotides in length.

7. The gRNA of claim 6, wherein the nucleotide sequence of the spacer comprises 15 or more consecutive nucleotides of a reference sequence or comprises a nucleotide sequence that is at least 85% identical to a reference sequence, wherein the reference sequence is:
(SEQ ID NO: 5)
a) GGUCUUAGGCCAGGGCCACC;
(SEQ ID NO: 6)
b) GCCCUGGCCUAAGACCUGCCU;
(SEQ ID NO: 7)
c) CCUAGGCAGGUCUUAGGCCA;
(SEQ ID NO: 8)
d) gCCUAGGCAGGUCUUAGGCCA;
(SEQ ID NO: 9)
e) GACGAGCCAGGUGGCCCUGGCCU;
(SEQ ID NO: 10)
f) GUCCUAGGCAGGUCUUAGGCCAGG;
(SEQ ID NO: 11)
g) UUAGGCCAGGGCCACCUGGCUC;
or
(SEQ ID NO: 12)
h) GCCAGGUGGCCCUGGCCUAAGA.

8. A guide RNA molecule (gRNA) comprising a spacer whose nucleic acid sequence comprises 15 or more consecutive nucleotides from a reference sequence or comprises a nucleotide sequence that is at least 85% identical to a reference sequence, wherein the reference sequence is:
(SEQ ID NO: 5)
a) GGUCUUAGGCCAGGGCCACC;
(SEQ ID NO: 6)
b) GCCCUGGCCUAAGACCUGCCU;
(SEQ ID NO: 7)
c) CCUAGGCAGGUCUUAGGCCA;
(SEQ ID NO: 8)
d) gCCUAGGCAGGUCUUAGGCCA;
(SEQ ID NO: 9)
e) GACGAGCCAGGUGGCCCUGGCCU;
(SEQ ID NO: 10)
f) GUCCUAGGCAGGUCUUAGGCCAGG;
(SEQ ID NO: 11)
g) UUAGGCCAGGGCCACCUGGCUC;
or
(SEQ ID NO: 12)
h) GCCAGGUGGCCCUGGCCUAAGA.

9. The gRNA molecule of claim 7 or claim 8, wherein the nucleotide sequence of the spacer comprises a sequence that is identical to the reference sequence.

10. The gRNA molecule of any one of claims 7 to 9, wherein the reference sequence is GGUCUUAGGCCAGGGCCACC (SEQ ID NO:5).

11. The gRNA molecule of any one of claims 7 to 9, wherein the reference sequence is GCCCUGGCCUAAGACCUGCCU (SEQ ID NO:6).

12. The gRNA molecule of any one of claims 7 to 9, wherein the reference sequence is CCUAGGCAGGUCUUAGGCCA (SEQ ID NO:7).

13. The gRNA molecule of any one of claims 7 to 9, wherein the reference sequence is gCCUAGGCAGGUCUUAGGCCA (SEQ ID NO:8).

14. The gRNA molecule of any one of claims 7 to 9, wherein the reference sequence is GACGAGCCAGGUGGCCCUGGCCU (SEQ ID NO:9).

15. The gRNA molecule of any one of claims 7 to 9, wherein the reference sequence is GUCCUAGGCAGGUCUUAGGCCAGG (SEQ ID NO:10).

16. The gRNA molecule of any one of claims 7 to 9, wherein the reference sequence is UUAGGCCAGGGCCACCUGGCUC (SEQ ID NO:11).

17. The gRNA molecule of any one of claims 7 to 9, wherein the reference sequence is GCCAGGUGGCCCUGGCCUAAGA (SEQ ID NO:12).

18. The gRNA molecule of any one of claims 1 to 17, which is a Cas9 gRNA.

19. The gRNA molecule of claim 18, which is a Streptococcus pyogenes Cas9 (SpCas9) gRNA.

20. The gRNA molecule of claim 18, which is a Neisseria meningitidis Cas9 gRNA.

21. The gRNA molecule of claim 20, which is a Nme2Cas9 gRNA.

22. The gRNA molecule of any one of claims 1 to 21, which is a single guide RNA (sg RNA).

23. The gRNA molecule of claim 22, which comprises a 3′ sgRNA segment.

24. The gRNA molecule of claim 23, wherein the 3′ sgRNA segment has a nucleotide sequence comprising a sequence set forth in Table 3 or Table 4.

25. The gRNA of claim 24, wherein the 3′ sgRNA segment comprises the nucleotide sequence of 3′ sgRNA sequence 8 as set forth in Table 3

26. The gRNA of claim 24, wherein the 3′ sgRNA segment comprises the nucleotide sequence of 3′ sgRNA sequence 14 as set forth in Table 3.

27. The gRNA of claim 24, wherein the 3′ sgRNA segment comprises the nucleotide sequence of 3′ sgRNA sequence 29 as set forth in Table 3.

28. The gRNA of claim 24, wherein the 3′ sgRNA segment comprises the nucleotide sequence of 3′ sgRNA sequence 31 as set forth in Table 3.

29. The gRNA of claim 24, wherein the 3′ sgRNA segment comprises the nucleotide sequence of 3′ sgRNA sequence 7 as set forth in Table 4.

30. The gRNA of claim 24, wherein the 3′ sgRNA segment comprises the nucleotide sequence of 3′ sgRNA sequence 20 as set forth in Table 4.

31. The gRNA of claim 24, wherein the 3′ sgRNA segment comprises the nucleotide sequence of 3′ sgRNA sequence 21 as set forth in Table 4.

32. The gRNA of claim 24, wherein the 3′ sgRNA segment comprises the nucleotide sequence of 3′ sgRNA sequence 22 as set forth in Table 4.

33. The gRNA of any one of claims 23 to 32, wherein the 3′ sgRNA segment comprises one or more uracils at its 3′ end.

34. The gRNA of claim 33, wherein the 3′ sgRNA segment comprises one to eight uracils at its 3′ end.

35. The gRNA of any one of claims 1 to 34, wherein the gRNA is an unmodified gRNA.

36. A nucleic acid encoding the gRNA of any one of claims 1 to 35.

37. The nucleic acid of claim 36, which further comprises a Pol III promoter sequence operably linked to the nucleotide sequence encoding the gRNA, optionally wherein the promoter is a U6 or H1 promoter.

38. The nucleic acid of claim 36 or claim 37, which further encodes a second gRNA.

39. The nucleic acid of claim 38, wherein the second gRNA is a sgRNA.

40. The nucleic acid of claim 38 or claim 39, wherein the second gRNA comprises a spacer sequence that is partially or fully complementary to a target sequence in intron 4 of a human RHO gene.

41. The nucleic acid of claim 40, wherein the spacer sequence of the second gRNA comprises the spacer sequence of sg-Int39, sg-Int103, or sg-Int153 as set forth in Table 13.

42. The nucleic acid of any one of claims 36 to 41, which further encodes a Cas9 protein.

43. The nucleic acid of claim 42, which further comprises a promoter sequence operably linked to the nucleotide sequence encoding the Cas9 protein, optionally wherein the promoter sequence is (a) a tissue-specific promoter, which is optionally a RHO promoter or a hGRK1 promoter, or (b) a constitutive promoter, which is optionally an EF1 alpha promoter, which is optionally an EF1 alpha short (EFS) promoter.

44. A plurality of nucleic acids comprising (a) the gRNA of any one of claims 1 to 35 or the nucleic acid of any one of claims 36 to 43, and (b) a nucleic acid encoding a Cas9 protein.

45. The plurality of nucleic acids of claim 44, further comprising a nucleic acid encoding a second gRNA, optionally wherein the second gRNA comprises a spacer sequence that is partially or fully complementary to a target sequence in intron 4 of a human RHO gene.

46. A particle comprising the gRNA of any one of claims 1 to 35, the nucleic acid of any one of claims 36 to 43, or the plurality of nucleic acids of claim 44 or claim 45.

47. The particle of claim 46, which further comprises a Cas9 protein or a nucleic acid encoding a Cas9 protein.

48. A particle comprising the nucleic acid of any one of claims 36 to 43, wherein the particle is a viral particle.

49. The particle of claim 48, which is an adeno-associated virus (AAV) particle, optionally wherein the particle is a AAV2 particle, a AAV5 particle, a AAV7m8 particle or a AAV8 particle.

50. The particle of any one of claims 48 to 49, which comprises a nucleic acid encoding a Cas9 protein.

51. The particle of claim 50, wherein the nucleic acid encoding the Cas9 protein further comprises a promoter sequence operably linked to the nucleotide sequence encoding the Cas9 protein, optionally wherein the promoter sequence is (a) a tissue-specific promoter, which is optionally a RHO promoter or a hGRK1 promoter, or (b) a constitutive promoter, which is optionally an EF1 alpha promoter, which is optionally an EF1 alpha short (EFS) promoter

52. A plurality of particles comprising the particle of any one of claims 48 to 49 and a particle comprising a nucleic acid encoding a Cas9 protein.

53. A system comprising a Cas9 protein and a gRNA of any one of claims 1 to 35.

54. The system of claim 53, further comprising a second gRNA, optionally wherein the second gRNA comprises a spacer sequence that is partially or fully complementary to a target sequence in intron 4 of a human RHO gene.

55. A pharmaceutical composition comprising (i) the gRNA of any one of claims 1 to 35, the nucleic acid of any one of claims 36 to 43, the plurality of nucleic acids of any one of claims 44 to 45, the particle of any one of claims 46 to 51, the plurality of particles of claim 52, or the system of claim 53 and (ii) a pharmaceutically acceptable excipient.

56. A cell comprising the gRNA of any one of claims 1 to 35, the nucleic acid of any one of claims 36 to 43, the plurality of nucleic acids of any one of claims 44 to 45, the particle of any one of claims 46 to 51, the plurality of particles of claim 52, or the system of any one of claims 53 to 54.

57. The cell of claim 56, which is a human cell, which is optionally a human retinal cell, a human retinal epithelial cell, a human photoreceptor cell, a human retinal progenitor cell, a stem cell, an iPS cell, a HEK293T cell, or a HEK293T/17 cell, and optionally wherein the cell is an ex vivo cell.

58. The gRNA of any one of claims 1 to 35, the nucleic acid of any one of claims 36 to 43, the plurality of nucleic acids of any one of claims 44 to 45, the particle of any one of claims 46 to 51, the plurality of particles of claim 52, or the system of any one of claims 53 to 54 or the pharmaceutical composition of claim 55 for use in a method of altering a human cell comprising a RHO gene having a P347 mutation.

59. The gRNA for use, nucleic acid for use, plurality of nucleic acids for use, the particle for use, the plurality of particles for use, the system for use, or pharmaceutical composition for use according to claim 58, wherein the P347 mutation is a P347L mutation, a P347S mutation, a P347R mutation, a P347Q mutation, a P347T mutation, or a P347A mutation.

60. The gRNA for use, nucleic acid for use, plurality of nucleic acids for use, the particle for use, the plurality of particles for use, the system for use, or pharmaceutical composition for use according to claim 59, wherein the P347 mutation is a P347L mutation.


SEQUENCE LISTING (326 sequences)

SEQ ID NO: 1 - protein, 1368 aa, Streptococcus pyogenes [sequence omitted]

SEQ ID NO: 2 - protein, 1082 aa, Neisseria meningitidis
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 3 - protein, 348 aa, Homo sapiens [sequence omitted]

SEQ ID NO: 4 - DNA, 5385 bp, Homo sapiens
ggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcatggaggggtctgggagagtcccgggcttggcggtggtggctgagaggccttctcccttctcctgtcctgtcaatgttatccaaagccctcatatattcagtcaacaaacaccattcatggtgatagccgggctgctgtttgtgcagggctggcactgaacactgccttgatcttatttggagcaatatgcgcttgtctaatttcacagcaagaaaactgagctgaggctcaaagaagtcaagcgccctgctggggcgtcacacagggacgggtgcagagttgagttggaagcccgcatctatctcgggccatgtttgcagcaccaagcctctgtttcccttggagcagctgtgctgagtcagacccaggctgggcactgagggagagctgggcaagccagacccctcctctctgggggcccaagctcagggtgggaagtggattttccattctccagtcattgggtcttccctgtgctgggcaatgggctcggtcccctctggcatcctctgcctcccctctcagcccctgtcctcaggtgcccctccagcctccctgccgcgttccaagtctcctggtgttgagaaccgcaagcagccgctctgaagcagttcctttttgctttagaataatgtcttgcatttaacaggaaaacagatggggtgctgcagggataacagatcccacttaacagagaggaaaactgaggcagggagaggggaagagactcatttagggatgtggccaggcagcaacaagagcctaggtctcctggctgtgatccaggaatatctctgctgagatgcaggaggagacgctagaagcagccattgcaaagctgggtgacggggagagcttaccgccagccacaagcgtctctctgccagccttgccctgtctcccccatgtccaggctgctgcctcggtcccattctcagggaatctctggccattgttgggtgtttgttgcattcaataatcacagatcactcagttctggccagaaggtgggtgtgccacttacgggtggttgttctctgcagggtcagtcccagtttacaaatattgtccctttcactgttaggaatgtcccagtttggttgattaactatatggccactctccctatggaacttcatggggtggtgagcaggacagatgtctgaattccatcatttccttcttcttcctctgggcaaaacattgcacattgcttcatggctcctaggagaggcccccacatgtccgggttatttcatttcccgagaagggagagggaggaaggactgccaattctgggtttccaccacctctgcattccttcccaacaaggaactctgccccacattaggatgcattcttctgctaaacacacacacacacacacacacacacaacacacacacacacacacacacacacacacacacaaaactccctaccgggttcccagttcaatcctgaccccctgatctgattcgtgtcccttatgggcccagagcgctaagcaaataacttcccccattccctggaatttctttgcccagctctcctcagcgtgtggtccctctgccccttccccctcctcccagcaccaagctctctccttccccaaggcctcctcaaatccctctcccactcctggttgccttcctagctaccctctccctgtctaggggggagtgcaccctccttaggcagtggggtctgtgctgaccgcctgctgactgccttgcaggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtaatggcactgagcagaagggaagaagctccgggggctctttgtagggtcctccagtcaggactcaaacccagtagtgtctggttccaggcactgaccttgtatgtctcctggcccaaatgcccactcagggtaggggtgtagggcagaagaagaaacagactctaatgttgctacaagggctggtcccatctcctgagccccatgtcaaacagaatccaagacatcccaacccttcaccttggctgtgcccctaatcctcaactaagctaggcgcaaattccaatcctctttggtctagtaccccgggggcagccccctctaaccttgggcctcagcagcaggggaggccacaccttcctagtgcaggtggccatattgtggccccttggaactgggtcccactcagcctctaggcgattgtctcctaatggggctgagatgagacacagtggggacagtggtttggacaataggactggtgactctggtccccagaggcctcatgtccctctgtctccagaaaattcccactctcacttccctttcctcctcagtcttgctagggtccatttcttaccccttgctgaatttgagcccaccccctggactttttccccatcttctccaatctggcctagttctatcctctggaagcagagccgctggacgctctgggtttcctgaggcccgtccactgtcaccaatatcaggaaccattgccacgtcctaatgacgtgcgctggaagcctctagtttccagaagctgcacaaagatcccttagatactctgtgtgtccatctttggcctggaaaatactctcaccctggggctaggaagacctcggtttgtacaaacttcctcaaatgcagagcctgagggctctccccacctcctcaccaaccctctgcgtggcatagccctagcctcagcgggcagtggatgctggggctgggcatgcagggagaggctgggtggtgtcatctggtaacgcagccaccaaacaatgaagcgacactgattccacaaggtgcatctgcatccccatctgatccattccatcctgtcacccagccatgcagacgtttatgatccccttttccagggagggaatgtgaagccccagaaagggccagcgctcggcagccaccttggctgttcccaagtccctcacaggcagggtctccctacctgcctgtcctcaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggtacgggccggggggtgggcggcctcacggctctgagggtccagcccccagcatgcatctgcggctcctgctccctggaggagccatggtctggacccgggtcccgtgtcctgcaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcaggtgcctactgcgggtgggagggccccagtgccccaggccacaggcgctgcctgccaaggacaagctacttcccagggcaggggagggggctccatcagggttactggcagcagtcttgggtcagcagtcccaatggggagtgtgtgagaaatgcagattcctggccccactcagaactgctgaatctcagggtgggcccaggaacctgcatttccagcaagccctccacaggtggctcagatgctcactcaggtgggagaagctccagtcagctagttctggaagcccaatgtcaaagtcagaaggacccaagtcgggaatgggatgggccagtctccataaagctgaataaggagctaaaaagtcttattctgaggggtaaaggggtaaagggttcctcggagaggtacctccgaggggtaaacagttgggtaaacagtctctgaagtcagctctgccattttctagctgtatggccctgggcaagtcaatttccttctctgtgctttggtttcctcatccatagaaaggtagaaagggcaaaacaccaaactcttggattacaagagataatttacagaacacccttggcacacagagggcaccatgaaatgtcacgggtgacacagcccccttgtgctcagtccctggcatctctaggggtgaggagcgtctgcctagcaggttccctccaggaagctggatttgagtggatggggcgctggaatcgtgaggggcagaagcaggcaaagggtcggggcgaacctcactaacgtgccagttccaagcacactgtgggcagccctggccctgactcaagcctcttgccttccagttccggaactgcatgctcaccaccatctgctgcggcaagaacccactgggtgacgatgaggcctctgctaccgtgtccaagacggagacgagccaggtggccccggcctaagacctgcctaggactctgtggccgactataggcgtctcccatcccctacaccttcccccagccacagccatcccaccaggagcagcgcctgtgcagaatgaacgaagtcacataggctccttaattttttttttttttttaagaaataattaatgaggctcctcactcacctgggacagcctgagaagggacatccacca

SEQ ID NO: 5 - RNA, 20 nt, Artificial Sequence
  source: /note=“Description of Artificial Sequence Synthetic oligonucleotide”
ggucuuaggccagggccacc

SEQ ID NO: 6 - RNA, 21 nt, Artificial Sequence
  source: /note=“Description of Artificial Sequence Synthetic oligonucleotide”
gcccuggccuaagaccugccu

SEQ ID NO: 7 - RNA, 20 nt, Artificial Sequence
  source: /note=“Description of Artificial Sequence Synthetic oligonucleotide”
ccuaggcaggucuuaggcca

SEQ ID NO: 8 - RNA, 21 nt, Artificial Sequence
  source: /note=“Description of Artificial Sequence Synthetic oligonucleotide”
gccuaggcaggucuuaggcca

SEQ ID NO: 9 - RNA, 23 nt, Artificial Sequence
  source: /note=“Description of Artificial Sequence Synthetic oligonucleotide”
gacgagccagguggcccuggccu

SEQ ID NO: 10 - RNA, 24 nt, Artificial Sequence
  source: /note=“Description of Artificial Sequence Synthetic oligonucleotide”
guccuaggcaggucuuaggccagg

SEQ ID NO: 11 - RNA, 22 nt, Artificial Sequence
  source: /note=“Description of Artificial Sequence Synthetic oligonucleotide”
uuaggccagggccaccuggcuc

SEQ ID NO: 12 - RNA, 22 nt, Artificial Sequence
  source: /note=“Description of Artificial Sequence Synthetic oligonucleotide”
gccagguggcccuggccuaaga

SEQ ID NO: 13 - DNA, 23 bp, Homo sapiens
tcctcggagaggtacctccgagg

SEQ ID NO: 14 - DNA, 23 bp, Homo sapiens
cctcggagaggtacctccgaggg

SEQ ID NO: 15 - DNA, 23 bp, Homo sapiens
cccaactgtttacccctcggagg

SEQ ID NO: 16 - DNA, 23 bp, Homo sapiens
ctcggagaggtacctccgagggg

SEQ ID NO: 17 - DNA, 23 bp, Homo sapiens
tggggcgctggaatcgtgagggg

SEQ ID NO: 18 - DNA, 23 bp, Homo sapiens
ttggaactggcacgttagtgagg

SEQ ID NO: 19 - DNA, 23 bp, Homo sapiens
gcccatcccattcccgacttggg

SEQ ID NO: 20 - DNA, 23 bp, Homo sapiens
gaaggacccaagtcgggaatggg

SEQ ID NO: 21 - DNA, 23 bp, Homo sapiens
cctccgaggggtaaacagttggg

SEQ ID NO: 22 - DNA, 23 bp, Homo sapiens
ggcccatcccattcccgacttgg

SEQ ID NO: 23 - DNA, 23 bp, Homo sapiens
atggggcgctggaatcgtgaggg

SEQ ID NO: 24 - DNA, 23 bp, Homo sapiens
acctccgaggggtaaacagttgg

SEQ ID NO: 25 - DNA, 23 bp, Homo sapiens
gatggggcgctggaatcgtgagg

SEQ ID NO: 26 - DNA, 23 bp, Homo sapiens
gggtaaagggttcctcggagagg

SEQ ID NO: 27 - DNA, 23 bp, Homo sapiens
ccctcggaggtacctctccgagg

SEQ ID NO: 28 - DNA, 23 bp, Homo sapiens
ggcttccagaactagctgactgg

SEQ ID NO: 29 - DNA, 23 bp, Homo sapiens
agaaggacccaagtcgggaatgg

SEQ ID NO: 30 - DNA, 23 bp, Homo sapiens
cttgggtcagcagtcccaatggg

SEQ ID NO: 31 - DNA, 23 bp, Homo sapiens
gtgtcacccgtgacatttcatgg

SEQ ID NO: 32 - DNA, 23 bp, Homo sapiens
ttgggtcagcagtcccaatgggg

SEQ ID NO: 33 - DNA, 23 bp, Homo sapiens
aagctccagtcagctagttctgg

SEQ ID NO: 34 - DNA, 23 bp, Homo sapiens
ttacccaactgtttacccctcgg

SEQ ID NO: 35 - DNA, 23 bp, Homo sapiens
ggggccctcccacccgcagtagg

SEQ ID NO: 36 - DNA, 23 bp, Homo sapiens
gggggctccatcagggttactgg

SEQ ID NO: 37 - DNA, 23 bp, Homo sapiens
gtgaggagcgtctgcctagcagg

SEQ ID NO: 38 - DNA, 23 bp, Homo sapiens
gatttgagtggatggggcgctgg

SEQ ID NO: 39 - DNA, 23 bp, Homo sapiens
gggttactggcagcagtcttggg

SEQ ID NO: 40 - DNA, 23 bp, Homo sapiens
gacccaagtcgggaatgggatgg

SEQ ID NO: 41 - DNA, 23 bp, Homo sapiens
tcctcacccctagagatgccagg

SEQ ID NO: 42 - DNA, 23 bp, Homo sapiens
gataatttacagaacacccttgg

SEQ ID NO: 43 - DNA, 23 bp, Homo sapiens
acccaagtcgggaatgggatggg

SEQ ID NO: 44 - DNA, 23 bp, Homo sapiens
agtcttattctgaggggtaaagg

SEQ ID NO: 45 - DNA, 23 bp, Homo sapiens
tcttgggtcagcagtcccaatgg

SEQ ID NO: 46 - DNA, 23 bp, Homo sapiens
ggtccttctgactttgacattgg

SEQ ID NO: 47 - DNA, 23 bp, Homo sapiens
ggaagtagcttgtccttggcagg

SEQ ID NO: 48 - DNA, 23 bp, Homo sapiens
agggttactggcagcagtcttgg

SEQ ID NO: 49 - DNA, 23 bp, Homo sapiens
agtctccataaagctgaataagg

SEQ ID NO: 50 - DNA, 23 bp, Homo sapiens
taaaggggtaaagggttcctcgg

SEQ ID NO: 51 - DNA, 23 bp, Homo sapiens
cagaagcaggcaaagggtcgggg

SEQ ID NO: 52 - DNA, 23 bp, Homo sapiens
gagggcaccatgaaatgtcacgg

SEQ ID NO: 53 - DNA, 23 bp, Homo sapiens
cctcacccctagagatgccaggg

SEQ ID NO: 54 - DNA, 23 bp, Homo sapiens
tgccagttccaagcacactgtgg

SEQ ID NO: 55 - DNA, 23 bp, Homo sapiens
actgctgccagtaaccctgatgg

SEQ ID NO: 56 - DNA, 23 bp, Homo sapiens
atttccagcaagccctccacagg

SEQ ID NO: 57 - DNA, 23 bp, Homo sapiens
tagctccttattcagctttatgg

SEQ ID NO: 58 - DNA, 23 bp, Homo sapiens
ctcagtccctggcatctctaggg

SEQ ID NO: 59 - DNA, 23 bp, Homo sapiens
tcagtccctggcatctctagggg

SEQ ID NO: 60 - DNA, 23 bp, Homo sapiens
gaagctggatttgagtggatggg

SEQ ID NO: 61 - DNA, 23 bp, Homo sapiens
tttctcacacactccccattggg

SEQ ID NO: 62 - DNA, 23 bp, Homo sapiens
tccagcaagccctccacaggtgg

SEQ ID NO: 63 - DNA, 23 bp, Homo sapiens
ggtggctcagatgctcactcagg

SEQ ID NO: 64 - DNA, 23 bp, Homo sapiens
caaggacaagctacttcccaggg

SEQ ID NO: 65 - DNA, 23 bp, Homo sapiens
gccacctgtggagggcttgctgg

SEQ ID NO: 66 - DNA, 23 bp, Homo sapiens
gtcttattctgaggggtaaaggg

SEQ ID NO: 67 - DNA, 23 bp, Homo sapiens
acaagctacttcccagggcaggg

SEQ ID NO: 68 - DNA, 23 bp, Homo sapiens
gctcagatgctcactcaggtggg

SEQ ID NO: 69 - DNA, 23 bp, Homo sapiens
aaagtcagaaggacccaagtcgg

SEQ ID NO: 70 - DNA, 23 bp, Homo sapiens
gacaagctacttcccagggcagg

SEQ ID NO: 71 - DNA, 23 bp, Homo sapiens
tctcttgtaatccaagagtttgg

SEQ ID NO: 72 - DNA, 23 bp, Homo sapiens
cctgggaagtagcttgtccttgg

SEQ ID NO: 73 - DNA, 23 bp, Homo sapiens
agcatctgagccacctgtggagg

SEQ ID NO: 74 - DNA, 23 bp, Homo sapiens
gtccttctgactttgacattggg

SEQ ID NO: 75 - DNA, 23 bp, Homo sapiens
gctaaaaagtcttattctgaggg

SEQ ID NO: 76 - DNA, 23 bp, Homo sapiens
tcttattctgaggggtaaagggg

SEQ ID NO: 77 - DNA, 23 bp, Homo sapiens
tccttggcaggcagcgcctgtgg

SEQ ID NO: 78 - DNA, 23 bp, Homo sapiens
atttctcacacactccccattgg

SEQ ID NO: 79 - DNA, 23 bp, Homo sapiens
tggtgccctctgtgtgccaaggg

SEQ ID NO: 80 - DNA, 23 bp, Homo sapiens
agggcaccatgaaatgtcacggg

SEQ ID NO: 81 - DNA, 23 bp, Homo sapiens
agcccaatgtcaaagtcagaagg

SEQ ID NO: 82 - DNA, 23 bp, Homo sapiens
tctgaggggtaaaggggtaaagg

SEQ ID NO: 83 - DNA, 23 bp, Homo sapiens
gaacacccttggcacacagaggg

SEQ ID NO: 84 - DNA, 23 bp, Homo sapiens
gctcagtccctggcatctctagg

SEQ ID NO: 85 - DNA, 23 bp, Homo sapiens
gcaggggagggggctccatcagg

SEQ ID NO: 86 - DNA, 23 bp, Homo sapiens
ccctggcatctctaggggtgagg

SEQ ID NO: 87 - DNA, 23 bp, Homo sapiens
gcccacagtgtgcttggaactgg

SEQ ID NO: 88 - DNA, 23 bp, Homo sapiens
gccagttccaagcacactgtggg

SEQ ID NO: 89 - DNA, 23 bp, Homo sapiens
tcatccatagaaaggtagaaagg

SEQ ID NO: 90 - DNA, 23 bp, Homo sapiens
ctgcctagcaggttccctccagg

SEQ ID NO: 91 - DNA, 23 bp, Homo sapiens
ccaaggacaagctacttcccagg

SEQ ID NO: 92 - DNA, 23 bp, Homo sapiens
caggggagggggctccatcaggg

SEQ ID NO: 93 - DNA, 23 bp, Homo sapiens
gaactgctgaatctcagggtggg

SEQ ID NO: 94 - DNA, 23 bp, Homo sapiens
ggctcagatgctcactcaggtgg

SEQ ID NO: 95 - DNA, 23 bp, Homo sapiens
ttattcagctttatggagactgg

SEQ ID NO: 96 - DNA, 23 bp, Homo sapiens
agggcaaaacaccaaactcttgg

SEQ ID NO: 97 - DNA, 23 bp, Homo sapiens
agaactgctgaatctcagggtgg

SEQ ID NO: 98 - DNA, 23 bp, Homo sapiens
ctgaatctcagggtgggcccagg

SEQ ID NO: 99 - DNA, 23 bp, Homo sapiens
agctaaaaagtcttattctgagg

SEQ ID NO: 100 - DNA, 23 bp, Homo sapiens
tttctacctttctatggatgagg

SEQ ID NO: 101 - DNA, 23 bp, Homo sapiens
tgccagggactgagcacaagggg

SEQ ID NO: 102 - DNA, 23 bp, Homo sapiens
gcaggttccctccaggaagctgg

SEQ ID NO: 103 - DNA, 23 bp, Homo sapiens
aagctggatttgagtggatgggg

SEQ ID NO: 104 - DNA, 23 bp, Homo sapiens
ctcagaactgctgaatctcaggg

SEQ ID NO: 105 - DNA, 23 bp, Homo sapiens
gtgagcatctgagccacctgtgg

SEQ ID NO: 106 - DNA, 23 bp, Homo sapiens
agaacacccttggcacacagagg

SEQ ID NO: 107 - DNA, 23 bp, Homo sapiens
tccaggaagctggatttgagtgg

SEQ ID NO: 108 - DNA, 23 bp, Homo sapiens
gaatcgtgaggggcagaagcagg

SEQ ID NO: 109 - DNA, 23 bp, Homo sapiens
caagctacttcccagggcagggg

SEQ ID NO: 110 - DNA, 23 bp, Homo sapiens
atgccagggactgagcacaaggg

SEQ ID NO: 111 - DNA, 23 bp, Homo sapiens
ctcaaatccagcttcctggaggg

SEQ ID NO: 112 - DNA, 23 bp, Homo sapiens
gcagaagcaggcaaagggtcggg

SEQ ID NO: 113 - DNA, 23 bp, Homo sapiens
atggtgccctctgtgtgccaagg

SEQ ID NO: 114 - DNA, 23 bp, Homo sapiens
ggaagctggatttgagtggatgg

SEQ ID NO: 115 - DNA, 23 bp, Homo sapiens
aagcacactgtgggcagccctgg

SEQ ID NO: 116 - DNA, 23 bp, Homo sapiens
gaaggcaagaggcttgagtcagg

SEQ ID NO: 117 - DNA, 23 bp, Homo sapiens
gccacaggcgctgcctgccaagg

SEQ ID NO: 118 - DNA, 23 bp, Homo sapiens
ggcagaagcaggcaaagggtcgg

SEQ ID NO: 119 - DNA, 23 bp, Homo sapiens
agggctgcccacagtgtgcttgg

SEQ ID NO: 120 - DNA, 23 bp, Homo sapiens
actcaaatccagcttcctggagg

SEQ ID NO: 121 - DNA, 23 bp, Homo sapiens
cgcctgtggcctggggcactggg

SEQ ID NO: 122 - DNA, 23 bp, Homo sapiens
actcagaactgctgaatctcagg

SEQ ID NO: 123 - DNA, 23 bp, Homo sapiens
gcaggcagcgcctgtggcctggg

SEQ ID NO: 124 - DNA, 23 bp, Homo sapiens
cttcctggagggaacctgctagg

SEQ ID NO: 125 - DNA, 23 bp, Homo sapiens
ggcaggcagcgcctgtggcctgg

SEQ ID NO: 126 - DNA, 23 bp, Homo sapiens
tgagattcagcagttctgagtgg

SEQ ID NO: 127 - DNA, 23 bp, Homo sapiens
ctaaaaagtcttattctgagggg

SEQ ID NO: 128 - DNA, 23 bp, Homo sapiens
ttgccctttctacctttctatgg

SEQ ID NO: 129 - DNA, 23 bp, Homo sapiens
gagattcagcagttctgagtggg

SEQ ID NO: 130 - DNA, 23 bp, Homo sapiens
aaggcaagaggcttgagtcaggg

SEQ ID NO: 131 - DNA, 23 bp, Homo sapiens
aagaggcttgagtcagggccagg

SEQ ID NO: 132 - DNA, 23 bp, Homo sapiens
agattcagcagttctgagtgggg

SEQ ID NO: 133 - DNA, 23 bp, Homo sapiens
caggcagcgcctgtggcctgggg

SEQ ID NO: 134 - DNA, 23 bp, Homo sapiens
ggagggcttgctggaaatgcagg

SEQ ID NO: 135 - DNA, 23 bp, Homo sapiens
agaggcttgagtcagggccaggg

SEQ ID NO: 136 - DNA, 23 bp, Homo sapiens
gccccagtgccccaggccacagg

SEQ ID NO: 137 - DNA, 23 bp, Homo sapiens
tgaggggcagaagcaggcaaagg

SEQ ID NO: 138 - DNA, 23 bp, Homo sapiens
gctacttcccagggcaggggagg

SEQ ID NO: 139 - DNA, 23 bp, Homo sapiens
gcatctgagccacctgtggaggg

SEQ ID NO: 140 - DNA, 23 bp, Homo sapiens
gcgcctgtggcctggggcactgg

SEQ ID NO: 141 - DNA, 23 bp, Homo sapiens
ctacttcccagggcaggggaggg

SEQ ID NO: 142 - DNA, 23 bp, Homo sapiens
gccagggactgagcacaaggggg

SEQ ID NO: 143 - DNA, 23 bp, Homo sapiens
tacttcccagggcaggggagggg

SEQ ID NO: 144 - DNA, 23 bp, Homo sapiens
cagcagttctgagtggggccagg

SEQ ID NO: 145 - DNA, 23 bp, Homo sapiens
ctgaggggtaaaggggtaaaggg

SEQ ID NO: 146 - DNA, 23 bp, Homo sapiens
ttgctggaaatgcaggttcctgg

SEQ ID NO: 147 - DNA, 23 bp, Homo sapiens
catccatagaaaggtagaaaggg

SEQ ID NO: 148 - DNA, 23 bp, Homo sapiens
gcctgtggcctggggcactgggg

SEQ ID NO: 149 - DNA, 23 bp, Homo sapiens
gatgccagggactgagcacaagg

SEQ ID NO: 150 - DNA, 23 bp, Homo sapiens
tgggagggccccagtgccccagg

SEQ ID NO: 151 - DNA, 23 bp, Homo sapiens
tgctggaaatgcaggttcctggg

SEQ ID NO: 152 - DNA, 23 bp, Homo sapiens
gaggggcagaagcaggcaaaggg

SEQ ID NO: 153 - DNA, 23 bp, Homo sapiens
gcccccttgtgctcagtccctgg

SEQ ID NO: 154 - DNA, 23 bp, Homo sapiens
acttcccagggcaggggaggggg

SEQ ID NO: 155 - DNA, 23 bp, Homo sapiens
caatttccttctctgtgctttgg

SEQ ID NO: 156 - DNA, 23 bp, Homo sapiens
tggagccccctcccctgccctgg

SEQ ID NO: 157 - DNA, 23 bp, Homo sapiens
aagtcagaaggacccaagtcggg

SEQ ID NO: 158 - DNA, 23 bp, Homo sapiens
attttctagctgtatggccctgg

SEQ ID NO: 159 - DNA, 23 bp, Homo sapiens
ttttctagctgtatggccctggg

SEQ ID NO: 160 - DNA, 23 bp, Homo sapiens
ggagccccctcccctgccctggg

SEQ ID NO: 161 - DNA, 23 bp, Homo sapiens
agggccatacagctagaaaatgg

SEQ ID NO: 162 - DNA, 23 bp, Homo sapiens
tctgccattttctagctgtatgg

SEQ ID NO: 163 - DNA, 23 bp, Homo sapiens
tccactcaaatccagcttcctgg

SEQ ID NO: 164 - DNA, 23 bp, Homo sapiens
agaaggaaattgacttgcccagg

SEQ ID NO: 165 - DNA, 23 bp, Homo sapiens
gtgtgagaaatgcagattcctgg

SEQ ID NO: 166 - DNA, 23 bp, Homo sapiens
tggtttcctcatccatagaaagg

SEQ ID NO: 167 - DNA, 23 bp, Homo sapiens
gaaggaaattgacttgcccaggg

SEQ ID NO: 168 - DNA, 23 bp, Homo sapiens
aggaaaccaaagcacagagaagg

SEQ ID NO: 169 - RNA, 82 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcuuuuuu

SEQ ID NO: 170 - RNA, 81 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcuuuuu

SEQ ID NO: 171 - RNA, 80 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcuuuu

SEQ ID NO: 172 - RNA, 79 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcuuu

SEQ ID NO: 173 - RNA, 78 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcuu

SEQ ID NO: 174 - RNA, 77 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcu

SEQ ID NO: 175 - RNA, 76 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugc

SEQ ID NO: 176 - RNA, 92 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugcuuuuuu

SEQ ID NO: 177 - RNA, 91 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugcuuuuu

SEQ ID NO: 178 - RNA, 90 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugcuuuu

SEQ ID NO: 179 - RNA, 89 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugcuuu

SEQ ID NO: 180 - RNA, 88 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugcuu

SEQ ID NO: 181 - RNA, 87 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugcu

SEQ ID NO: 182 - RNA, 86 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugc

SEQ ID NO: 183 - RNA, 84 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcuuuuuuuu

SEQ ID NO: 184 - RNA, 90 nt, Artificial Sequence
  source: /note=“Description of Artificial Sequence Synthetic oligonucleotide”
guuuaaguacucugugcuggaaacagcacagaaucuacuuaaacaaggcaaaaugccguguuuaucucgucaacuuguuggcgagauuuu

SEQ ID NO: 185 - RNA, 86 nt, Artificial Sequence
  source: /note=“Description of Artificial Sequence Synthetic oligonucleotide”
guuuaaguacucugugcuggaaacagcacagaaucuacuuaaacaaggcaaaaugccguguuuaucucgucaacuuguuggcgaga

SEQ ID NO: 186 - RNA, 94 nt, Artificial Sequence
  source: /note=“Description of Artificial Sequence Synthetic oligonucleotide”
guuuaaguacucugugcuggaaacagcacagaaucuacuuaaacaaggcaaaaugccguguuuaucucgucaacuuguuggcgagauuuuuuuu

SEQ ID NO: 187 - RNA, 92 nt, Artificial Sequence
  source: /note=“Description of Artificial Sequence Synthetic oligonucleotide”
guuuuaguacucuguaaugaaaauuacagaaucuacuaaaacaaggcaaaaugccguguuuaucucgucaacuuguuggcgagauuuuuuuu

SEQ ID NO: 188 - RNA, 84 nt, Artificial Sequence
  source: /note=“Description of Artificial Sequence Synthetic oligonucleotide”
guuuuaguacucuguaaugaaaauuacagaaucuacuaaaacaaggcaaaaugccguguuuaucucgucaacuuguuggcgaga

SEQ ID NO: 189 - RNA, 80 nt, Artificial Sequence
  source: /note=“Description of Artificial Sequence Synthetic oligonucleotide”
guuuuaguacucuggaaacagaaucuacuaaaacaaggcaaaaugccguguuuaucucgucaacuuguuggcgagauuuu

SEQ ID NO: 190 - RNA, 76 nt, Artificial Sequence
  source: /note=“Description of Artificial Sequence Synthetic oligonucleotide”
guuuuaguacucuggaaacagaaucuacuaaaacaaggcaaaaugccguguuuaucucgucaacuuguuggcgaga

SEQ ID NO: 191 - RNA, 84 nt, Artificial Sequence
  source: /note=“Description of Artificial Sequence Synthetic oligonucleotide”
guuuuaguacucuggaaacagaaucuacuaaaacaaggcaaaaugccguguuuaucucgucaacuuguuggcgagauuuuuuuu

SEQ ID NO: 192 - RNA, 93 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugcuuuuuuu

SEQ ID NO: 193 - RNA, 127 nt, Artificial Sequence
  source: /note=“Description of Artificial Sequence Synthetic polynucleotide”
guuguagcucccuuucucauuucggaaacgaaaugagaaccguugcuacaauaaggccgucugaaaagaugugccgcaacgcucugccccuuaaagcuucugcuuuaaggggcaucguuuauuuuuu

SEQ ID NO: 194 - RNA, 126 nt, Artificial Sequence
  source: /note=“Description of Artificial Sequence Synthetic polynucleotide”
guuguagcucccuuucucauuucggaaacgaaaugagaaccguugcuacaauaaggccgucugaaaagaugugccgcaacgcucugccccuuaaagcuucugcuuuaaggggcaucguuuauuuuu

SEQ ID NO: 195 - RNA, 125 nt, Artificial Sequence
  source: /note=“Description of Artificial Sequence Synthetic polynucleotide”
guuguagcucccuuucucauuucggaaacgaaaugagaaccguugcuacaauaaggccgucugaaaagaugugccgcaacgcucugccccuuaaagcuucugcuuuaaggggcaucguuuauuuu

SEQ ID NO: 196 - RNA, 124 nt, Artificial Sequence
  source: /note=“Description of Artificial Sequence Synthetic polynucleotide”
guuguagcucccuuucucauuucggaaacgaaaugagaaccguugcuacaauaaggccgucugaaaagaugugccgcaacgcucugccccuuaaagcuucugcuuuaaggggcaucguuuauuu

SEQ ID NO: 197 - RNA, 123 nt, Artificial Sequence
  source: /note=“Description of Artificial Sequence Synthetic polynucleotide”
guuguagcucccuuucucauuucggaaacgaaaugagaaccguugcuacaauaaggccgucugaaaagaugugccgcaacgcucugccccuuaaagcuucugcuuuaaggggcaucguuuauu

SEQ ID NO: 198 - RNA, 122 nt, Artificial Sequence
  source: /note=“Description of Artificial Sequence Synthetic polynucleotide”
guuguagcucccuuucucauuucggaaacgaaaugagaaccguugcuacaauaaggccgucugaaaagaugugccgcaacgcucugccccuuaaagcuucugcuuuaaggggcaucguuuau

SEQ ID NO: 199 - RNA, 121 nt, Artificial Sequence
  source: /note=“Description of Artificial Sequence Synthetic polynucleotide”
guuguagcucccuuucucauuucggaaacgaaaugagaaccguugcuacaauaaggccgucugaaaagaugugccgcaacgcucugccccuuaaagcuucugcuuuaaggggcaucguuua

SEQ ID NO: 200 - RNA, 118 nt, Artificial Sequence
  source: /note=“Description of Artificial Sequence Synthetic polynucleotide”
guagcucccuuucucauuucggaaacgaaaugagaaccguugcuacaauaaggccgucugaaaagaugugccgcaacgcucugccccuuaaagcuucugcuuuaaggggcaucguuua

SEQ ID NO: 201 - RNA, 94 nt, Artificial Sequence
  source: /note=“Description of Artificial Sequence Synthetic oligonucleotide”
guagcucccgaaacguugcuacaauaaggccgucugaaaagaugugccgcaacgcucugccccuuaaagcuucugcuuuaaggggcaucguuua

SEQ ID NO: 202 - RNA, 84 nt, Artificial Sequence
  source: /note=“Description of Artificial Sequence Synthetic oligonucleotide”
guagcucccgaaacguugcuacaauaaggccgucugaaaagaugugccgcaacgcucugccccuuuucuaaggggcaucguuua

SEQ ID NO: 203 - RNA, 78 nt, Artificial Sequence
  source: /note=“Description of Artificial Sequence Synthetic oligonucleotide”
guagcucccgaaacguugcuacaauaaggccgucugaaaagaugugccgcaacgcucugccccuuuucuaaggggcau

SEQ ID NO: 204 - RNA, 72 nt, Artificial Sequence
  source: /note=“Description of Artificial Sequence Synthetic oligonucleotide”
guagcucccgaaacguugcuacaauaaggccgucugaaaagaugugccgcaacgcucugcuucugcaucguu

SEQ ID NO: 205 - RNA, 74 nt, Artificial Sequence
  source: /note=“Description of Artificial Sequence Synthetic oligonucleotide”
guagcucccgaaacguugcuacaauaaggccgucugaaaagaugugccgcaacgcucugcuucugcaucguuua

SEQ ID NO: 206 - RNA, 76 nt, Artificial Sequence
  source: /note=“Description of Artificial Sequence Synthetic oligonucleotide”
guagcucccgaaacguugcuacaauaaggccgucugaaaagaugugccgcaacgcucugcccuucugggcaucguu

SEQ ID NO: 207 - RNA, 80 nt, Artificial Sequence
  source: /note=“Description of Artificial Sequence Synthetic oligonucleotide”
guagcucccgaaacguugcuacaauaaggccgucugaaaagaugugccgcaacgcucugccccuuucuaggggcaucguu

SEQ ID NO: 208 - RNA, 78 nt, Artificial Sequence
  source: /note=“Description of Artificial Sequence Synthetic oligonucleotide”
guagcucccgaaacguugcuacaauaaggccgucugaaaagaugugccgcaacgcucugccccuucuggggcaucguu

SEQ ID NO: 209 - RNA, 74 nt, Artificial Sequence
  source: /note=“Description of Artificial Sequence Synthetic oligonucleotide”
guagcucccgaaacguugcuacaauaaggccgucugaaaagaugugccgcaacgcucugccuucuggcaucguu

SEQ ID NO: 210 - RNA, 109 nt, Artificial Sequence
  source: /note=“Description of Artificial Sequence Synthetic polynucleotide”
guuguagcucccuuucucgaaagagaaccguugcuacaauaaggccgucugaaaagaugugccgcaacgcucugccccuuaaagcuucugcuuuaaggggcaucguuua

SEQ ID NO: 211 - RNA, 97 nt, Artificial Sequence
  source: /note=“Description of Artificial Sequence Synthetic oligonucleotide”
guuguagcucccgaaacguugcuacaauaaggccgucugaaaagaugugccgcaacgcucugccccuuaaagcuucugcuuuaaggggcaucguuua

SEQ ID NO: 212 - RNA, 77 nt, Artificial Sequence
  source: /note=“Description of Artificial Sequence Synthetic oligonucleotide”
guuguagcucccgaaacguugcuacaauaaggccgucugaaaagaugugccgcaacgcucugccuucuggcaucguu

SEQ ID NO: 213 - protein, 1168 aa, Artificial Sequence
  source: /note=“Description of Artificial Sequence Synthetic polypeptide”
MVPKKKRKVEDKRPAATKKAGQAKKKKMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVREDKRPAATKKAGQAKKKKYPYDVPDYAGYPYDVPDYAGSYPYDVPDYAAAPAAKKKKLD

SEQ ID NO: 214 - RNA, 20 nt, Artificial Sequence
  source: /note=“Description of Artificial Sequence Synthetic oligonucleotide”
ggucuuaggccggggccacc

SEQ ID NO: 215 - RNA, 21 nt, Artificial Sequence
  source: /note=“Description of Artificial Sequence Synthetic oligonucleotide”
gccccggccuaagaccugccu

SEQ ID NO: 216 - RNA, 20 nt, Artificial Sequence
  source: /note=“Description of Artificial Sequence Synthetic oligonucleotide”
ccuaggcaggucuuaggccg

SEQ ID NO: 217 - RNA, 23 nt, Artificial Sequence
  source: /note=“Description of Artificial Sequence Synthetic oligonucleotide”
gacgagccagguggccccggccu

SEQ ID NO: 218 - RNA, 24 nt, Artificial Sequence
  source: /note=“Description of Artificial Sequence Synthetic oligonucleotide”
guccuaggcaggucuuaggccggg

SEQ ID NO: 219 - RNA, 20 nt, Artificial Sequence
  source: /note=“Description of Artificial Sequence Synthetic oligonucleotide”
cuucuccaaugcgacgggug

SEQ ID NO: 220 - DNA, 29 bp, Homo sapiens
gcaggtcttaggccagggccacctggctc

SEQ ID NO: 221 - DNA, 30 bp, Homo sapiens
gtggccctggcctaagacctgcctaggact

SEQ ID NO: 222 - DNA, 29 bp, Homo sapiens
agtcctaggcaggtcttaggccagggcca

SEQ ID NO: 223 - DNA, 29 bp, Homo sapiens
gcaggtcttaggccggggccacctggctc

SEQ ID NO: 224 - DNA, 30 bp, Homo sapiens
gtggccccggcctaagacctgcctaggact

SEQ ID NO: 225 - DNA, 29 bp, Homo sapiens
agtcctaggcaggtcttaggccggggcca

SEQ ID NO: 226 - DNA, 35 bp, Homo sapiens
ggagacgagccaggtggccccggcctaagacctgc

SEQ ID NO: 227 - DNA, 36 bp, Homo sapiens
agagtcctaggcaggtcttaggccggggccacctgg

SEQ ID NO: 228 - DNA, 35 bp, Homo sapiens
ggagacgagccaggtggccctggcctaagacctgc

SEQ ID NO: 229 - DNA, 36 bp, Homo sapiens
agagtcctaggcaggtcttaggccagggccacctgg

SEQ ID NO: 230 - DNA, 34 bp, Homo sapiens
gtcttaggccagggccacctggctcgtctccgtc

SEQ ID NO: 231 - DNA, 34 bp, Homo sapiens
cgagccaggtggccctggcctaagacctgcctag

SEQ ID NO: 232 - DNA, 29 bp, Homo sapiens
gcccttctccaatgcgacgggtgtggtac

SEQ ID NO: 233 - DNA, 24 bp, Artificial Sequence
  source: /note=“Description of Artificial Sequence Synthetic oligonucleotide”
caccggtcttaggccagggccacc

SEQ ID NO: 234 - DNA, 25 bp, Artificial Sequence
  source: /note=“Description of Artificial Sequence Synthetic oligonucleotide”
caccgccctggcctaagacctgcct

SEQ ID NO: 235 - DNA, 25 bp, Artificial Sequence
  source: /note=“Description of Artificial Sequence Synthetic oligonucleotide”
caccgcctaggcaggtcttaggcca

SEQ ID NO: 236 - DNA, 24 bp, Artificial Sequence
  source: /note=“Description of Artificial Sequence Synthetic oligonucleotide”
caccggtcttaggccggggccacc

SEQ ID NO: 237 - DNA, 25 bp, Artificial Sequence
  source: /note=“Description of Artificial Sequence Synthetic oligonucleotide”
caccgccccggcctaagacctgcct

SEQ ID NO: 238 - DNA, 25 bp, Artificial Sequence
  source: /note=“Description of Artificial Sequence Synthetic oligonucleotide”
caccgcctaggcaggtcttaggccg

SEQ ID NO: 239 - DNA, 27 bp, Artificial Sequence
  source: /note=“Description of Artificial Sequence Synthetic oligonucleotide”
caccgacgagccaggtggccccggcct

SEQ ID NO: 240 - DNA, 28 bp, Artificial Sequence
  source: /note=“Description of Artificial Sequence Synthetic oligonucleotide”
caccgtcctaggcaggtcttaggccggg

SEQ ID NO: 241 - DNA, 27 bp, Artificial Sequence
  source: /note=“Description of Artificial Sequence Synthetic oligonucleotide”
caccgacgagccaggtggccctggcct

SEQ ID NO: 242 - DNA, 28 bp, Artificial Sequence
  source: /note=“Description of Artificial Sequence Synthetic oligonucleotide”
caccgtcctaggcaggtcttaggccagg

SEQ ID NO: 243 - DNA, 27 bp, Artificial Sequence
  source: /note=“Description of Artificial Sequence Synthetic oligonucleotide”
caccgttaggccagggccacctggctc

SEQ ID NO: 244 - DNA, 26 bp, Artificial Sequence
  source: /note=“Description of Artificial Sequence Synthetic oligonucleotide”
caacgccaggtggccctggcctaaga

SEQ ID NO: 245 - DNA, 25 bp, Artificial Sequence
  source: /note=“Description of Artificial Sequence Synthetic oligonucleotide”
caccgcttctccaatgcgacgggtg

SEQ ID NO: 246 - DNA, 24 bp, Artificial Sequence
  source: /note=“Description of Artificial Sequence Synthetic oligonucleotide”
aaacggtggccctggcctaagacc

SEQ ID NO: 247 - DNA, 25 bp, Artificial Sequence
  source: /note=“Description of Artificial Sequence Synthetic oligonucleotide”
aaacaggcaggtcttaggccagggc

SEQ ID NO: 248 - DNA, 25 bp, Artificial Sequence
  source: /note=“Description of Artificial Sequence Synthetic oligonucleotide”
aaactggcctaagacctgcctaggc

SEQ ID NO: 249 - DNA, 24 bp, Artificial Sequence
  source: /note=“Description of Artificial Sequence Synthetic oligonucleotide”
aaacggtggccccggcctaagacc

SEQ ID NO: 250 - DNA, 25 bp, Artificial Sequence
  source: /note=“Description of Artificial Sequence Synthetic oligonucleotide”
aaacaggcaggtcttaggccggggc

SEQ ID NO: 251 - DNA, 25 bp, Artificial Sequence
  source: /note=“Description of Artificial Sequence Synthetic oligonucleotide”
aaaccggcctaagacctgcctaggc

SEQ ID NO: 252 - DNA, 27 bp, Artificial Sequence
  source: /note=“Description of Artificial Sequence Synthetic oligonucleotide”
caacaggccggggccacctggctcgtc

SEQ ID NO: 253 - DNA, 28 bp, Artificial Sequence
  source: /note=“Description of Artificial Sequence Synthetic oligonucleotide”
caaccccggcctaagacctgcctaggac

SEQ ID NO: 254 - DNA, 27 bp, Artificial Sequence
  source: /note=“Description of Artificial Sequence Synthetic oligonucleotide”
caacaggccagggccacctggctcgtc

SEQ ID NO: 255 - DNA, 28 bp, Artificial Sequence
  source: /note=“Description of Artificial Sequence Synthetic oligonucleotide”
caaccctggcctaagacctgcctaggac

SEQ ID NO: 256 - DNA, 27 bp, Artificial Sequence
  source: /note=“Description of Artificial Sequence Synthetic oligonucleotide”
caacgagccaggtggccctggcctaac

SEQ ID NO: 257 - DNA, 26 bp, Artificial Sequence
  source: /note=“Description of Artificial Sequence Synthetic oligonucleotide”
caactcttaggccagggccacctggc

SEQ ID NO: 258 - DNA, 25 bp, Artificial Sequence
  source: /note=“Description of Artificial Sequence Synthetic oligonucleotide”
aaaccacccgtcgcattggagaagc

SEQ ID NO: 259 - DNA, 31 bp, Artificial Sequence
  source: /note=“Description of Artificial Sequence Synthetic oligonucleotide”
attaggatccagagtcatccagctggagccc

SEQ ID NO: 260 - DNA, 32 bp, Artificial Sequence
  source: /note=“Description of Artificial Sequence Synthetic oligonucleotide”
taatctcgagtggggtttttcccattcccagg

SEQ ID NO: 261 - DNA, 31 bp, Artificial Sequence
  source: /note=“Description of Artificial Sequence Synthetic oligonucleotide”
cgagccaggtggccctggcctaagacctgcc

SEQ ID NO: 262 - DNA, 31 bp, Artificial Sequence
  source: /note=“Description of Artificial Sequence Synthetic oligonucleotide”
ggcaggtcttaggccagggccacctggctcg

SEQ ID NO: 263 - DNA, 35 bp, Artificial Sequence
  source: /note=“Description of Artificial Sequence Synthetic oligonucleotide”
ttaggatccgccaccatgaatggcacagaaggccc

SEQ ID NO: 264 - DNA, 32 bp, Artificial Sequence
  source: /note=“Description of Artificial Sequence Synthetic oligonucleotide”
taatgaattcttaaggagcctatgtgacttcg

SEQ ID NO: 265 - DNA, 23 bp, Artificial Sequence
  source: /note=“Description of Artificial Sequence Synthetic oligonucleotide”
tccaggaagctggatttgagtgg

SEQ ID NO: 266 - DNA, 22 bp, Artificial Sequence
  source: /note=“Description of Artificial Sequence Synthetic oligonucleotide”
tatgagttgctgctgctgcgag

SEQ ID NO: 267 - DNA, 19 bp, Artificial Sequence
  source: /note=“Description of Artificial Sequence Synthetic oligonucleotide”
accctgggcggtgaaattg

SEQ ID NO: 268 - DNA, 20 bp, Artificial Sequence
  source: /note=“Description of Artificial Sequence Synthetic oligonucleotide”
ggcggaattcttaaggagcc

SEQ ID NO: 269 - DNA, 34 bp, Artificial Sequence
  source: /note=“Description of Artificial Sequence Synthetic oligonucleotide”
taattctagaggagcctcattaattatttcttaa

SEQ ID NO: 270 - DNA, 20 bp, Artificial Sequence
  source: /note=“Description of Artificial Sequence Synthetic oligonucleotide”
cacagtccagtcctgggaac

SEQ ID NO: 271 - DNA, 20 bp, Artificial Sequence
  source: /note=“Description of Artificial Sequence Synthetic oligonucleotide”
tagtagccgggccctacttt

SEQ ID NO: 272 - DNA, 19 bp, Artificial Sequence
  source: /note=“Description of Artificial Sequence Synthetic oligonucleotide”
gtcaccgtccagcacaaga

SEQ ID NO: 273 - DNA, 18 bp, Artificial Sequence
  source: /note=“Description of Artificial Sequence Synthetic oligonucleotide”
ggcaatttcaccgcccag

SEQ ID NO: 274 - DNA, 20 bp, Artificial Sequence
  source: /note=“Description of Artificial Sequence Synthetic oligonucleotide”
acctccgaggggtaaacagt

SEQ ID NO: 275 - DNA, 22 bp, Artificial Sequence
  source: /note=“Description of Artificial Sequence Synthetic oligonucleotide”
ttaaggagcctatgtgacttcg

SEQ ID NO: 276 - DNA, 20 bp, Artificial Sequence
  source: /note=“Description of Artificial Sequence Synthetic oligonucleotide”
aagcgacttgctcccctaac

SEQ ID NO: 277 - DNA, 20 bp, Artificial Sequence
  source: /note=“Description of Artificial Sequence Synthetic oligonucleotide”
gaccacctggtccactatgc

SEQ ID NO: 278 - DNA, 20 bp, Artificial Sequence
  source: /note=“Description of Artificial Sequence Synthetic oligonucleotide”
gtgaggggtgtaacagaaca

SEQ ID NO: 279 - DNA, 20 bp, Artificial Sequence
  source: /note=“Description of Artificial Sequence Synthetic oligonucleotide”
ttgcacctctgactggttgg

SEQ ID NO: 280 - DNA, 20 bp, Artificial Sequence
  source: /note=“Description of Artificial Sequence Synthetic oligonucleotide”
gatggttcaggaagacgggt

SEQ ID NO: 281 - DNA, 20 bp, Artificial Sequence
  source: /note=“Description of Artificial Sequence Synthetic oligonucleotide”
cgggtgatgtgagcaactca

SEQ ID NO: 282 - DNA, 21 bp, Artificial Sequence
  source: /note=“Description of Artificial Sequence Synthetic oligonucleotide”
cagcgagctctagcatttagg

SEQ ID NO: 283 - DNA, 20 bp, Artificial Sequence
  source: /note=“Description of Artificial Sequence Synthetic oligonucleotide”
tcggagtcaacggatttggt

SEQ ID NO: 284 - DNA, 20 bp, Artificial Sequence
  source: /note=“Description of Artificial Sequence Synthetic oligonucleotide”
tcgccccacttgattttgga

SEQ ID NO: 285 - DNA, 20 bp, Artificial Sequence
  source: /note=“Description of Artificial Sequence Synthetic oligonucleotide”
ggtccaggtacatccccgag

SEQ ID NO: 286 - DNA, 20 bp, Artificial Sequence
  source: /note=“Description of Artificial Sequence Synthetic oligonucleotide”
gtgaagacgagctgcccata

SEQ ID NO: 287 - RNA, 20 nt, Artificial Sequence
  source: /note=“Description of Artificial Sequence Synthetic oligonucleotide”
gccaguuccaagcacacugu

SEQ ID NO: 288 - RNA, 20 nt, Artificial Sequence
  source: /note=“Description of Artificial Sequence Synthetic oligonucleotide”
gauggggcgcuggaaucgug

SEQ ID NO: 289 - RNA, 20 nt, Artificial Sequence
  source: /note=“Description of Artificial Sequence Synthetic oligonucleotide”
gugaggagcgucugccuagc

SEQ ID NO: 290 - DNA, 29 bp, Homo sapiens
cgtgccagttccaagcacactgtgggcag

SEQ ID NO: 291 - DNA, 29 bp, Homo sapiens
gtggatggggcgctggaatcgtgaggggc

SEQ ID NO: 292 - DNA, 29 bp, Homo sapiens
ggggtgaggagcgtctgcctagcaggttc

SEQ ID NO: 293 - DNA, 24 bp, Artificial Sequence
  source: /note=“Description of Artificial Sequence Synthetic oligonucleotide”
caccgccagttccaagcacactgt

SEQ ID NO: 294 - DNA, 24 bp, Artificial Sequence
  source: /note=“Description of Artificial Sequence Synthetic oligonucleotide”
caccgatggggcgctggaatcgtg

SEQ ID NO: 295 - DNA, 24 bp, Artificial Sequence
  source: /note=“Description of Artificial Sequence Synthetic oligonucleotide”
caccgtgaggagcgtctgcctagc

SEQ ID NO: 296 - DNA, 24 bp, Artificial Sequence
  source: /note=“Description of Artificial Sequence Synthetic oligonucleotide”
aaacacagtgtgcttggaactggc

SEQ ID NO: 297 - DNA, 24 bp, Artificial Sequence
  source: /note=“Description of Artificial Sequence Synthetic oligonucleotide”
aaaccacgattccagcgccccatc

SEQ ID NO: 298 - DNA, 24 bp, Artificial Sequence
  source: /note=“Description of Artificial Sequence Synthetic oligonucleotide”
aaacgctaggcagacgctcctcac

SEQ ID NO: 299 - RNA, 92 nt, Artificial Sequence
  source: /note=“Description of Artificial Sequence Synthetic oligonucleotide”
guuucagagcuaugcuggaaacagcauagcaaguucaaauaaggcuaguccguuaucaacuugaaaaaguggcaccgagucggugcuuuuuu

SEQ ID NO: 300 - RNA, 92 nt, Artificial Sequence
  source: /note=“Description of Artificial Sequence Synthetic oligonucleotide”
guuugagagcuaugcuggaaacagcauagcaaguucaaauaaggcuaguccguuaucaacuugaaaaaguggcaccgagucggugcuuuuuu

SEQ ID NO: 301 - RNA, 92 nt, Artificial Sequence
  source: /note=“Description of Artificial Sequence Synthetic oligonucleotide”
guuucagagcuacagcagaaaugcuguagcaaguugaaauaaggcuaguccguuaucaacuugaaaaaguggcaccgagucggugcuuuuuu

SEQ ID NO: 302 - DNA, 100 bp, Homo sapiens
cgatgaggcctctgctaccgtgtccaagacggagacgagccaggtggccctggcctaagacctgcctaggactctgtggccgactataggcgtctcccat

SEQ ID NO: 303 - DNA, 23 bp, Artificial Sequence
  source: /note=“Description of Artificial Sequence Synthetic oligonucleotide”
  modified_base (21)..(21): a, c, t, g, unknown or other
ggtcttaggccagggccaccngg

SEQ ID NO: 304 - DNA, 23 bp, Homo sapiens
agtcacaggtcagggccaccagg

SEQ ID NO: 305 - DNA, 23 bp, Homo sapiens
attctcaggtcagggccaccagg

SEQ ID NO: 306 - DNA, 23 bp, Homo sapiens
atgctcaggccagggccaccggc

SEQ ID NO: 307 - DNA, 23 bp, Homo sapiens
tgtctccggccagggccacctgg

SEQ ID NO: 308 - DNA, 23 bp, Homo sapiens
ggtcttaggccagggccacctgg

SEQ ID NO: 309 - DNA, 23 bp, Homo sapiens
agacttagcccaaggccacctgg

SEQ ID NO: 310 - DNA, 23 bp, Homo sapiens
tgtctgaggccagggccgccagg

SEQ ID NO: 311 - DNA, 23 bp, Homo sapiens
agtctgaagtcagggccaccagg

SEQ ID NO: 312 - DNA, 23 bp, Homo sapiens
ggtctgaggtcagggcctcctgg

SEQ ID NO: 313 - DNA, 23 bp, Homo sapiens
aggtctagtccagggccaccagg

SEQ ID NO: 314 - DNA, 23 bp, Homo sapiens
ggtcatgagccagggccaccagg

SEQ ID NO: 315 - DNA, 23 bp, Homo sapiens
cgcattaggccagggacatcagg

SEQ ID NO: 316 - DNA, 23 bp, Homo sapiens
actcttaggtcagggtcacctgg

SEQ ID NO: 317 - DNA, 23 bp, Homo sapiens
cggggtaggccaggaacacctgg

SEQ ID NO: 318 - DNA, 23 bp, Homo sapiens
gatctcaggccagggacacatgg

SEQ ID NO: 319 - DNA, 23 bp, Homo sapiens
aggctgaggccagggccacctag

SEQ ID NO: 320 - DNA, 23 bp, Homo sapiens
atacttagcccaggaccacctgg

SEQ ID NO: 321 - DNA, 23 bp, Homo sapiens
gtaattaggccagggctaccagg

SEQ ID NO: 322 - DNA, 23 bp, Homo sapiens
ggtctcaggccaaggccacaggg

SEQ ID NO: 323 - DNA, 23 bp, Homo sapiens
aagtttaggtaagggccacctgg

SEQ ID NO: 324 - DNA, 23 bp, Homo sapiens
agtcggaggtcaggcccacctgg

SEQ ID NO: 325 - DNA, 23 bp, Homo sapiens
atgcttaggccagggacacttgg

SEQ ID NO: 326 - DNA, 186 bp, Homo sapiens
tctaggggtgaggagcgtctgcctagcaggttccctccaggaagctggatttgagtggatggggcgctggaatcgtgaggggcagaagcaggcaaagggtcggggcgaacctcactaacgtgccagttccaagcacactgtgggcagccctggccctgactcaagcctcttgccttccagttccgg